(19) United States
(12) Patent Application Publication
(10) Pub. No.: US 2024/0384271 A1
(43) Pub. Date: Nov. 21, 2024

(54) TARGETED GENE THERAPY FOR DM-1 MYOTONIC DYSTROPHY

(71) Applicant: Genzyme Corporation, Cambridge, MA (US)

(72) Inventors: Seng Cheng, Cambridge, MA (US); Sarah Melissa Jacobo, Cambridge, MA (US); Takako Moriguchi, Cambridge, MA (US); Catherine O'Riordan, Cambridge, MA (US); Guoxiang Ruan, Cambridge, MA (US)

(21) Appl. No.: 18/626,784

(22) Filed: Apr. 4, 2024

Related U.S. Application Data
(60) Provisional application No. 63/589,417, filed on Oct. 11, 2023.
(60) Provisional application No. 63/494,453, filed on Apr. 5, 2023.

(51) Int. Cl. C12N 15/113; A61K 45/06; A61P 21/00; C12N 15/86

(52) U.S. Cl. CPC C12N 15/113; A61K 45/06; A61P 21/00; C12N 15/86; C12N 2310/14; C12N 2310/531; C12N 2320/33; C12N 2750/14143; C12N 2830/008; C12N 2830/50

(57) ABSTRACT

Provided herein are RNAi molecules for treating myotonic dystrophy type 1 (DM1). Further provided herein are expression cassettes, vectors (e.g., rAAV), viral particles, and pharmaceutical compositions containing the RNAi. Yet further provided herein are methods and kits related to the use of the RNAi, for example, to treat DM1.

### CROSS-REFERENCE TO RELATED APPLICATIONS

This application claims priority to U.S. Provisional Patent Application Ser. No. 63/494,453, filed Apr. 5, 2023; and 63/589,417, filed Oct. 11, 2023; the contents of which are hereby incorporated by references in their entireties.

### SUBMISSION OF SEQUENCE LISTING

The content of the following submission in XML file is incorporated herein by reference in its entirety: a computer readable form (CRF) of the Sequence Listing (file name: 752750_SA9-363B_ST26.xml, date created: Apr. 4, 2024, size: 84,754 bytes).

### FIELD

The present invention relates to variant RNAi molecules. In some aspects, the invention relates to variant RNAi molecules to treat muscular dystrophy.

### BACKGROUND

RNA interference (RNAi) has been shown to be a useful tool for gene silencing in basic research of gene function and shows great promise as a therapeutic agent to suppress genes associated with the development of a number of diseases. In nature, gene regulation by RNAi occurs through small RNAs known as microRNAs (miRNAs) (Ambros, (2004) Nature 431:350-355; Krol et al., (2010) Nat. Rev. Genet. 11:597-610). MicroRNAs have emerged as powerful regulators of diverse cellular processes, and when delivered by viral vectors, artificial miRNAs are continually expressed, resulting in a robust and sustained suppression of target genes. The elucidation of the mechanisms involved in miRNA processing has allowed scientists to co-opt the endogenous cellular RNAi machinery and direct the degradation of a target gene product with the use of artificial miRNAs (see, e.g., US PG Pub. 2014/0163214 and Davidson et al., (2012) Cell 150:873-875).

Myotonic Dystrophy Type-1 (DM1) is a monogenic, autosomal-dominant, progressive disease caused by expansion of CTG repeats (>50) in the DMPK locus. The DMPK with repeats is transcribed into mRNA, which forms hairpins and binds RNA binding proteins, sequestering them from their normal function. This leads to the appearance of nuclear foci, mis-splicing of mRNAs, and ultimately myotonia. DM1 principally affects skeletal, cardiac and smooth muscle, resulting in significant physical, cognitive and behavioral impairments and disability. There is currently no approved therapy for DM1. Therefore, there is a high unmet medical need for therapies to treat DM1.

All references cited herein, including patent applications and publications, are incorporated by reference in their entirety.

### BRIEF SUMMARY

In some aspects, the invention provides a recombinant adeno-associated virus (rAAV) particle comprising an RNAi comprising a first strand and a second strand, wherein the first stand and the second strand for a duplex, the first strand comprises a guide region, wherein the guide region comprises a nucleic acid with the sequence 5′-AGUCGAAGACAGUUCUAGGGU-3′ (SEQ ID NO:1) or with a sequence with about 90% identity to the sequence of SEQ ID NO:1, and the second strand comprises a non-guide region; and an AAV capsid comprising an amino acid sequence with about 90% identity to a wildtype AAVrh74 capsid. In some embodiments, the first strand comprises nucleic acid with the sequence of SEQ ID NO: 1 and the non-guide region comprises nucleic acid with the sequence of SEQ ID NO:2. In some embodiments, the first strand and the second strand are linked by means of a RNA linker capable of forming a loop structure. In some embodiments, the RNA linker comprises from about 4 to about 50 nucleotides. In some embodiments, the loop structure comprises from about 4 to about 20 nucleotides. In some embodiments, the loop structure comprises nucleic sequence with of SEQ ID NO:3 or with a sequence with about 90% identity to the sequence of SEQ ID NO:3. In some embodiments, the RNAi comprises 5′ to 3′ the second strand, the RNA linker, and the first strand. In some embodiments, the RNAi comprises 5′ to 3′ the first strand, the RNA linker, and the second strand. In some embodiments, the RNAi comprises nucleic acid with the sequence of SEQ ID NO: 7 or with a sequence with about 90% identity to the sequence of SEQ ID NO:7. In some embodiments, the RNAi is a small inhibitory RNA (siRNA), a microRNA (miRNA), or a small hairpin RNA (shRNA).

In some embodiments of the invention, the RNAi further comprises a scaffold. In some embodiments, the scaffold comprises all or a portion of the nucleic acid of SEQ ID No: 11. In some embodiments, the miRNA is embedded within the scaffold. In some embodiments, the scaffold has a 5′ arm, wherein the 5′ arm is located 5′ to the nucleic acid encoding the RNAi, and a 3 arm, wherein the 3′ arm is located 3′ to the nucleic acid encoding the RNAi. In some embodiments, the scaffold is a miR-155 scaffold. In some embodiments, the miR-155 scaffold comprises the nucleic acid of SEQ ID NO:9 or a sequence with about 90% identity to the sequence of SEQ ID NO:9 located 5′ to the RNAi. In some embodiments, the miR-155 scaffold comprises the nucleic acid of SEQ ID NO: 10 or a sequence with about 90% identity to the sequence of SEQ ID NO: 10 located 3′ to the RNAi.

In some embodiments of the invention, the RNAi targets RNA encoding a polypeptide associated with myotonic dystrophy-1 (DM1). In some embodiments, the polypeptide is dystrophia myotonica protein kinase (DMPK). In some embodiments, the DMPK comprises a mutation associated with DM1. In some embodiments, the gene encoding DMPK comprises five or more CTG trinucleotide repeats.

In some aspects, the invention provides an expression cassette comprising nucleic acid encoding any of the RNAi described herein. In some embodiments, the nucleic acid encoding the RNAi is operably linked to a promoter. In some embodiments, the promoter is a muscle-specific promotor. In some embodiments, the promoter is a desmin promoter or variant thereof. In some embodiments, the desmin promoter comprises one or more enhancer elements and the promoter for the human desmin gene. In some embodiments, the desmin promoter comprises two enhancer elements and the promoter for the human desmin gene. In some embodiments, the desmin promoter comprises one or more Byme enhancer elements and/or one or more Paulin enhancer elements. In some embodiments, the desmin promoter comprises one or more enhancer elements comprising the nucleotide sequence of SEQ ID NO: 21 or a nucleotide sequence with about 90% identity to the sequence of SEQ ID NO:21 and/or one or more enhancer elements comprising the nucleotide sequence of SEQ ID NO:22 or a nucleotide sequence with about 90% identity to the sequence of SEQ ID NO:22. In some embodiments, the desmin promoter comprises the nucleotide sequence of SEQ ID NO: 12 or a sequence with about 90% identity to the nucleotide sequence of SEQ ID NO: 12. In some embodiments, the expression cassette further comprises an intron. In some embodiments, the intron is a rabbit β-globin intron. In some embodiments, the intron comprises the nucleotide sequence of SEQ ID NO: 13 or a sequence with about 90% identity to the sequence of SEQ ID NO: 13. In some embodiments, the nucleic acid encoding the RNAi is embedded in the intron. In some embodiments, the intron comprises a 5′ arm and a 3′ arm, wherein the 5′ arm is located 5′ to the nucleic acid encoding the RNAi and the 3′ arm is located 3′ to the nucleic acid encoding the RNAi. In some embodiments, the 5′ arm of the intron comprises the nucleotide sequence of SEQ ID NO:14 or a sequence with about 90% identity to the sequence of SEQ ID NO: 14. In some embodiments, the 3′ arm of the intron comprises the nucleotide sequence of SEQ ID NO:15 or a sequence with about 90% identity to the sequence of SEQ ID NO: 15. In some embodiments, the expression cassette further comprises a polyadenylation signal. In some embodiments, the polyadenylation signal is a bovine growth hormone polyadenylation signal, an SV40 polyadenylation signal, or a HSV TK pA. In some embodiments, the polyadenylation signal is a minimal bovine growth hormone polyadenylation signal. In some embodiments, the bovine growth hormone polyadenylation signal comprises the nucleotide sequence of SEQ ID NO: 16 or a sequence with about 90% identity to the sequence of SEQ ID NO: 16. In some embodiments, the expression cassette comprises the nucleotide sequence of SEQ ID NO:17 or a sequence with about 90% identity to the sequence of SEQ ID NO: 17.

In some aspects, the invention provides an expression cassette, wherein the expression cassette comprises a modified desmin promoter, wherein the modified desmin promoter comprises one or more enhancer elements and the promoter for the human desmin gene. In some embodiments, the modified desmin promoter comprises two enhancer elements and the promoter for the human desmin gene. In some embodiments, the modified desmin promoter comprises one or more Byrne enhancer elements and/or one or more Paulin enhancer elements. In some embodiments, the modified desmin promoter comprises one or more enhancer elements comprising the nucleotide sequence of SEQ ID NO:21 or a nucleotide sequence with about 90% identity to the sequence of SEQ ID NO:21 and/or one or more enhancer elements comprising the nucleotide sequence of SEQ ID NO:22 or a nucleotide sequence with about 90% identity to the sequence of SEQ ID NO:22. In some embodiments, the desmin promoter comprises the nucleotide sequence of SEQ ID NO: 12 or a sequence with about 90% identity to the nucleotide sequence of SEQ ID NO: 12. In some embodiments, the expression cassette further comprises an intron. In some embodiments, the intron is a rabbit β-globin intron. In some embodiments, the intron comprises the nucleotide sequence of SEQ ID NO: 13 or a sequence with about 90% identity to the sequence of SEQ ID NO: 13. In some embodiments, the nucleic acid encoding the transgene is embedded in the intron. In some embodiments, the intron comprises a 5′ arm and a 3′ arm, wherein the 5′ arm is located 5′ to the nucleic acid encoding the transgene and the 3′ arm is located 3′ to the nucleic acid encoding the transgene. In some embodiments, the 5′ arm of the intron comprises the nucleotide sequence of SEQ ID NO: 14 or a sequence with about 90% identity to the sequence of SEQ ID NO: 14. In some embodiments, the 3′ arm of the intron comprises the nucleotide sequence of SEQ ID NO: 15 or a sequence with about 90% identity to the sequence of SEQ ID NO: 15. In some embodiments, the expression cassette further comprises a polyadenylation signal. In some embodiments, the polyadenylation signal is a bovine growth hormone polyadenylation signal, an SV40 polyadenylation signal, or a HSV TK pA. In some embodiments, the polyadenylation signal is a minimal bovine growth hormone polyadenylation signal. In some embodiments, the bovine growth hormone polyadenylation signal comprises the nucleotide sequence of SEQ ID NO: 16 or a sequence with about 90% identity to the sequence of SEQ ID NO:16. In some embodiments, the transgene encodes a polypeptide or a nucleic acid. In some embodiments, the transgene encodes an RNAi.

In some aspects, the invention provides a vector comprising any of the expression cassettes described herein. In some embodiments, the expression cassette is flanked by one or more stuffer nucleic acid sequences. In some embodiments, the one or more stuffer nucleic acid sequences is derived from the human SerpinA1 gene. In some embodiments, a stuffer nucleic acid sequence located 5′ to the expression cassette is derived from the human SerpinA1 gene. In some embodiments, a stuffer sequence located 5′ to the expression cassette comprises the nucleotide sequence of SEQ ID NO: 18 or a sequence with about 90% identity to the sequence of SEQ ID NO: 18. In some embodiments, a stuffer nucleic acid sequence located 3′ to the expression cassette is derived from the human SerpinA1 gene. In some embodiments, a stuffer sequence located 3′ to the expression cassette comprises the nucleotide sequence of SEQ ID NO: 19 or a sequence with about 90% identity to the sequence of SEQ ID NO: 19.

In some embodiments of the invention, the vector is a recombinant adeno-associated virus (rAAV) vector. In some embodiments, the expression cassette is flanked by one or more AAV inverted terminal repeat (ITR) sequences. In some embodiments, the expression cassette is flanked by two AAV ITRs. In some embodiments, the AAV ITRs are AAV1, AAV2. AAV3, AAV4, AAV5, AAV6, AAV7, AAV8, AAVrh8, AAVrh8R. AAV9, AAV10, AAVrh10, AAV11, AAV12, AAV2R471A, AAV DJ, a goat AAV, bovine AAV, or mouse AAV serotype ITRs. In some embodiments, the AAV ITRs are AAV2 ITRs. In some embodiments, the rAAV vector comprises the nucleotide sequence of SEQ ID NO:20 or a sequence with about 90% identity to the sequence of SEQ ID NO:20. In some embodiments, the vector is a self-complementary rAAV vector.

In some embodiments, the invention provides a cell comprising any of the expression cassette described herein, any of the vectors described herein, or any of the rAAV vectors described herein.

In some aspects, the invention provides a viral particle comprising any of the vectors described herein. In some aspects, the invention provides a recombinant AAV particle comprising any of the rAAV vectors described herein. In some embodiments, the AAV capsid comprises an amino acid sequence comprising an amino acid substitution at position 502. In some embodiments, the amino acid substitution at position 502 is an isoleucine (I). In some embodiments, the rAAV viral particle comprises a AAVrh74 N502I serotype capsid. In some embodiments, the ITR is an AAV2 ITR and the capsid of the rAAV particle is an AAVrh74 N502I serotype capsid. In some embodiments, the AAV capsid comprises an amino acid sequence comprising an amino acid substitution at position 505. In some embodiments, the amino acid substitution at position 505 is an arginine (R). In some embodiments, the rAAV particle comprises a AAVrh74 W505R serotype capsid. In some embodiments, the ITR is an AAV2 ITR and the capsid of the rAAV particle is an AAVrh74 W505R serotype capsid.

In some aspects, the invention provides a rAAV particle comprising an rAAV vector and a capsid, wherein the rAAV vector comprises the following nucleic acids 5′ to 3′, an AAV2 ITR, nucleic acid encoding a stuffer nucleic acid sequence from the human serpinA1 gene, a Byrne desmin enhancer element, a Paulin desmin enhancer element, a desmin promoter, a 5′ arm of a rabbit β-globin intron, a 5′ miR155 scaffold sequence, a DMPK²⁰⁴ miRNA guide sequence, a miR155 terminal loop sequence, a DMPK²⁰⁴ miRNA passenger sequence, a 3′ miR155 scaffold sequence, a 3′ arm of a rabbit β-globin intron, a minimal bovine growth hormone polyadenylation sequence, nucleic acid encoding a stuffer nucleic acid sequence from the human serpinA1 gene, and an AAV2 ITR; and wherein the capsid is an AAVrh74 N502I capsid.

In some aspects, the invention provides a rAAV particle comprising an rAAV vector, wherein the rAAV vector comprises the following nucleic acids 5′ to 3′, an AAV2 ITR comprising the polynucleotide sequence of SEQ ID NO:43, nucleic acid encoding a stuffer nucleic acid sequence from the human serpinA1 gene comprising the polynucleotide sequence of SEQ ID NO: 18, a Byrne desmin enhancer element comprising the polynucleotide sequence of SEQ ID NO:21, a Paulin desmin enhancer element comprising the polynucleotide sequence of SEQ ID NO:22, a desmin promoter comprising the polynucleotide sequence of SEQ ID NO:23, a 5′ arm of a rabbit β-globin intron comprising the polynucleotide sequence of SEQ ID NO: 14, a 5′ miR155 scaffold sequence comprising the polynucleotide sequence of SEQ ID NO:40, a DMPK²⁰⁴ miRNA guide sequence comprising the polynucleotide sequence of SEQ ID NO:4, a miR 155 terminal loop sequence comprising the polynucleotide sequence of SEQ ID NO:6, a DMPK²⁰⁴ miRNA passenger sequence comprising the polynucleotide sequence of SEQ ID NO:5, a 3′ miR155 scaffold sequence comprising the polynucleotide sequence of SEQ ID NO:41, a 3′ arm of a rabbit β-globin intron comprising the polynucleotide sequence of SEQ ID NO: 15, a minimal bovine growth hormone polyadenylation sequence comprising the polynucleotide sequence of SEQ ID NO: 16, nucleic acid encoding a stuffer nucleic acid sequence from the human serpinA1 gene comprising the polynucleotide sequence of SEQ ID NO: 19, and an AAV2 ITR comprising the polynucleotide sequence of SEQ ID NO:49; and wherein the capsid is an AAVrh74 N502I capsid. In some embodiments, the AAVrh74 N502I capsid comprises capsid proteins comprising the amino acid sequence of SEQ ID NO:50.

In some aspects, the invention provides an rAAV particle comprising an rAAV vector and a capsid, wherein the rAAV vector comprises the following nucleic acids 5′ to 3″, an AAV2 ITR, nucleic acid encoding a stuffer nucleic acid sequence from the human serpinA1 gene, a Byrne desmin enhancer element, a Paulin desmin enhancer element, a desmin promoter, a 5′ arm of a rabbit β-globin intron, a 5′ miR155 scaffold sequence, a DMPK²⁰⁴ miRNA guide sequence, a miR155 terminal loop sequence, a DMPK²⁰⁴ miRNA passenger sequence, a 3′ miR155 scaffold sequence, a 3′ arm of a rabbit β-globin intron, a minimal bovine growth hormone polyadenylation sequence, nucleic acid encoding a stuffer nucleic acid sequence from the human serpinA1 gene, and an AAV2 ITR; and wherein the capsid comprises capsid proteins comprising the amino acid sequence of SEQ ID NO: 50.

In some aspects, the invention provides a rAAV particle comprising an rAAV vector, wherein the rAAV vector comprises the following nucleic acids 5′ to 3′, an AAV2 ITR comprising the polynucleotide sequence of SEQ ID NO:43, nucleic acid encoding a stuffer nucleic acid sequence from the human serpinA1 gene comprising the polynucleotide sequence of SEQ ID NO:18, a Byrne desmin enhancer element comprising the polynucleotide sequence of SEQ ID NO:21, a Paulin desmin enhancer element comprising the polynucleotide sequence of SEQ ID NO:22, a desmin promoter comprising the polynucleotide sequence of SEQ ID NO:23, a 5′ arm of a rabbit β-globin intron comprising the polynucleotide sequence of SEQ ID NO: 14, a 5′ miR155 scaffold sequence comprising the polynucleotide sequence of SEQ ID NO:40, a DMPK²⁰⁴ miRNA guide sequence comprising the polynucleotide sequence of SEQ ID NO:4, a miR 155 terminal loop sequence comprising the polynucleotide sequence of SEQ ID NO:6, a DMPK²⁰⁴ miRNA passenger sequence comprising the polynucleotide sequence of SEQ ID NO:5, a 3′ miR155 scaffold sequence comprising the polynucleotide sequence of SEQ ID NO:41, a 3′ arm of a rabbit β-globin intron comprising the polynucleotide sequence of SEQ ID NO: 15, a minimal bovine growth hormone polyadenylation sequence comprising the polynucleotide sequence of SEQ ID NO: 16, nucleic acid encoding a stuffer nucleic acid sequence from the human serpinA1 gene comprising the polynucleotide sequence of SEQ ID NO: 19, and an AAV2 ITR comprising the polynucleotide sequence of SEQ ID NO:49; and wherein the capsid comprises capsid proteins comprising the amino acid sequence of SEQ ID NO: 50.

In some aspects, the invention provides a rAAV particle comprising an rAAV vector and a capsid, wherein the rAAV vector comprises the following nucleic acids 5′ to 3″, an AAV2 ITR, nucleic acid encoding a stuffer nucleic acid sequence from the human serpinA1 gene, a Byrne desmin enhancer element, a Paulin desmin enhancer element, a desmin promoter, a 5′ arm of a rabbit β-globin intron, a 5′ miR155 scaffold sequence, a DMPK²⁰⁴ miRNA guide sequence, a miR155 terminal loop sequence, a DMPK²⁰⁴ miRNA passenger sequence, a 3′ miR155 scaffold sequence, a 3′ arm of a rabbit β-globin intron, a minimal bovine growth hormone polyadenylation sequence, nucleic acid encoding a stuffer nucleic acid sequence from the human serpinA1 gene, and an AAV2 ITR; and wherein the capsid is an AAVrh74 W505R capsid.

In some embodiments, the invention provides a rAAV particle comprising an rAAV vector, wherein the rAAV vector comprises the following nucleic acids 5′ to 3′, an AAV2 ITR comprising the polynucleotide sequence of SEQ ID NO:43, nucleic acid encoding a stuffer nucleic acid sequence from the human serpinA1 gene comprising the polynucleotide sequence of SEQ ID NO: 18, a Byrne desmin enhancer element comprising the polynucleotide sequence of SEQ ID NO:21, a Paulin desmin enhancer element comprising the polynucleotide sequence of SEQ ID NO:22, a desmin promoter comprising the polynucleotide sequence of SEQ ID NO:23, a 5′ arm of a rabbit β-globin intron comprising the polynucleotide sequence of SEQ ID NO: 14, a 5′ miR155 scaffold sequence comprising the polynucleotide sequence of SEQ ID NO:40, a DMPK²⁰⁴ miRNA guide sequence comprising the polynucleotide sequence of SEQ ID NO:4, a miR 155 terminal loop sequence comprising the polynucleotide sequence of SEQ ID NO:6, a DMPK²⁰⁴ miRNA passenger sequence comprising the polynucleotide sequence of SEQ ID NO:5, a 3′ miR155 scaffold sequence comprising the polynucleotide sequence of SEQ ID NO:41, a 3′ arm of a rabbit β-globin intron comprising the polynucleotide sequence of SEQ ID NO: 15, a minimal bovine growth hormone polyadenylation sequence comprising the polynucleotide sequence of SEQ ID NO: 16, nucleic acid encoding a stuffer nucleic acid sequence from the human serpinA1 gene comprising the polynucleotide sequence of SEQ ID NO: 19, and an AAV2 ITR comprising the polynucleotide sequence of SEQ ID NO:49; and wherein the capsid is an AAVrh74 W505R capsid. In some embodiments, the AAVrh74 W505R capsid comprises capsid proteins comprising the amino acid sequence of SEQ ID NO:52.

In some aspects, the invention provides a rAAV particle comprising an rAAV vector and a capsid, wherein the rAAV vector comprises the following nucleic acids 5′ to 3″, an AAV2 ITR, nucleic acid encoding a stuffer nucleic acid sequence from the human serpinA1 gene, a Byrne desmin enhancer element, a Paulin desmin enhancer element, a desmin promoter, a 5′ arm of a rabbit β-globin intron, a 5′ miR155 scaffold sequence, a DMPK²⁰⁴ miRNA guide sequence, a miR155 terminal loop sequence, a DMPK²⁰⁴ miRNA passenger sequence, a 3′ miR155 scaffold sequence, a 3′ arm of a rabbit β-globin intron, a minimal bovine growth hormone polyadenylation sequence, nucleic acid encoding a stuffer nucleic acid sequence from the human serpinA1 gene, and an AAV2 ITR; and wherein the capsid comprises capsid proteins comprising the amino acid sequence of SEQ ID NO: 52.

In some embodiments, the invention provides a rAAV particle comprising an rAAV vector, wherein the rAAV vector comprises the following nucleic acids 5′ to 3′, an AAV2 ITR comprising the polynucleotide sequence of SEQ ID NO:43, nucleic acid encoding a stuffer nucleic acid sequence from the human serpinA1 gene comprising the polynucleotide sequence of SEQ ID NO: 18, a Byrne desmin enhancer element comprising the polynucleotide sequence of SEQ ID NO:21, a Paulin desmin enhancer element comprising the polynucleotide sequence of SEQ ID NO:22, a desmin promoter comprising the polynucleotide sequence of SEQ ID NO:23, a 5′ arm of a rabbit β-globin intron comprising the polynucleotide sequence of SEQ ID NO: 14, a 5′ miR155 scaffold sequence comprising the polynucleotide sequence of SEQ ID NO:40, a DMPK²⁰⁴ miRNA guide sequence comprising the polynucleotide sequence of SEQ ID NO:4, a miR 155 terminal loop sequence comprising the polynucleotide sequence of SEQ ID NO:6, a DMPK²⁰⁴ miRNA passenger sequence comprising the polynucleotide sequence of SEQ ID NO:5, a 3′ miR 155 scaffold sequence comprising the polynucleotide sequence of SEQ ID NO:41, a 3′ arm of a rabbit β-globin intron comprising the polynucleotide sequence of SEQ ID NO: 15, a minimal bovine growth hormone polyadenylation sequence comprising the polynucleotide sequence of SEQ ID NO: 16, nucleic acid encoding a stuffer nucleic acid sequence from the human serpinA1 gene comprising the polynucleotide sequence of SEQ ID NO: 19, and an AAV2 ITR comprising the polynucleotide sequence of SEQ ID NO:49; and wherein the capsid comprises capsid proteins comprising the amino acid sequence of SEQ ID NO: 52.

In some aspects, the invention provides a composition comprising any of the viral particles or rAAV particles described herein. In some embodiments, the invention provides a pharmaceutical composition comprising any of the viral particles or rAAV particles described herein. In some embodiments, the composition further comprises a pharmaceutically acceptable carrier.

In some aspects, the invention provides kits comprising one or more of an RNAi as described herein, a viral particle as described herein, an AAV particle as described herein, or a composition as described herein. In some embodiments, the kit further comprises instructions for use.

In some aspects, the invention provides methods for treating myotonic dystrophy-1 (DM1) in a mammal in need thereof comprising administering to the mammal an effective amount of any of the RNAi described herein. In some aspects, the invention provides methods for inhibiting the expression of dystrophia myotonica protein kinase (DMPK) in a mammal with DM-1 in need thereof comprising administering to the mammal an effective amount of any of the RNAi described herein. In some aspects, the invention provides methods for inhibiting the accumulation of DMPK RNA in a cell of a mammal with DM-1 in need thereof comprising administering to the mammal an effective amount of any of the RNAi described herein.

In some aspects, the invention provides methods for treating myotonic dystrophy-1 (DM1) in a mammal in need thereof comprising administering to the mammal an effective amount of any of the viral particles (e.g., rAAV particles) as described herein. In some aspects, the invention provides methods for inhibiting the expression of dystrophia myotonica protein kinase (DMPK) in a mammal with DM-1 in need thereof comprising administering to the mammal an effective amount of any of the viral particles (e.g., rAAV particles) as described herein. In some aspects, the invention provides methods for inhibiting the accumulation of DMPK RNA in a cell of a mammal with DM-1 in need thereof comprising administering to the mammal an effective amount of any of the viral particles (e.g., rAAV particles) as described herein.

In some embodiments of the invention, the effective amount of the viral particles (e.g., rAAV particles) is a dose of about 1×10⁸ to about 2×10¹³ genome copies/mL. In some embodiments of the invention, the dose is about 5×10¹² genome copies/mL. In some embodiments of the invention, the dose is about 1×10¹³ genome copies/mL. In some embodiments of the invention, the dose is about 2×10¹³ genome copies/mL.

In some embodiments of the invention, the effective amount of the viral particles (e.g., rAAV particles) is a dose of about 1×10⁸ to about 2×10¹⁴ genome copies/kg of body weight. In some embodiments of the invention, the dose is about 5×10¹³ genome copies/kg of body weight. In some embodiments of the invention, the dose is about 1×10¹⁴ genome copies/kg of body weight. In some embodiments of the invention, the dose is about 2×10¹⁴ genome copies/kg of body weight.

In some aspects, the invention provides methods for treating myotonic dystrophy-1 (DM1) in a mammal in need thereof comprising administering to the mammal an effective amount of any of the compositions as described herein. In some aspects, the invention provides methods for inhibiting the expression of dystrophia myotonica protein kinase (DMPK) in a mammal with DM-1 in need thereof comprising administering to the mammal an effective amount of a the composition of any of the composition as described herein. In some aspects, the invention provides methods for inhibiting the accumulation of DMPK RNA in a cell of a mammal with DM-1 in need thereof comprising administering to the mammal an effective amount of any of the composition as described herein.

In some embodiments of the invention, the RNAi is administered in combination with an immunosuppressive agent, wherein the immunosuppressive agent is administered before, at the same time, and/or after administration of the RNAi. In some embodiments, the viral particle or the rAAV particle is administered in combination with an immunosuppressive agent, wherein the immunosuppressive agent is administered before, at the same time, and/or after administration of the viral particle or the rAAV particle. In some embodiments, the composition is administered in combination with an immunosuppressive agent, wherein the immunosuppressive agent is administered before, at the same time, and/or after administration of the composition.

### DESCRIPTION OF THE DRAWINGS

The present application can be understood by reference to the following description taken in conjunction with the accompanying figures.

FIG. 1A depicts a sequence schematic of nDes-miR155-amiR-DMPK²⁰⁴ gene cassette. A hybrid muscle promoter is located upstream of the miR155-amiR-DMPK²⁰⁴ sequence. Downstream from the miRNA is a bovine growth hormone polyadenylation sequence (minBGHpA). A stuffer sequence from the A1AT intron flanks either side of the cassette. All of these are flanked by two AAV2 ITRs generating a combined vector genome size of 3739 bp.

FIG. 1B depicts the ITR plasmid used for cloning of the nDes-miR155-amiR-DMPK²⁰⁴ gene cassette. The ITR plasmid contains A1AT stuffer sequence flanked by the AAV2 5′ and 3′ ITRs. The A1AT stuffer sequence comprises NcoI and SphI restriction sites for cloning.

FIG. 1C depicts the results of a small-scale packaging assay was performed in HEK 293 cells to confirm packaging of the DC969-nDes-miR155-amiR-DMPK²⁰⁴ plasmid. Small-scale production was performed using the AAV rep/cap plasmid. The y-axis shows the amount of vector produced per HEK 293 cell as compared to a standard EGFP plasmid gene cassette (CD627-CBA-GFP). DRP: DNase resistance particle.

FIGS. 2A-2C depict an evaluation of DMPK knockdown by AAV the nDes-miR155-amiR-DMPK²⁰⁴ (amiR155-204) expression cassette in DMSXL mouse after tail vein injection. FIG. 2A shows transduction efficiency and biodistribution of AAV as evaluated by quantifying transgene copy numbers in the different organs. The qPCR results are expressed as mean ratio of AAV copy number/nuclei. FIG. 2B shows levels of amiR-DMPK²⁰⁴ in transduced tissues. MicroRNA input levels were normalized to U6 small nuclear RNA and set relative to BSS (Balance Salt Solution)-treated cells. FIG. 2C shows silencing of DMPK in transduced tissues. Total DMPK was determined by qRT-PCR. mRNA input was normalized to tata-box-binding protein (TBP) and set relative to BSS-treated cells. The dotted line indicates 50% DMPK expression relative to TBP expression. For FIGS. 2A-2C, data were evaluated using Student's T test, paired: *p<0.05, **p<0.01, ***p<0.001, and ****p<0.0001, n=13 (BSS), n=12 (amiR155-204).

FIGS. 3A-3B depict suppression of human DMPK by AAV nDes-miR155-amiR-DMPK²⁰⁴. DMSXL mice were injected systemically with AAV nDes-miR155-amiR-DMPK²⁰⁴ in a dose dependent manner. The mice were euthanized after 8 weeks, organs were harvested, and amiR-DMPK²⁰⁴ and DMPK transcript levels were measured. FIG. 3A depicts the abundance of amiR-DMPK²⁰⁴ normalized to U6 in various tissues. FIG. 3B depicts the abundance of hDMPK transcripts normalized to mTBP in various tissues. Data were evaluated using ANOVA, multiple-comparison test: *p<0.05, **p<0.01, ***p<0.001, and ****p<0.0001. (n=wt-10, BSS-7, low dose 5, medium dose 13 and high dose 7).

FIG. 4 depicts correction of splicing abnormalities in DMSXL mice after systemic treatment with AAV nDes-miR155-amiR-DMPK²⁰⁴ Splicing of alternative exon 11 in LDB3 was assessed using RT-PCR in gastrocnemius muscle after 8 weeks of treatment. Data were evaluated using ANOVA, multiple-comparison test: *p<0.05, **p<0.01, ***p<0.001, and ****p<0.0001. (n=wt-10, BSS-7, low dose 5, medium dose 13 and high dose 7).

FIGS. 5A and 5B show increased survival rate and body weight in female DMSXL mice treated with AAV nDes-miR155-amiR-DMPK²⁰⁴ in a doses dependent manner. FIG. 5A shows Kaplan-Meier survival curves showing improved survival rate with medium dose after 8 weeks of treatment as compared to low dose or BSS treated animals. FIG. 5B shows improved body weight observed in DMSXL animals treated with AAV nDes-miR155-amiR-DMPK²⁰⁴ as compared to BSS treated or low dose treated animals.

FIG. 6 depicts reversal of electrophysiological features of DM1 disease upon systemic treatment of DMSXL DM1 mouse model with AAV nDes-miR155-amiR-DMPK²⁰⁴ in a dose dependent manner. Data were evaluated using ANOVA, multiple-comparison test: *p<0.05, **p<0.01, ***p<0.001, and ****p<0.0001. (n=wt-10, BSS-7, low dose 5, medium dose 13 and high dose 7).

FIG. 7A shows a schematic representation of experimental protocol. Blood (+) was collected on day-1 to reconfirm the neutralizing antibodies. NHPs were administered with AAV encoding GFP reporter at a dose of 1e13vg/kg on day 0), and necropsy was performed on D21 and collected multiple tissues to evaluate the biodistribution. FIGS. 7B-7E show quantification of GFP expression from various tissues of animals injected with AAV9, AAVrh74 and AAVrh74 N502I (rh74M). Bar graphs represents GFP quantities measured by ELISA in Tibialis anterior muscle (TA: FIG. 7B), Biceps Femoris (FIG. 7C), Quadriceps (FIG. 7D), Heart (FIG. 7E) and Liver (FIG. 7F). Data were evaluated using ANOVA, multiple-comparison test: *p<0.05, **p<0.01, ***p<0.001, and ****p<0.0001.

FIGS. 8A and 8B show suppression of human DMPK by AAV rh74 N502I nDes-miR155-amiR-DMPK²⁰⁴. DMSXL mice were injected systemically with AAV rh74 N502I nDes-miR155-amiR-DMPK²⁰⁴ in a dose dependent manner. The mice were euthanized after 8 weeks, organs were harvested, and amiR-DMPK²⁰⁴ and DMPK transcript levels were measured. FIG. 8A depicts the abundance of amiR-DMPK²⁰⁴ normalized to U6 in various tissues. FIG. 8B depicts the abundance of hDMPK transcripts normalized to mTBP in various tissues. Data were evaluated using ANOVA, multiple-comparison test: *p<0.05, **p<0.01, ***p<0.001, and ****p<0.0001.

FIG. 9 shows silencing of endogenous DMPK by AAVrh74N502I nDes-miR155-amiR-DMPK²⁰⁴. Total DMPK levels were determined by qRT-PCR on RNA extracted from human DM1 cardiomyocytes differentiated from iPSCs. These cardiomyocytes were transduced with AAVrh74N502I nDes-miR155-amiR-DMPK²⁰⁴. mRNA input levels were normalized to TBP mRNA. miRCTL3 served as a negative control and was set at 1. Student's T test, paired: **p<0.01.

FIG. 10 is a Volcano plot showing genome-wide gene expression changes in amiR-DMPK²⁰⁴ treated HEK293 cells. (Benjamini Hochberg FDR <1%). Table showing top four differentially expressed (DE) genes with a 3′ UTR seed complementary with FDR <1% and FDR <5%.

FIG. 11 shows conservation of amiRDMPK²⁰⁴ target sequence among multiple sequences. Human target is SEQ ID NO:28, macaque target is SEQ ID NO:29, mouse target is SEQ ID NO:30, rat target is SEQ ID NO:31, and dog target is SEQ ID NO:32.

FIG. 12 illustrates the biodistribution of viral genome copies/cell in multiple tissues in the indicated treatment groups.

FIG. 13 shows the dose-dependent amiR-DMPK expression in various muscle tissues in the indicated treatment groups.

FIG. 14 shows DMPK expression levels after treatment in various muscles for each indicated treatment group.

FIG. 15A-15F illustrates changes in splicing of 6 genes (MBNL1, SOS1, PKM, zTTN, GOLGA4, and CLASP1), comparing healthy myotubes, untreated DM1 myotubes, and DM1 myotubes treated with amiR-DMPK²⁰⁴.

FIG. 16A-16B illustrates an in vitro screen to identify promoters with high activity in skeletal muscles (human myotubes: FIG. 16A) and low activity in liver cells (Huh7 cells: FIG. 16B). Promoters S1-S9 were compared to published muscle promoters as well as to the ubiquitous CAG promoter.

FIG. 17A-17F illustrates data from immortalized human DM1 myotubes transduced with AAV2 carrying a DMPK-targeting miRNA expressed under the desmin promoter. PCR was used to quantify miRNA (FIG. 17A) and DMPK mRNA levels (FIG. 17B). Nuclear CTG RNA foci were imaged using fluorescent in situ hybridization (FISH) and were quantified in untreated (FIG. 17C) and treated (FIG. 17D) cells. Quantification of foci can be seen in FIG. 17E-17F.

FIG. 18 illustrates DMPK levels in DMSXL mice dosed with 9×10¹³ vg/kg AAV.amiR155-DMPK²⁰⁴. Eight weeks after dosing. DMPK expression was measured by PCR. There was a significant reduction of DMPK in treated versus untreated mice in several muscles, while no significant reduction of DMPK mRNA was detected in the liver.

FIG. 19 illustrates a significant reduction in RNA foci size in mouse hearts following treatment (DMSXL+) compared to a control (buffer: DMSXL−).

FIG. 20 illustrates the splicing of various genes (Ldb3, Mbnl2, Spag9, DNAse1, Tnnt3, and Zbtb49) in wild-type mice, DMSXL mice treated with buffer (DMSXL−), and DMSXL mice that received treatment (DMSXL+).

FIG. 21 depicts the functional benefits in DMSXL mice treated with AAV.amiR155-DMPK²⁰⁴. Myotonia was measured in three different muscle groups-gastrocnemius, quadriceps, and tibialis anterior (TA) in wild-type mice, DMSXL mice that did not receive treatment (DMSXL−) and DMSXL mice that received treatment (DMSXL+) using electromyography (EMG). The number of grade 0 events (no myotonia) vs grade 1-3 events were compared across the groups.

FIG. 22A-22B depict cardiac changes in DMSXL mice with or without AAV.amiR155-DMPK²⁰⁴ treatment compared to WT mice. FIG. 22A is a chart depicting changes in aortic diameter (mm). FIG. 22B depicts changes in cardiac output (μL/s).

### DETAILED DESCRIPTION

In some aspects, the invention provides an RNAi comprising a first strand and a second strand, wherein a) the first strand and the second strand form a duplex: b) the first strand comprises a guide region, wherein the guide region comprises nucleic acid with the sequence 5′-AGUCGAAGACAGUUCUAGGGU-3′ (SEQ ID NO:1) or with a sequence with about 90% identity to the sequence of SEQ ID NO: 1; and c) the second strand comprises a non-guide region, wherein the non-guide region comprises nucleic acid with the sequence 5′-ACCCUAGAUGUCUUCGAUU-3′ (SEQ ID NO:2) or a with a sequence with about 90% identity to the sequence of SEQ ID NO:2. In some embodiments, the invention provides expression cassettes for expressing nucleic acid encoding the RNAi; for example, for expressing the RNAi in muscles of a mammal. In some embodiments, the expression cassette is in an rAAV vector.

In some aspects, the invention provides methods for treating myotonic dystrophy 1 (DM-1) in a mammal by administering the RNAi of the invention to the mammal. In some embodiments, administered RNAi inhibits the expression of dystrophia myotonica protein kinase (DMPK) in the mammal: thereby ameliorating the DM-1 in the mammal.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003): the series Methods in Enzymology (Academic Press, Inc.); PCR 2: A Practical Approach (M. J. MacPherson, B. D. Hames and G. R. Taylor eds., 1995); Antibodies. A Laboratory Manual (Harlow and Lane, eds., 1988); Culture of Animal Cells: A Manual of Basic Technique and Specialized Applications (R. I. Freshney, 6th ed., J. Wiley and Sons, 2010): Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### Definitions

A “vector,” as used herein, refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one, and in some embodiments two, inverted terminal repeat sequences (ITRs).

A “recombinant AAV vector (rAAV vector)” refers to a polynucleotide vector comprising one or more heterologous sequences (i.e., nucleic acid sequence not of AAV origin) that are flanked by at least one, and in some embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

The terms “genome particles (gp),” “genome equivalents,” or “genome copies (gc)” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039: Veldwijk et al. (2002) Mol. Ther., 6:272-278.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting the expression of a gene may refer to any act leading to a blockade, reduction, elimination, or any other antagonism of expression of the gene, including reduction of mRNA abundance (e.g., silencing mRNA transcription), degradation of mRNA, inhibition of mRNA translation, and so forth. In some embodiments, inhibiting the expression of DMPK may refer a blockade, reduction, elimination, or any other antagonism of expression of DMPK, including reduction of DMPK mRNA abundance (e.g., silencing DMPK mRNA transcription), degradation of DMPK mRNA, inhibition of DMPK mRNA translation, and so forth. As another example, inhibiting the accumulation of a protein in a cell may refer to any act leading to a blockade, reduction, elimination, or other antagonism of expression of the protein, including reduction of mRNA abundance (e.g., silencing mRNA transcription), degradation of mRNA, inhibition of mRNA translation, degradation of the protein, and so forth. In some embodiments, inhibiting the accumulation of DMPK protein in a cell refers to a blockade, reduction, elimination, or other antagonism of expression of the DMPK protein in a cell, including reduction of DMPK mRNA abundance (e.g., silencing DMPK mRNA transcription), degradation of DMPK mRNA, inhibition of DMPK mRNA translation, degradation of the DMPK protein, and so forth

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in Mclaughlin et al. (1988) J. Virol., 62:1963-1973.

The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532.

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. A helper virus provides “helper functions” which allow for the replication of AAV. A number of such helper viruses have been identified, including adenoviruses, herpesviruses and, poxviruses such as vaccinia and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions. Baculoviruses available from depositories include Autographa californica nuclear polyhedrosis virus.

A preparation of rAAV is said to be “substantially free” of helper virus if the ratio of infectious AAV particles to infectious helper virus particles is at least about 102:1; at least about 104:1, at least about 106:1; or at least about 108:1 or more. In some embodiments, preparations are also free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

“Percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987). Supp. 30, section 7.7.18. Table 7.7.1, and including BLAST. BLAST-2. ALIGN or Megalign (DNASTAR) software. A preferred alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B. and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D. and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

An “isolated” molecule (e.g., nucleic acid or protein) or cell means it has been identified and separated and/or recovered from a component of its natural environment.

An “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” is an approach for obtaining beneficial or desired clinical results. For purposes of this invention, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

As used herein, the term “myotonic dystrophy type 1” or “DM1” refers to the a multisystem disorder that affects skeletal and smooth muscle as well as the eye, heart, endocrine system, and central nervous system. There are three overlapping categories of DM-1 (Bird, T D, Myotonic Dystrophy Type 1. 1999 Sep. 17 [Updated 2021 Mar. 25]. In: Adam M P, Ardinger H H, Pagon R A, et al., editors. GeneReviews® [Internet]. Seattle (WA): University of Washington, Seattle: 1993-2022). Mild DM1 is characterized by cataract and mild myotonia, Classic DM1 is characterized by muscle weakness and wasting, myotonia, cataract, and often cardiac conduction abnormalities Congenital DM1 is characterized by hypotonia and severe generalized weakness at birth, often with respiratory insufficiency and early death: intellectual disability is common.

As used herein, the term “dystrophia myotonica protein kinase”, “DMPK” “myotonin-protein kinase”, “MT-PK”, “myotonic dystrophy protein kinase” or “MDPK” may refer either to the gene or to a polypeptide product thereof associated with most cases of DM1. The 3′ untranslated region of the DMPK gene contains 5-37 copies of a CTG trinucleotide repeat. Expansion of this unstable motif to 50-1,000 copies causes myotonic dystrophy type I, which increases in severity with increasing repeat element copy number.

As used herein, an “RNAi” may refer to any RNA molecule that induces RNA interference in a cell. Examples of RNAi include without limitation small inhibitory RNAs (siRNAs), microRNAs (miRNAs), and small hairpin RNAs (shRNAs).

An “miRNA” may refer to a polynucleotide containing (i) a double-stranded sequence targeting a gene of interest for knockdown by RNAi and (ii) additional sequences that form a stem-loop structure resembling that of endogenous miRNAs. In some embodiments, the miRNA includes nucleic acid flanking the stem-loop structure. These flanking sequences are knows as the “miRNA scaffold.” A sequence targeting a gene of interest for RNAi (e.g., a short, ˜20-nt sequence) may be ligated to sequences that create a miRNA-like stem-loop and a sequence that base pairs with the sequence of interest to form a duplex when the polynucleotide is assembled into the miRNA-like secondary structure. As described herein, this duplex may hybridize imperfectly, e.g., it may contain one or more unpaired or mispaired bases. Upon cleavage of this polynucleotide by Dicer, this duplex containing the sequence targeting a gene of interest may be unwound and incorporated into the RISC complex. A miRNA scaffold may refer to the miRNA itself or to a DNA polynucleotide encoding the miRNA. Examples of a miRNA scaffold include the miR-155 sequence (Lagos-Quintana, M. et al. (2002) Curr. Biol. 12:735-9) and the mirGE scaffold (WO2014016817A2). Commercially available kits for cloning a sequence into a miRNA scaffold are known in the art (e.g., the Invitrogen™ BLOCK-iT™ Pol II miR RNAi expression vector kit from Life Technologies, Thermo Fisher Scientific: Waltham, MA).

As used herein, the term “DMPK-mediated splicing defects” refers to a dysregulation of alternative splicing that occurs in DM1-affected tissues. These splicing defects contribute to core symptoms of the disease, such as insulin resistance, myotonia, muscle weakness, and cardiac arrhythmia. CUG repeat expansion in DMPK transcripts accumulate in the cell nucleus, impairing the physiological function of proteins implicated in transcription, splicing, or RNA export. These aggregations lead to the deregulation of the alternative splicing of different transcripts due to the alteration of the splicing machinery. In some embodiments, gene transcripts known to have dysregulated splicing in DM1 may be used to measure the effect of treatment with the constructs as described herein. In some embodiments, rescue of splicing may be measured. In some embodiments, rescue of splicing may be about 5%, about 10%, about 20%, about 30%, about 40%, about 50%, about 60%, about 70%, about 80%, about 90%, about 100% compared to comparable healthy tissue. In some embodiments, the gene transcripts measured may be any one or more of MBNL1, SOS1, PKM, zTTN, GOGLA4, and CLASP1. Splicing may be measured by any one or more techniques known in the art, e.g., RNA sequencing (RNA-seq), or platform technology such as the Nanostring platform.

As used herein, the term “sense” nucleic acid is a nucleic acid comprising a sequence that encodes all or a part of a transgene. In some examples, mRNA for a transgene is a sense nucleic acid.

As used herein, “antisense” nucleic acid is a sequence of nucleic acid that is complementary to a “sense” nucleic acid. For example, an antisense nucleic acid may be complementary to a mRNA encoding a transgene.

As used herein, the “guide region” of an RNAi is the strand of the RNAi that binds the target mRNA, typically on the basis of complementarity. The region of complementarity may encompass the all or a portion of the guide region. Typically, the region of complementarity includes at least the seed region. In many cases, the antisense region of a RNAi is the guide region.

As used herein, the “passenger region,” or “non-guide region,” used interchangeably herein, of an RNAi is the region of the RNAi that is complementary to the guide region. In many cases, the sense region of a RNAi is the passenger region.

As used herein, the “seed region” of a RNAi (e.g., miRNA) is a region of about 1-8 nucleotides in length of a microRNA. In some examples, the seed region and the 3′-UTR of its target mRNA may be a key determinant in RNAi recognition.

As used herein, “off-target gene silencing” refers to the pairing of a seed region of an RNAi with sequences in 3?-UTRs of unintended mRNAs and directs translational repression and destabilization of those transcripts (e.g., reduces expression of the unintended mRNAs).

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the invention described herein include “comprising.” “consisting,” and/or “consisting essentially of” aspects and embodiments.

### RNAi

In some aspects, the invention provides improved RNAi targeting DMPK RNA for the treatment of myotonic dystrophy type 1 (DM1). In some embodiments, the RNAi is a small inhibitory RNA (siRNA), a microRNA (miRNA), or a small hairpin RNA (shRNA). A small inhibitory or interfering RNA (siRNA) is known in the art as a double-stranded RNA molecule of approximately 19-25 (e.g., 19-23) base pairs in length that induces RNAi in a cell, miRNAs are typically smaller than siRNAs, can have multiple targets, and function to repress translation, degrade mRNA and in some instances cleaves mRNA endonucleolytically. A small hairpin RNA (shRNA) is known in the art as an RNA molecule comprising approximately 19-25 (e.g., 19-23) base pairs of double stranded RNA linked by a short loop (e.g., ˜4-11 nucleotides) that induces RNAi in a cell. In some embodiments, the RNAi comprises a first strand and a second strand, wherein a) the first strand and the second strand form a duplex: b) the first strand comprises a guide region, wherein the guide region comprises the nucleic acid sequence 5′-AGUCGAAGACAGUUCUAGGGU-3′ (SEQ ID NO: 1); and c) the second strand comprises a non-guide region. In some embodiments, the nucleic the guide region comprises the nucleic acid sequence 5′-AGUCGAAGACAGUUCUAGGGU-3′ (SEQ ID NO: 1) and the non-guide region comprises the sequence 5′-ACCCUAGAUGUCUUCGAUU-3′ (SEQ ID NO:2).

In some embodiments, the first strand comprises a guide region, wherein the guide region comprises a nucleic acid sequence having more than about 75%, 80%, 85%, 90%, 95%, 96%, 97%, 98%, 99% identity to 5′-AGUCGAAGACAGUUCUAGGGU-3′ (SEQ ID NO: 1). In some embodiments, the first strand comprises a guide region, wherein the guide region comprises a nucleic acid sequence having more than about 75%, 80%, 85%, 90%, 95%, 96%, 97%, 98%, 99% identity to 5′-AGUCGAAGACAGUUCUAGGGU-3′ (SEQ ID NO: 1) but maintains at least one CpG motif. In some embodiments, the second strand comprises a non-guide region, wherein the non-guide region comprises a nucleic acid sequence having more than about 75%, 80%, 85%, 90%, 95%, 96%, 97%, 98%, 99% identity to 5′-ACCCUAGAUGUCUUCGAUU-3′ (SEQ ID NO:2). In some embodiments, the second strand comprises a non-guide region, wherein the non-guide region comprises a nucleic acid sequence having more than about 75%, 80%, 85%, 90%, 95%, 96%, 97%, 98%, 99% identity to 5′-ACCCUAGAUGUCUUCGAUU-3′ (SEQ ID NO:2) but maintains at least one CpG motif.

In some embodiments, the RNAi comprises the nucleic acid sequence of SEQ ID NO: 7. In some embodiments, the RNAi comprises a nucleic acid sequence having more than about 75%, 80%, 85%, 90%, 95%, 96%, 97%, 98%, 99% identity to SEQ ID NO:7. In some embodiments, the RNAi comprises a nucleic acid sequence having more than about 75%, 80%, 85%, 90%, 95%, 96%, 97%, 98%, 99% identity to SEQ ID NO:7 but maintains at least one sequence (e.g., in a seed sequence).

In some embodiments, the invention provides a nucleic acid encoding an RNAi comprises a first strand and a second strand, wherein a) the first strand and the second strand form a duplex: b) the first strand comprises a guide region, and c) the second strand comprises a non-guide region. In some embodiments, the nucleic acid encoding the RNAi comprises the nucleic acid sequence of SEQ ID NO:4 and/or the nucleic acid of SEQ ID NO: 5. In some embodiments, the nucleic acid encoding the RNAi comprises a nucleic acid sequence having more than about 75%, 80%, 85%, 90%, 95%, 96%, 97%, 98%, 99% identity to SEQ ID NO:4 and/or a nucleic acid sequence having more than about 75%, 80%, 85%, 90%, 95%, 96%, 97%, 98%, 99% identity to SEQ ID NO:5. In some embodiments, the RNAi is encoded by the nucleic acid sequence of SEQ ID NO:8. In some embodiments, the RNAi is encoded by a nucleic acid sequence having more than about 75%, 80%, 85%, 90%, 95%, 96%, 97%, 98%, 99% identity to SEQ ID NO:8.

A microRNA (miRNA) is known in the art as an RNA molecule that induces RNAi in a cell comprising a short (e.g., 19-25 base pairs) sequence of double-stranded RNA linked by a loop and containing one or more additional sequences of double-stranded RNA comprising one or more bulges (e.g., mispaired or unpaired base pairs). As used herein, the term “miRNA” encompasses endogenous miRNAs as well as exogenous or heterologous miRNAs. In some embodiments, “miRNA” may refer to a pri-miRNA or a pre-miRNA. During miRNA processing, a pri-miRNA transcript is produced. The pri-miRNA is processed by Drosha-DGCR8 to produce a pre-miRNA by excising one or more sequences to leave a pre-miRNA with a 5′ flanking region, a guide strand, a loop region, a non-guide strand, and a 3′ flanking region: or a 5′ flanking region, a non-guide strand, a loop region, a guide strand, and a 3′ flanking region. The pre-miRNA is then exported to the cytoplasm and processed by Dicer to yield a siRNA with a guide strand and a non-guide (or passenger) strand. The guide strand is then used by the RISC complex to catalyze gene silencing, e.g., by recognizing a target RNA sequence complementary to the guide strand. Further description of miRNAs may be found, e.g., in WO 2008/150897. The recognition of a target sequence by a miRNA is primarily determined by pairing between the target and the miRNA seed sequence, e.g., nucleotides 1-8 (5′ to 3′) of the guide strand (see, e.g., Boudreau, R. L. et al. (2013) Nucleic Acids Res. 41:e9).

In the pri/pre-miRNA structure, the guide strand: non-guide strand interface in a duplex is formed in part through complementary base pairing (e.g., Watson-Crick base pairing). However, in some embodiments, this complementary base pairing does not extend through the entire duplex. In some embodiments, a bulge in the interface may exist at one or more nucleotide positions. As used herein, the term “bulge” may refer to a region of nucleic acid that is non-complementary to the nucleic acid opposite it in a duplex. In some embodiments, the bulge is formed when the regions of complementary nucleic acids bind to each other, whereas the regions of central non-complementary region do not bind. In some embodiments, the bulge is formed when the two strands of nucleic acid positioned between the two complementary regions are of different lengths. As described below, a bulge may comprise 1 or more nucleotides.

During miRNA processing, the miRNA is cleaved at a cleavage site adjacent to the guide strand: non-guide strand interface, thus releasing the siRNA duplex of the guide and non-guide strands. In some embodiments, the miRNA comprises a bulge in the sense or antisense strand adjacent to the cleavage site. To state another way, in some embodiments, the miRNA comprises a bulge in the guide or non-guide strand adjacent to the seed sequence.

In some embodiments, the miRNA comprises a bulge in the guide strand opposite the 5′ cleavage site of the mature non-guide strand. In some embodiments, the miRNA comprises a bulge opposite the 5′ nucleotide of the non-guide strand. In some embodiments, the miRNA comprises a bulge in the sense strand opposite the 3′ cleavage site of the mature guide strand. In some embodiments, the miRNA comprises a bulge opposite the 3′ nucleotide of the guide strand.

The safety of RNAi-based therapies can be hampered by the ability of small inhibitory RNAs (siRNAs) to bind to unintended mRNAs and reduce their expression, an effect known as off-target gene silencing. Off-targeting primarily occurs when the seed region (nucleotides 2-8 of the small RNA) pairs with sequences in 3′-UTRs of unintended mRNAs and directs translational repression and destabilization of those transcripts. Reduced off-targeting RNAi may be designed by substituting bases within the guide and nonguide sequences: e.g., by creating CpG motifs. Potential substitutions that may result in a significantly lower off-target score can be evaluated using the SiSPOTR algorithm, a specificity-focused siRNA design algorithm which identifies candidate sequences with minimal off-targeting potentials and potent silencing capacities (Boudreau et al, Nucleic Acids Res. 2013 January; 41 (1) e9. A reduced SiSPOTR score predicts sequences that have a lower number of potential human off targets compared parent RNAi molecules. In some embodiments of the invention, the RNAi is improved to reduce off-target gene silencing.

In some embodiments, the first strand and the second strand are linked by means of a RNA (e.g., a RNA linker) capable of forming a loop structure. As is commonly known in the art, an RNA loop structure (e.g., a stem-loop or hairpin) is formed when an RNA molecule comprises two sequences of RNA that basepair together separated by a sequence of RNA that does not base pair together. For example, a loop structure may form in the RNA molecule A-B-C if sequences A and C are complementary or partially complementary such that they base pair together, but the bases in sequence B do not base pair together.

In some embodiments, the RNA capable of forming a loop structure comprises from 4 to 50) nucleotides. In certain embodiments, the RNA capable of forming a loop structure comprises 13 nucleotides. In some embodiments, the number of nucleotides in the RNA capable of forming a loop is from 4 to 50 nucleotides or any integer therebetween. In some embodiments, from 0-50% of the loop can be complementary to another portion of the loop. As used herein, the term “loop structure” is a sequence that joins two complementary strands of nucleic acid. In some embodiments, 1-3 nucleotides of the loop structure are contiguous to the complementary strands of nucleic acid and may be complementary to 1-3 nucleotides of the distal portion of the loop structure. For example, the three nucleotides at the 5′ end of the loop structure may be complementary to the three nucleotides at the 3′ end of the loop structure.

In some embodiments, nucleic acid encoding an RNAi of the present disclosure comprises a heterologous miRNA scaffold. In some embodiments, use of a heterologous miRNA scaffold is used to modulate miRNA expression: for example, to increase miRNA expression or to decrease miRNA expression. Any miRNA scaffold known in the art may be used. In some embodiments, the miRNA scaffold is derived from a miR-155 scaffold (see, e.g., Lagos-Quintana, M. et al. (2002) Curr. Biol. 12:735-9 and the Invitrogen™ BLOCK-iT™ Pol II miR RNAi expression vector kit from Life Technologies, Thermo Fisher Scientific: Waltham, MA) or a mirGE scaffold (WO 2014/016817).

### Methods to Treat Myotonic Dystrophy Type-1 (DM-1)

Myotonic Dystrophy Type-1 (DM1) is a monogenic, autosomal-dominant, progressive disease caused by expansion of CTG repeats (>50) in the DMPK locus (dystrophia myotonica protein kinase). The DMPK with repeats are transcribed into mRNA, which forms hairpins and binds RNA binding proteins, sequestering them from their normal function. This leads to the appearance of nuclear foci, mis-splicing and ultimately myotonia. DM1 principally affects skeletal, cardiac and smooth muscle, resulting in significant physical, cognitive and behavioral impairments and disability.

In some aspects, the invention provides methods and compositions for treating myotonic dystrophy type 1 (DM1) in a mammal comprising administering to the mammal a pharmaceutical composition of the present disclosure (e.g., a pharmaceutical composition comprising a rAAV particle of the present disclosure). In some aspects, the invention provides methods and compositions for inhibiting the expression of DMPK in a mammal with DM-1 comprising administering to the mammal a pharmaceutical composition of the present disclosure (e.g., a pharmaceutical composition comprising a rAAV particle of the present disclosure). In some aspects, the invention provides methods and compositions for inhibiting the accumulation of DMPK in a cell of a mammal with DM1 comprising administering to the mammal a pharmaceutical composition of the present disclosure (e.g., a pharmaceutical composition comprising a rAAV particle of the present disclosure). In some aspects, the invention provides methods and compositions for ameliorating a symptom of DM1, comprising administration of an effective amount of rAAV particles comprising a vector encoding an RNAi of the present disclosure to the muscle brain of a mammal.

In some aspects, the invention provides an RNAi for targeting DMPK mRNA in a mammal with DM1. In some embodiments, the RNAi comprises a first strand comprising a first nucleic acid comprising the sequence 5′-AGUCGAAGACAGUUCUAGGGU-3′ (SEQ ID NO: 1) and a second strand comprising a second nucleic acid comprising the sequence 5′-ACCCUAGAUGUCUUCGAUU-3′ (SEQ ID NO:2). An RNAi described herein (e.g., as part of a rAAV vector) may find use, inter alia, in treating DM1.

In some embodiments, the RNAi is a small inhibitory RNA (siRNA), a microRNA (miRNA), or a small hairpin RNA (shRNA). A small inhibitory or interfering RNA (siRNA) is known in the art as a double-stranded RNA molecule of approximately 19-25 (e.g., 19-23) base pairs in length that induces RNAi in a cell. miRNAs are typically smaller than siRNAs, can have multiple targets, and function to repress translation, degrade mRNA and in some instances cleaves mRNA endonucleolytically. A small hairpin RNA (shRNA) is known in the art as an RNA molecule comprising approximately 19-25 (e.g., 19-23) base pairs of double stranded RNA linked by a short loop (e.g., ˜4-11 nucleotides) that induces RNAi in a cell.

In some embodiments, the miRNA comprises a guide sequence that is about 90% identical to SEQ ID NO:1. In some embodiments, the miRNA comprises a guide sequence that is about any of 90% identical, 91% identical, 92% identical, 93% identical, 94% identical, 95% identical, 96% identical, 97% identical, 98% identical, 99% identical, or 100% identical to SEQ ID NO:1.

In some embodiments, the miRNA comprises a non-guide sequence (passenger strand) that is about 90% identical to SEQ ID NO:2. In some embodiments, the miRNA comprises a non-guide sequence that is about any of 90% identical, 91% identical, 92% identical, 93% identical, 94% identical, 95% identical, 96% identical, 97% identical, 98% identical, 99% identical, or 100% identical to SEQ ID NO:2.

In some embodiments, the first strand and the second strand are linked by means of RNA capable of forming a loop structure. As is commonly known in the art, an RNA loop structure (e.g., a stem-loop or hairpin) is formed when an RNA molecule comprises two sequences of RNA that basepair together separated by a sequence of RNA that does not base pair together. For example, a loop structure may form in the RNA molecule A-B-C if sequences A and C are complementary or partially complementary such that they base pair together, but the bases in sequence B do not base pair together.

In some embodiments, the RNA capable of forming a loop structure comprises from 4 to 50 nucleotides. In certain embodiments, the RNA capable of forming a loop structure comprises 13 nucleotides. In certain embodiments, the RNA capable of forming a loop structure comprises the nucleotide sequence GUUUUGGCCACUGACUGAC (SEQ ID NO: 3). In some embodiments, the vector genome comprises a nucleotide sequence that is at least about any of 80%, 85%, 90%, 91%, 92%, 93%, 94%, 95%, 96%, 97%, 98%, or 99% identical to the sequence of SEQ ID NO:3.

In some aspects, the invention provides methods comprising administering to a mammal (e.g., a mammal with DM1) an RNAi comprising a first strand comprising a first nucleic acid comprising the sequence 5′-AGUCGAAGACAGUUCUAGGGU-3′ (SEQ ID NO: 1) and a second strand comprising a second nucleic acid comprising the sequence5′-ACCCUAGAUGUCUUCGAUU-3′ (SEQ ID NO:2). In some embodiments, a recombinant viral particle comprises the RNAi. In some embodiments, the recombinant viral particle is an AAV particle encapsidating a rAAV vector, wherein the rAAV vector encodes the RNAi.

In some embodiments, delivery of rAAV particles is by systemic injection of rAAV particles to the mammal. In some embodiments, the systemic injection is intravenous injection, intra-arterial injection, intramuscular injection, intraperitoneal injection, intradermal injection, or subcutaneous injection, intra-CSF and intrathecal administrations (IT).

In some aspects, the invention provides methods for treating DM1 in a mammal comprising administering to the mammal the pharmaceutical composition of the present disclosure. In some aspects, the invention provides methods for inhibiting the accumulation of DMPK in a cell of a mammal with DM1 comprising administering to the mammal the pharmaceutical composition of the present disclosure. In some aspects, the invention provides methods for inhibiting the expression of DMPK in a mammal with DM1 comprising administering to the mammal the pharmaceutical composition of the present disclosure. In some embodiments, the DMPK is a mutant DMPK (e.g., an DMPK comprising greater than 37 or greater than 50 CTG repeats).

In some embodiments, the invention provides a method for treating a human with DM1 by administering an effective amount of a pharmaceutical composition comprising a rAAV vector encoding an RNAi of the present disclosure to suppress the activity of a mutant DMPK. In some embodiments, the pharmaceutical composition comprises one or more pharmaceutically acceptable excipients.

In some embodiments, the methods comprise administering an effective amount of a pharmaceutical composition comprising a rAAV vector encoding an RNAi of the present disclosure to suppress the activity of a mutant DMPK. In some embodiments, the viral titer of the rAAV particles is at least about any of 5×10¹², 6×10¹², 7×10¹², 8×10¹², 9×10¹² 10×10¹², 11×10¹², 15×10¹², 20×10¹², 25×10¹², 30×10¹², or 50×10¹² genome copies/mL. In some embodiments, the viral titer of the rAAV particles is about any of 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some embodiments, the viral titer of the rAAV particles is about any of 5×10¹² to 10×10¹², 10×10¹² to 25×10¹², or 25×10¹² to 50×10¹² genome copies/mL. In some embodiments, the viral titer of the rAAV particles is at least about any of 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some embodiments, the viral titer of the rAAV particles is about any of 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL. In some embodiments, the viral titer of the rAAV particles is about any of 5×10⁹ to 10×10⁹, 10×10⁹ to 15×10⁹, 15×10⁹ to 25×10⁹, or 25×10⁹ to 50×10⁹ transducing units/mL. In some embodiments, the viral titer of the rAAV particles is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some embodiments, the viral titer of the rAAV particles is at least any of about 5×10¹⁰ to 6×10¹⁰, 6×10¹⁰ to 7×10¹⁰. 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some embodiments, the viral titer of the rAAV particles is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL.

In some embodiments, the dose concentration of rAAV particles administered to the individual is any of about 1×10⁸ to about 2×10¹³ genome copies/mL. In some embodiments, the dose concentration of rAAV particles administered to the individual is any of about 1×10⁸ to about 5×10⁸, about 5×10⁸ to about 10×10⁸, about 10×10⁸ to about 20×10⁸, about 20×10⁸ to about 30×10⁸, about 30×10⁸ to about 40×10⁸, about 40×10⁸ to about 50×10⁸, or about 50×10⁸ to about 100×10⁸ genome copies/mL. In some embodiments, the dose concentration of rAAV particles administered to the individual is any of about 1×10⁹ to about 5×10⁹, about 5×10⁹ to about 10×10⁹, about 10×10⁹ to about 20×10⁹, about 20×10⁹ to about 30×10⁹, about 30×10⁹ to about 40×10⁹, about 40×10⁹ to about 50×10⁹, or about 50×10⁹ to about 100×10⁹ genome copies/mL. In some embodiments, the dose concentration of rAAV particles administered to the individual is any of about 1×10¹⁰ to about 5×10¹⁰, about 5×10¹⁰ to about 10×10¹⁰, about 10×10¹⁰ to about 20×10¹⁰, about 20×10¹⁰ to about 30×10¹⁰, about 30×10¹⁰ to about 40×10¹⁰, about 40×10¹⁰ to about 50×10¹⁰, or about 50×10¹⁰ to about 100×10¹⁰ genome copies/mL. In some embodiments, the dose concentration of rAAV particles administered to the individual is any of about 1×10¹¹ to about 5×10¹¹, about 5×10¹¹ to about 10×10¹¹, about 10×10¹¹ to about 20×10¹¹, about 20×10¹¹ to about 30×10¹¹, about 30×10¹¹ to about 40×10¹¹, about 40×10¹¹ to about 50×10¹¹, or about 50×10¹¹ to about 100×10¹¹ genome copies/mL. In some embodiments, the dose concentration of rAAV particles administered to the individual is any of about 1×10¹² to about 5×10¹², about 5×10¹² to about 10×10¹², about 10×10¹² to about 20×10¹², about 20×10¹² to about 30×10¹², about 30×10¹² to about 40×10¹², about 40×10¹² to about 50×10¹², or about 50×10¹² to about 100×10¹² genome copies/mL. In some embodiments, the dose concentration of rAAV particles administered to the individual is any of about 1×10¹³ to about 2×10¹³ genome copies/mL. In some embodiments, the dose concentration of rAAV particles administered to the individual is about 1×10⁸, about 5×10⁸, about 1×10⁹, about 5×10⁹, about 1×10¹⁰, about 5×10¹⁰, about 1×10¹¹, about 5×10¹¹, about 1×10¹², about 5×10¹², about 1×10¹³, or about 2×10¹³ genome copies/mL. In some embodiments, the dose concentration of rAAV particles administered to the individual is about 5×10¹² genome copies/mL. In some embodiments, the dose concentration of rAAV particles administered to the individual is about 1×10¹³ genome copies/mL. In some embodiments, the dose concentration of rAAV particles administered to the individual is about 2×10¹³ genome copies/mL.

In some embodiments, the dose of rAAV particles administered to the individual is at least about any of 1×10⁸ to about 2×10¹⁴ genome copies/kg of body weight. In some embodiments, the dose of rAAV particles administered to the individual is between about any of 1×10⁸ to about 2×10¹⁴ genome copies/kg of body weight. In some embodiments, the dose of rAAV particles administered to the individual is between any of about 1×10⁸ to about 1×10¹⁴, 5×10⁸ to about 1×10¹⁴, 1×10⁹ to about 1×10¹⁴, 5×10⁹ to about 1×10¹⁴. 1×10¹⁰ to about 1×10¹⁴, 5×10¹⁰ to about 1×10¹⁴, 1×10¹¹ to about 1×10¹⁴, 5×10¹¹ to about 1×10¹⁴, 1×10¹² to about 1×10¹⁴, 5×10¹² to about 1×10¹⁴, 1×10¹³ to about 1×10¹⁴, 5×10¹³ to about 1×10¹⁴, 1×10⁸ to about 5×10¹³, 5×10⁸ to about 5×10¹³, 1×10⁹ to about 5×10¹³, 5×10⁹ to about 5×10¹³, 1×10¹⁰ to about 5×10¹³, 5×10¹⁰ to about 5×10¹³, 1×10¹¹ to about 5×10¹³, 5×10¹¹ to about 5×10¹³, 1×10¹² to about 5×10¹³, 5×10¹² to about 5×10¹³, 1×10¹³ to about 5×10¹³, 1×10⁸ to about 1×10¹³, 5×10⁸ to about 1×10¹³, 1×10⁹ to about 1×10¹³, 5×10⁹ to about 1×10¹³, 1×10¹⁰ to about 1×10¹³, 5×10¹⁰ to about 1×10¹³, 1×10¹¹ to about 1×10¹³, 5×10¹¹ to about 1×10¹³, 1×10¹² to about 1×10¹³, 5×10¹² to about 1×10¹³, 1×10⁸ to about 5×10¹², 5×10⁸ to about 5×10¹², 1×10⁹ to about 5×10¹², 5×10⁹ to about 5×10¹², 1×10¹⁰ to about 5×10¹², 5×10¹⁰ to about 5×10¹², 1×10¹¹ to about 5×10¹², 5×10¹¹ to about 5×10¹², 1×10¹² to about 5×10¹², 1×10⁸ to about 1×10¹², 5×10⁸ to about 1×10¹², 1×10⁹ to about 1×10¹², 5×10⁹ to about 1×10¹², 1×10¹⁰ to about 1×10¹², 5×10¹⁰ to about 1×10¹², 1×10¹¹ to about 1×10¹², 5×10¹¹ to about 1×10¹², 1×10⁸ to about 5×10¹¹, 5×10⁸ to about 5×10¹¹, 1×10⁹ to about 5×10¹¹, 5×10⁹ to about 5×10¹¹, 1×10¹⁰ to about 5×10¹¹, 5×10¹⁰ to about 5×10¹¹, 1×10¹¹ to about 5×10¹¹, 1×10⁸ to about 1×10¹¹, 5×10⁸ to about 1×10¹¹, 1×10⁹ to about 1×10¹¹, 5×10⁹ to about 1×10¹¹, 1×10¹⁰ to about 1×10¹¹, 5×10¹⁰ to about 1×10¹¹, 1×10⁸ to about 5×10¹⁰, 5×10⁸ to about 5×10¹⁰, 1×10⁹ to about 5×10¹⁰, 5×10⁹ to about 5×10¹⁰, 1×10¹⁰ to about 5×10¹⁰, 1×10⁸ to about 1×10¹⁰, 5×10⁸ to about 1×10¹⁰, 1×10⁹ to about 1×10¹⁰, 5×10⁹ to about 1×10¹⁰, 1×10⁸ to about 5×10⁹, 5×10⁸ to about 5×10⁹, 1×10⁹ to about 5×10⁹, 1×10⁸ to about 1×10⁹, 5×10⁸ to about 1×10⁹, or 1×10⁸ to about 5× 10⁸, gc/kg body weight. In some embodiments, the dose of rAAV particles administered to the individual is about 1×10⁹, about 5×10⁹, about 1×10¹⁰, about 5×10¹⁰, about 1×10¹¹ about 5×10¹¹, about 1×10¹², about 5×10¹², about 1×10¹³, about 5×10¹³, about 1×10¹⁴, or about 2×10¹⁴ genome copies/kg body weight. In some embodiments, the dose of rAAV particles administered to the individual is about 5×10¹³ genome copies/kg body weight. In some embodiments, the dose of rAAV particles administered to the individual is about 1×10¹⁴ genome copies/kg body weight. In some embodiments, the dose of rAAV particles administered to the individual is about 2×10¹⁴ genome copies/kg body weight.

In some embodiments, the total amount of rAAV particles administered to the individual is at least about any of 1×10⁹ to about 2×10¹⁴ genome copies/kg body weight. In some embodiments, the total amount of rAAV particles administered to the individual is about any of 1×10⁹ to about 2×10¹⁴ genome copies/kg body weight. In some embodiments of the invention, the volume of the composition injected to the striatum is more than about any one of 10 μl, 25 μl, 50 μl, 75 μl, 100 μl, 200 μl, 300 μl, 400 μl, 500 μl, 600 μl, 700 μl, 800 μl, 900 μl, 1 mL, 5 mL, 10 mL, 25 mL, 50 mL, 75 mL, or 100 mL or any amount therebetween.

Compositions of the invention (e.g., rAAV particles comprising a vector encoding an RNAi of the present disclosure) can be used either alone or in combination with one or more additional therapeutic agents for treating DM1. The interval between sequential administration can be in terms of at least (or, alternatively, less than) minutes, hours, or days.

In some embodiments, the RNAi to treat DM1 is administered in combination with an immunosuppressive agent: for example, to suppress an immune response to the RNAi. In some embodiments, the immunosuppressive agent is administered before administration of the RNAi. In some embodiments, the immunosuppressive agent is administered at the same time as administration of the RNAi. In some embodiments, the immunosuppressive agent is administered after administration of the RNAi. In some embodiments, the immunosuppressive agent is administered in any combination of before, during or after administration of the RNAi.

In some embodiments, the rAAV particles to treat DM1 are administered in combination with an immunosuppressive agent: for example, to suppress an immune response to the rAAV particle and/or to the transgene product of the rAAV particle. In some embodiments, the immunosuppressive agent is administered before administration of the rAAV particle. In some embodiments, the immunosuppressive agent is administered at the same time as administration of the rAAV particle. In some embodiments, the immunosuppressive agent is administered after administration of the rAAV particle. In some embodiments, the immunosuppressive agent is administered in any combination of before, during or after administration of the rAAV particle.

In some embodiments, the invention provides the use of an effective amount of any of the RNAi described herein in the manufacture of a medicament for treating myotonic dystrophy-1 (DM1) in a mammal in need thereof. In some embodiments, the invention provides the use of an effective amount of any of the RNAi described herein in the manufacture of a medicament for inhibiting the expression of dystrophia myotonica protein kinase (DMPK) in a mammal with DM-1 in need thereof. In some embodiments, the invention provides the use of an effective amount of any of the RNAi described herein in the manufacture of a medicament for inhibiting the accumulation of DMPK RNA in a cell of a mammal with DM-1 in need thereof.

In some embodiments, the invention provides the use of an effective amount of any of the RNAi described herein for treating myotonic dystrophy-1 (DM1) in a mammal in need thereof. In some embodiments, the invention provides the use of an effective amount of any of the RNAi described herein for inhibiting the expression of dystrophia myotonica protein kinase (DMPK) in a mammal with DM-1 in need thereof. In some embodiments, the invention provides the use of an effective amount of any of the RNAi described herein for inhibiting the accumulation of DMPK RNA in a cell of a mammal with DM-1 in need thereof.

In some embodiments, the invention provides an RNAi described herein for treating myotonic dystrophy-1 (DM1) in a mammal in need thereof. In some embodiments, the invention provides any of the RNAi described herein for inhibiting the expression of dystrophia myotonica protein kinase (DMPK) in a mammal with DM-1 in need thereof. In some embodiments, the invention provides any of the RNAi described herein for inhibiting the accumulation of DMPK RNA in a cell of a mammal with DM-1 in need thereof.

In some embodiments, the invention provides the use of an effective amount of any of the viral particles (e.g., AAV particles) described herein in the manufacture of a medicament for treating myotonic dystrophy-1 (DM1) in a mammal in need thereof. In some embodiments, the invention provides the use of an effective amount of any of the viral particles (e.g., AAV particles) described herein in the manufacture of a medicament for inhibiting the expression of dystrophia myotonica protein kinase (DMPK) in a mammal with DM-1 in need thereof. In some embodiments, the invention provides the use of an effective amount of any of the viral particles (e.g., AAV particles) described herein in the manufacture of a medicament for inhibiting the accumulation of DMPK RNA in a cell of a mammal with DM-1 in need thereof.

In some embodiments, the invention provides the use of an effective amount of any of the viral particles (e.g., AAV particles) described herein for treating myotonic dystrophy-1 (DM1) in a mammal in need thereof. In some embodiments, the invention provides the use of an effective amount of any of the viral particles (e.g., AAV particles) described herein for inhibiting the expression of dystrophia myotonica protein kinase (DMPK) in a mammal with DM-1 in need thereof. In some embodiments, the invention provides the use of an effective amount of any of the viral particles (e.g., AAV particles) described herein for inhibiting the accumulation of DMPK RNA in a cell of a mammal with DM-1 in need thereof.

In some embodiments, the invention provides viral particles (e.g., AAV particles) described herein for treating myotonic dystrophy-1 (DM1) in a mammal in need thereof. In some embodiments, the invention provides any of the viral particles (e.g., AAV particles) described herein for inhibiting the expression of dystrophia myotonica protein kinase (DMPK) in a mammal with DM-1 in need thereof. In some embodiments, the invention provides any of the viral particles (e.g., AAV particles) described herein for inhibiting the accumulation of DMPK RNA in a cell of a mammal with DM-1 in need thereof.

In some embodiments, the invention provides the use of an effective amount of any of the compositions described herein in the manufacture of a medicament for treating myotonic dystrophy-1 (DM1) in a mammal in need thereof. In some embodiments, the invention provides the use of an effective amount of any of the compositions described herein in the manufacture of a medicament for inhibiting the expression of dystrophia myotonica protein kinase (DMPK) in a mammal with DM-1 in need thereof. In some embodiments, the invention provides the use of an effective amount of any of the compositions described herein in the manufacture of a medicament for inhibiting the accumulation of DMPK RNA in a cell of a mammal with DM-1 in need thereof.

In some embodiments, the invention provides the use of an effective amount of any of the compositions described herein for treating myotonic dystrophy-1 (DM1) in a mammal in need thereof. In some embodiments, the invention provides the use of an effective amount of any of the compositions described herein for inhibiting the expression of dystrophia myotonica protein kinase (DMPK) in a mammal with DM-1 in need thereof. In some embodiments, the invention provides the use of an effective amount of any of the compositions described herein for inhibiting the accumulation of DMPK RNA in a cell of a mammal with DM-1 in need thereof.

In some embodiments, the invention provides compositions described herein for treating myotonic dystrophy-1 (DM1) in a mammal in need thereof. In some embodiments, the invention provides any of the compositions described herein for inhibiting the expression of dystrophia myotonica protein kinase (DMPK) in a mammal with DM-1 in need thereof. In some embodiments, the invention provides any of the compositions described herein for inhibiting the accumulation of DMPK RNA in a cell of a mammal with DM-1 in need thereof.

### RNAi Expression Constructs and Vectors

The invention provides expression constructs, vectors and rAAV particles for expression of the RNAi described herein.

In some embodiments, nucleic acid encoding an RNAi of the present disclosure comprises a heterologous miRNA scaffold. In some embodiments, use of a heterologous miRNA scaffold is used to modulate miRNA expression: for example, to increase miRNA expression or to decrease miRNA expression. Any miRNA scaffold known in the art may be used. In some embodiments, the miRNA scaffold is derived from a miR-155 scaffold (see, e.g., Lagos-Quintana, M. et al. (2002) Curr. Biol. 12:735-9 and the Invitrogen™ BLOCK-iT™ Pol II miR RNAi expression vector kit from Life Technologies, Thermo Fisher Scientific: Waltham, MA) or the mirGE scaffold (WO 2014/016817). In some embodiments, nucleic acid encoding an RNAi of the present disclosure comprises a miRNA scaffold. In some embodiments, miRNA scaffold is provided by SEQ ID NO:11. In some embodiments, the miRNA scaffold comprises a nucleic acid with greater than 80%, 85%, 90%, 95%, or 99% identity to the nucleic acid sequence of SEQ ID NO:11.

In some embodiments, the RNAi targets RNA encoding a polypeptide associated with DM1 (e.g., mutant DMPK). Without wishing to be bound to theory, it is thought that an RNAi may be used to reduce or eliminate the expression and/or activity of a polypeptide whose gain-of-function has been associated with DM1 (e.g., mutant DMPK).

In some embodiments, the transgene (e.g., encoding an RNAi of the present disclosure) is operably linked to a promoter. Exemplary promoters include, but are not limited to, the cytomegalovirus (CMV) immediate early promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter: the cytomegalovirus enhancer/chicken beta-actin/Rabbit β-globin promoter (CAG promoter: Niwa et al., Gene, 1991, 108 (2): 193-9) and the elongation factor 1-alpha promoter (EF1-alpha) promoter (Kim et al., Gene, 1990, 91 (2): 217-23 and Guo et al., Gene Ther., 1996, 3 (9): 802-10). In some embodiments, the promoter comprises a human β-glucuronidase promoter or a cytomegalovirus enhancer linked to a chicken β-actin (CBA) promoter. The promoter can be a constitutive, inducible or repressible promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088): the ecdysone insect promoter (No et al, Proc. Natl. Acad. Sci. USA, 93:3346-3351 (1996)), the tetracycline-repressible system (Gossen et al. Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a muscle-specific promoter. In some embodiments, the promoter is a desmin promoter. In some embodiments, the promoter is a human desmin promoter (e.g., −228 to +75 of the human desmin gene: e.g., SEQ ID NO:23). In some embodiments, the promoter is a modified desmin promoter. In some embodiments, the desmin promoter comprises desmin promoter elements important for high level expression in muscle cells (Li and Paulin, et. al. 1991. Journal of Biol Chem). In some embodiments, the desmin promoter comprises at least one copy of the Byrne desmin enhancer (e.g., SEQ ID NO: 21). In some embodiments, the desmin promoter comprises at least one copy of the Paulin desmin enhancer (−973 to −693) (e.g., SEQ ID NO:22). In some embodiments, the desmin promoter comprises one copy of Byrne desmin enhance are one copy of the Paulin desmin enhancer (−973 to −693). In some embodiments, the desmin promoter comprises one copy of Byrne desmin enhance are one copy of the Paulin desmin enhancer (−973 to −693) and the promoter of the human desmin gene (−228 to +75).

In some aspects, the invention provides an expression cassette (e.g., and expression cassette for expression of a transgene (e.g., a therapeutic transgene) in a muscle cell), wherein the expression cassette comprises a modified desmin promoter, wherein the desmin promoter comprises one or more enhancer elements and the promoter for the human desmin gene. In some embodiments, the desmin promoter comprises two enhancer elements and the promoter for the human desmin gene. In some embodiments, the desmin promoter comprises one or more Byrne enhancer element and/or one or more Paulin enhancer elements. In some embodiments, the desmin promoter comprises one or more enhancer element comprising the nucleotide sequence of SEQ ID NO:21, In some embodiments, the desmin promoter comprises one or more enhancer element comprising the nucleotide sequence of SEQ ID NO:22. In some embodiments, the desmin promoter comprises one or more enhancer element comprising the nucleotide sequence of SEQ ID NO:21 and one or more enhancer element comprising the nucleotide sequence of SEQ ID NO:22. In some embodiments, the desmin promoter comprises one or more enhancer element comprising a nucleotide sequence having at least about any of 80%, 85%, 90%, 95%, or 99% identity to the nucleotide sequence of SEQ ID NO:21. In some embodiments, the desmin promoter comprises one or more enhancer element comprising a nucleotide sequence having at least about any of 80%, 85%, 90%, 95%, or 99% identity to the nucleotide sequence of SEQ ID NO: 22. In some embodiments, the desmin promoter comprises one or more enhancer element comprising a nucleotide sequence having at least about any of 80%, 85%, 90%, 95%, or 99% identity to the nucleotide sequence of SEQ ID NO:21 and one or more enhancer element comprising a nucleotide sequence having at least about any of 80%, 85%, 90%, 95%, or 99% identity to the nucleotide sequence of SEQ ID NO:22.

In some embodiments, the expression cassette comprising the modified desmin promoter further comprises an intron. In some embodiments, the intron is a rabbit β-globin intron. In some embodiments, the intron comprises the nucleotide sequence of SEQ ID NO: 13. In some embodiments, the intron comprises a nucleotide sequence having at least about any of 80%, 85%, 90%, 95%, or 99% identity to the nucleotide sequence of SEQ ID NO: 13. In some embodiments, the nucleic acid encoding the transgene (e.g., a therapeutic transgene) is embedded in the intron. In some embodiments, the intron comprises a 5′ arm and a 3′ arm, wherein the 5′ arm is located 5′ to the nucleic acid encoding the transgene and the 3′ arm is located 3′ to the nucleic acid encoding the transgene. In some embodiments, the 5′ arm of the intron comprises nucleic acid with the sequence of SEQ ID NO:14. In some embodiments, the 5′ arm of the intron comprises nucleic acid with a sequence having at least about any of 80%, 85%, 90%, 95%, or 99% identity to the nucleotide sequence of SEQ ID NO: 14. In some embodiments, the 3′ arm of the intron comprises nucleic acid with the sequence of SEQ ID NO:15. In some embodiments, the 3′ arm of the intron comprises nucleic acid with a sequence having at least about any of 80%, 85%, 90%, 95%, or 99% identity to the nucleotide sequence of SEQ ID NO: 15. In some embodiments, the 5′ arm of the intron comprises nucleic acid with the sequence of SEQ ID NO: 14 and the 3′ arm of the intron comprises nucleic acid with the sequence of SEQ ID NO:15. In some embodiments, the 5′ arm of the intron comprises nucleic acid with a sequence having at least about any of 80%, 85%, 90%, 95%, or 99% identity to the nucleotide sequence of SEQ ID NO: 14 and the 3′ arm of the intron comprises nucleic acid with a sequence having at least about any of 80%, 85%, 90%, 95%, or 99% identity to the nucleotide sequence of SEQ ID NO:15.

In some embodiments, the expression cassette comprising the modified desmin promoter further comprises a polyadenylation signal. In some embodiments, the polyadenylation signal is a bovine growth hormone polyadenylation signal, an SV40 polyadenylation signal, or a HSV TK pA. In some embodiments, the polyadenylation signal is a minimal bovine growth hormone polyadenylation signal. In some embodiments, the bovine growth hormone polyadenylation signal comprises nucleic acid with the sequence of SEQ ID NO:16. In some embodiments, the bovine growth hormone polyadenylation signal comprises nucleic acid with a sequence having at least about any of 80%, 85%, 90%, 95%, or 99% identity to the nucleotide sequence of SEQ ID NO:16.

In some embodiments, the invention provides an expression cassette comprising a modified desmin promoter for expression of a transgene (e.g., a therapeutic transgene) in a muscle cell. In some embodiments, the transgene encodes a polypeptide (e.g., a therapeutic polypeptide). In some embodiments, the transgene encodes a nucleic acid (e.g., a therapeutic nucleic acid). In some embodiments, the transgene encodes an RNAi. In some embodiments, the transgene encodes an siRNA, an shRNA, or an miRNA.

In some aspects, the invention provides a modified desmin promoter, wherein the desmin promoter comprises one or more enhancer elements and the promoter for the human desmin gene. In some embodiments, the desmin promoter comprises two enhancer elements and the promoter for the human desmin gene. In some embodiments, the desmin promoter comprises one or more Byrne enhancer element and/or one or more Paulin enhancer elements. In some embodiments, the desmin promoter comprises one or more enhancer element comprising the nucleotide sequence of SEQ ID NO:21, In some embodiments, the desmin promoter comprises one or more enhancer element comprising the nucleotide sequence of SEQ ID NO:22. In some embodiments, the desmin promoter comprises one or more enhancer element comprising the nucleotide sequence of SEQ ID NO:21 and one or more enhancer element comprising the nucleotide sequence of SEQ ID NO:22. In some embodiments, the desmin promoter comprises one or more enhancer element comprising a nucleotide sequence having at least about any of 80%, 85%, 90%, 95%, or 99% identity to the nucleotide sequence of SEQ ID NO:21. In some embodiments, the desmin promoter comprises one or more enhancer element comprising a nucleotide sequence having at least about any of 80%, 85%, 90%, 95%, or 99% identity to the nucleotide sequence of SEQ ID NO: 22. In some embodiments, the desmin promoter comprises one or more enhancer element comprising a nucleotide sequence having at least about any of 80%, 85%, 90%, 95%, or 99% identity to the nucleotide sequence of SEQ ID NO:21 and one or more enhancer element comprising a nucleotide sequence having at least about any of 80%, 85%, 90%, 95%, or 99% identity to the nucleotide sequence of SEQ ID NO:22.

In some aspects, the invention provides rAAV particles comprising a recombinant self-complementing genome (e.g., a self-complementary rAAV vector). AAV viral particles with self-complementing vector genomes and methods of use of self-complementing AAV genomes are described in U.S. Pat. Nos. 6,596,535; 7,125,717; 7,465,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a heterologous nucleic acid). In some embodiments, the vector comprises first nucleic acid sequence encoding the heterologous nucleic acid and a second nucleic acid sequence encoding a complement of the nucleic acid, where the first nucleic acid sequence can form intrastrand base pairs with the second nucleic acid sequence along most or all of its length.

In some embodiments, the first heterologous nucleic acid sequence encoding a RNAi and a second heterologous nucleic acid sequence encoding the complement of the RNAi are linked by a mutated ITR (e.g., the right ITR). In some embodiments, the ITR comprises the polynucleotide sequence 5′-CCACTCCCTCTCTGCGCGCTCGCTCGCTCACTGAGGCCGGGCGACCAAAGGTCGC CCGACGCCCGGGCTTTGCCCGGGCGGCCTCAGTGAGCGAGCGAGCGCGCAGAGA GGGA-3 (SEQ ID NO:27). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

rAAV Particles and Methods of Producing rAAV Particles

The invention provides rAAV particles comprising the RNAi as disclosed herein. In some embodiments, the invention provides methods of using recombinant viral particles to deliver RNAi to treat a DM1. In some embodiments, the rAAV particle comprises a sequence encoding the RNAi of the present disclosure flanked by one or two ITRs. The nucleic acid is encapsidated in the AAV particle. The AAV particle also comprises capsid proteins. In some embodiments, the nucleic acid comprises the coding sequence(s) of interest (e.g., nucleic acid encoding the RNAi of the present disclosure) operatively linked components in the direction of transcription, control sequences including transcription initiation and termination sequences, thereby forming an expression cassette. The expression cassette is flanked on the 5′ and 3′ end by at least one functional AAV ITR sequences. By “functional AAV ITR sequences” it is meant that the ITR sequences function as intended for the rescue, replication and packaging of the AAV virion. See Davidson et al., PNAS, 2000, 97 (7) 3428-32; Passini et al., J. Virol., 2003, 77 (12): 7034-40; and Pechan et al., Gene Ther., 2009, 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99 (18): 11854-6; Gao et al., PNAS, 2003, 100 (10): 6081-6; and Bossis et al., J. Virol., 2003, 77 (12): 6799-810. Use of any AAV serotype is considered within the scope of the present invention. In some embodiments, a rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAVrh74, AAV DJ, a goat AAV, bovine AAV, or mouse AAV capsid serotype ITRs or the like. In some embodiments, the nucleic acid in the AAV comprises an ITR of AAV1, AAV2, AAV3, AAV4, AAV5, AAV6, AAV7, AAV8, AAVrh8, AAVrh8R, AAV9, AAV10, AAVrh10, AAV11, AAV12, AAVrh74, AAV DJ, a goat AAV, bovine AAV, or mouse AAV capsid serotype ITRs or the like. In some embodiments, the nucleic acid in the AAV further encodes an RNAi as described herein. For example, the nucleic acid in the AAV can comprise at least one ITR of any AAV serotype contemplated herein and can further encode an RNAi comprising one strand that comprises a guide region and another strand that comprises a non-guide region. In one embodiment, the nucleic acid in the AAV can comprise at least one ITR of any AAV serotype and can further encode an RNAi comprising a first strand comprising a first nucleic acid comprising the sequence 5′-AGUCGAAGACAGUUCUAGGGU-3′ (SEQ ID NO:1) or a sequence with 80%, 85%, 90%, or 95% identity to SEQ ID NO:1, and a second strand comprising a second nucleic acid comprising the sequence 5′-ACCCUAGAUGUCUUCGAUU-3′ (SEQ ID NO:2) or a sequence with 80%, 85%, 90%, or 95% identity to SEQ ID NO:2.

In some embodiments, the nucleic acid in the AAV comprises 5′ to 3′ nucleic acid encoding the following: an ITR (e.g., an AAV2 ITR), a promoter, a nucleic acid encoding an RNAi as disclosed herein, a polyadenylation signal, and an AAV ITR (e.g., an AAV2 ITR). In some embodiments, the nucleic acid in the AAV comprises 5′ to 3′ nucleic acid encoding the following: an ITR (e.g., an AAV2 ITR), a promoter, a nucleic acid encoding an RNAi comprising a first strand comprising a first nucleic acid comprising the sequence 5′-AGUCGAAGACAGUUCUAGGGU-3′ (SEQ ID NO:1) or a sequence with 80%, 85%, 90%, or 95% identity to SEQ ID NO:1, and a second strand comprising a second nucleic acid comprising the sequence 5′-ACCCUAGAUGUCUUCGAUU-3′ (SEQ ID NO:2) or a sequence with 80%, 85%, 90%, or 95% identity to SEQ ID NO:2, a polyadenylation signal, and an AAV ITR (e.g., an AAV2 ITR). In some embodiments, the nucleic acid in the AAV comprises 5′ to 3′ nucleic acid encoding the following: an ITR (e.g., an AAV2 ITR), a desmin promoter, a nucleic acid encoding an RNAi as disclosed herein, a polyadenylation signal (e.g., a bovine growth hormone polyA), and an AAV ITR (e.g., an AAV2 ITR). In some embodiments, the nucleic acid in the AAV comprises 5′ to 3′ nucleic acid encoding the following: all or a functional portion of an ITR (e.g., an AAV2 ITR), a desmin promoter, an intron (e.g., a chimeric intron), a nucleic acid encoding an RNAi comprising a first strand comprising a first nucleic acid comprising the sequence 5′-AGUCGAAGACAGUUCUAGGGU-3′ (SEQ ID NO:1) or a sequence with 80%, 85%, 90%, or 95% identity to SEQ ID NO:1, and a second strand comprising a second nucleic acid comprising the sequence 5′-ACCCUAGAUGUCUUCGAUU-3′ (SEQ ID NO:2) or a sequence with 80%, 85%, 90%, or 95% identity to SEQ ID NO:2, a polyadenylation signal (e.g., a bovine growth hormone polyA), and an AAV ITR (e.g., an AAV2 ITR). In some embodiments, the first strand and second strand form a duplex. In some embodiments, the first strand is linked to the second strand by a linker. In some embodiments, the linker comprises the nucleic acid sequence of SEQ ID NO:3 or a sequence with 80%, 85%, 90%, or 95% identity to SEQ ID NO:3.

In some embodiments, the nucleic acid in the AAV comprises 5′ to 3′ nucleic acid encoding the following: all or a functional portion of an ITR (e.g., an AAV2 ITR), a stuffer sequence (e.g., all or a portion of a human alpha-1-antitrypsin (AAT) stuffer sequence), a desmin promoter, a 5′ arm of an intron (e.g., a rabbit β-globin intron), a nucleic acid encoding an RNAi comprising a first strand comprising a first nucleic acid comprising the sequence 5′-AGUCGAAGACAGUUCUAGGGU-3′ (SEQ ID NO:1) or a sequence with 80%, 85%, 90%, or 95% identity to SEQ ID NO:1, and a second strand comprising a second nucleic acid comprising the sequence 5′-ACCCUAGAUGUCUUCGAUU-3′ (SEQ ID NO: 2) or a sequence with 80%, 85%, 90%, or 95% identity to SEQ ID NO:2, a 3′ arm of an intron (e.g., a rabbit β-globin intron), a polyadenylation signal (e.g., a bovine growth hormone polyA)), a stuffer sequence (e.g., all or a portion of a human alpha-1-antitrypsin (AAT) stuffer sequence) and an AAV ITR (e.g., an AAV2 ITR).

In some embodiments, the nucleic acid in the AAV comprises 5′ to 3′ nucleic acid encoding the following: an ITR (e.g., an AAV2 ITR), a desmin promoter, a nucleic acid encoding an RNAi comprising a first strand comprising a first nucleic acid comprising the sequence 5′-ACCCUAGAUGUCUUCGAUU-3′ (SEQ ID NO:2) or a sequence with 80%, 85%, 90%, or 95% identity to SEQ ID NO:2, and a second strand comprising a second nucleic acid comprising the sequence 5′-ACCCUAGAUGUCUUCGAUU-3′ (SEQ ID NO: 1) or a sequence with 80%, 85%, 90%, or 95% identity to SEQ ID NO:1, a polyadenylation signal (e.g., a bovine growth hormone polyA), and an AAV ITR (e.g., an AAV2 ITR). In some embodiments, the first strand and second strand form a duplex. In some embodiments, the first strand is linked to the second strand by a linker. In some embodiments, the linker comprises the nucleic acid sequence of SEQ ID NO:6.

In some embodiments, the nucleic acid in the AAV comprises 5′ to 3′ nucleic acid encoding the following: all or a functional portion of an ITR (e.g., an AAV2 ITR), a stuffer sequence (e.g., all or a portion of a human alpha-1-antitrypsin (AAT) stuffer sequence), a desmin promoter, a 5′ arm of an intron (e.g., a rabbit β-globin intron), a nucleic acid encoding an RNAi comprising a first strand comprising a first nucleic acid comprising the sequence 5′-ACCCUAGAUGUCUUCGAUU-3′ (SEQ ID NO:2), and a second strand comprising a second nucleic acid comprising the sequence 5′-AGUCGAAGACAGUUCUAGGGU-3′ (SEQ ID NO:1), a 3′ arm of an intron (e.g., a rabbit β-globin intron), a polyadenylation signal (e.g., a bovine growth hormone polyA)), a stuffer sequence (e.g., all or a portion of a human alpha-1-antitrypsin (AAT) stuffer sequence) and an AAV ITR (e.g., an AAV2 ITR).

In some embodiments, a vector may include a (one or more) stuffer nucleic acid. In some embodiments, the stuffer nucleic acid may comprise a sequence that encodes a reporter polypeptide. As will be appreciated by those of skill in the art, the stuffer nucleic acid may be located in a variety of regions within the vector, and may be comprised of a continuous sequence (e.g., a single stuffer nucleic acid in a single location) or multiple sequences (e.g., more than one stuffer nucleic acid in more than one location (e.g., 2 locations, 3 locations, etc.) within the vector. In some embodiments, the stuffer nucleic acid may be located downstream of the RNAi sequence. In embodiments, the stuffer nucleic acid may be located upstream of the RNAi sequence (e.g., between the promoter and the nucleic acid encoding the RNAi). As will also be appreciated by those of skill in the art a variety of nucleic acids may be used as a stuffer nucleic acid. In some embodiments, the stuffer nucleic acid comprises all or a portion of a human alpha-1-antitrypsin (AAT) stuffer sequence or a C16 P1 chromosome 16 P1 clone (human C16) stuffer sequence. In some embodiments, the stuffer sequence comprises all or a portion of a gene. For example, the stuffer sequence comprises a portion of the human AAT sequence. One skilled in the art would recognize that different portions of a gene (e.g., the human AAT sequence) can be used as a stuffer fragment. For example, the stuffer fragment may be from the 5′ end of the gene, the 3′ end of the gene, the middle of a gene, a non-coding portion of the gene (e.g., an intron), a coding region of the gene (e.g. an exon), or a mixture of non-coding and coding portions of a gene. One skilled in the art would also recognize that all or a portion of stuffer sequence may be used as a stuffer sequence. In some embodiments, the vector comprises a 5′ stuffer sequence comprising the nucleotide sequence of SEQ ID NO: 18 or a nucleotide sequence with greater than about 80%, 85%, 90%, 95%, or 99% identity to the nucleotide sequence of SEQ ID NO: 18. In some embodiments, the vector comprises a 3′ stuffer sequence comprising the nucleotide sequence of SEQ ID NO:19 or a nucleotide sequence with greater than about 80%, 85%, 90%, 95%, or 99% identity to the nucleotide sequence of SEQ ID NO:19. In some embodiments, the vector comprises a 5′ stuffer sequence comprising the nucleotide sequence of SEQ ID NO:18 or a nucleotide sequence with greater than about 80%, 85%, 90%, 95%, or 99% identity to the nucleotide sequence of SEQ ID NO: 18 and comprises a 3′ stuffer sequence comprising the nucleotide sequence of SEQ ID NO: 19 or a nucleotide sequence with greater than about 80%, 85%, 90%, 95%, or 99% identity to the nucleotide sequence of SEQ ID NO:19.

In further embodiments, the rAAV particle comprises a capsid protein comprising capsid proteins of AAVrh74 or variants thereof. In some embodiments, the AAVrh74 capsid is a variant capsid. In some embodiments, the AAVrh74 variant capsid protein maintains the ability to form an AAV capsid. In some embodiments, the AAVrh74 variant capsid is at least 70%, at least 75%, at least 80%, at least 85%, at least 90%, at least 95%, at least 96%, at least 97%, at least 98%, at least 99%, at least 100% identical to a wildtype AAVrh74 capsid protein. In some embodiments, the AAVrh74 variant capsid comprises a substitution at amino acid position N502. In some embodiments, the substitution at amino acid position N502 is an isoleucine (I). In some embodiments, the AAVrh74 variant capsid protein comprises a substitution at amino acid position W505. In some embodiments, the substitution at amino acid position W505 is an arginine (R).

Different AAV serotypes are used to optimize transduction of particular target cells or to target specific cell types within a particular target tissue (e.g., a diseased tissue, e.g., muscle tissue). A rAAV particle can comprise viral proteins and viral nucleic acids of the same serotype or a mixed serotype. For example, in some embodiments a rAAV particle can comprise AAVrh74 capsid proteins or variants thereof and at least one ITR of a different AAV serotype. In some embodiments, the rAAV particle can comprise AAVrh74 capsid proteins or variants thereof and at least one AAV2 ITR. In some embodiments, the rAAV particle comprises AAVrh74N502I capsid proteins and at least one AAV2 ITR. In some embodiments, the rAAV particle comprises AAVrhW505R capsid proteins and at least one AAV2 ITR.

In some aspects, the invention provides viral particles comprising a recombinant self-complementing genome. rAAV particles with self-complementing genomes and methods of use of self-complementing AAV genomes are described in U.S. Pat. Nos. 6,596,535; 7,125,717; 7,465,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a transgene). In some embodiments, the invention provides a rAAV particle comprising an AAV genome, wherein the rAAV genome comprises a first heterologous polynucleotide sequence (e.g., an RNAi of the present disclosure) and a second heterologous polynucleotide sequence (e.g., antisense strand of an RNAi of the present disclosure) wherein the first heterologous polynucleotide sequence can form intrastrand base pairs with the second polynucleotide sequence along most or all of its length. In some embodiments, the first heterologous polynucleotide sequence and a second heterologous polynucleotide sequence are linked by a sequence that facilitates intrastrand basepairing; e.g., a hairpin DNA structure. Hairpin structures are known in the art, for example in miRNA or siRNA molecules. In some embodiments, the first heterologous polynucleotide sequence and a second heterologous polynucleotide sequence are linked by a mutated ITR (e.g., the right ITR). In some embodiments, the ITR comprises the polynucleotide sequence 5′-CCACTCCCTCTCTGCGCGCTCGCTCGCTCACTGAGGCCGGGCGACCAAAGGTCGC CCGACGCCCGGGCTTTGCCCGGGCGGCCTCAGTGAGCGAGCGAGCGCGCAGAGA GGGA-3 (SEQ ID NO:27). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR. In some embodiments, the invention provides AAV viral particles comprising a recombinant viral genome comprising a functional AAV2 ITR, a first polynucleotide sequence encoding an RNAi of the present disclosure, a mutated AAV2 ITR comprising a deletion of the D region and lacking a functional terminal resolution sequence, a second polynucleotide sequence comprising the complementary sequence to the sequence encoding an RNAi of the present disclosure, of the first polynucleotide sequence and a functional AAV2 ITR.

rAAV particles can be produced using methods known in the art. See, e.g., U.S. Pat. Nos. 6,566,118; 6,989,264; and 6,995,006. In practicing the invention, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

Methods known in the art for production of rAAV vectors include but are not limited to transfection, stable cell line production, and infectious hybrid virus production systems which include adenovirus-AAV hybrids, herpesvirus-AAV hybrids (Conway, J E et al., (1997) J. Virology 71 (11): 8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a nucleic acid (such as a therapeutic nucleic acid) flanked by at least one AAV ITR sequences; and 5) suitable media and media components to support rAAV production. In some embodiments, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors. In some embodiments, the AAV helper functions are provided by adenovirus or HSV. In some embodiments, the AAV helper functions are provided by baculovirus and the host cell is an insect cell (e.g., Spodoptera frugiperda (Sf9) cells).

In some embodiments, rAAV particles may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified. As such, in some embodiments, the rAAV particle was produced by triple transfection of a nucleic acid encoding the rAAV vector, a nucleic acid encoding AAV rep and cap, and a nucleic acid encoding AAV helper virus functions into a host cell, wherein the transfection of the nucleic acids to the host cells generates a host cell capable of producing rAAV particles.

In some embodiments. rAAV particles may be produced by a producer cell line method, such as the exemplary producer cell line method provided infra (see also (referenced in Martin et al., (2013) Human Gene Therapy Methods 24:253-269). Briefly, a cell line (e.g., a HeLa cell line) may be stably transfected with a plasmid containing a rep gene, a capsid gene, and a promoter-heterologous nucleic acid sequence. Cell lines may be screened to select a lead clone for rAAV production, which may then be expanded to a production bioreactor and infected with an adenovirus (e.g., a wild-type adenovirus) as helper to initiate rAAV production. Virus may subsequently be harvested, adenovirus may be inactivated (e.g., by heat) and/or removed, and the rAAV particles may be purified. As such, in some embodiments, the rAAV particle was produced by a producer cell line comprising one or more of nucleic acid encoding the rAAV vector, a nucleic acid encoding AAV rep and cap, and a nucleic acid encoding AAV helper virus functions.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein: (ii) an rAAV pro-vector comprising a nucleic acid encoding an RNAi of the present disclosure as described herein flanked by at least one AAV ITR, and (iii) an AAV helper function; and (b) recovering the rAAV particles produced by the host cell. In some embodiments, the RNAi comprises the nucleotide sequence of SEQ ID NO:7. In some embodiments, said at least one AAV ITR is selected from the group consisting of AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAVrh74, AAVrh74 N502I, AAVrh74 W505R, AAV2R471A, AAV DJ, a goat AAV, bovine AAV, or mouse AAV capsid serotype ITRs or the like. In some embodiments, said encapsidation protein is selected from the group consisting AAVrh74, or a variant thereof, such as AAVrh74 N502I or AAVrh74 W505R. In some embodiments, the encapsidation protein is an AAVrh74 protein. In some embodiments, the encapsidation protein is a variant of an AAVrh74 protein. In some embodiments, the encapsidation protein is an AAVrh74N502I protein. In some embodiments, the encapsidation protein is an AAVrh74W505R protein. In some embodiments, the variant AAVrh74 capsid protein maintains the ability to form an AAV capsid. In some embodiments, the rAAV particles comprise an AAVrh74 N502I capsid and a recombinant genome comprising AAV2 ITRs and nucleic acid encoding an RNAi of the present disclosure. In some embodiments, the rAAV particles comprise an AAVrh74 W505R capsid and a recombinant genome comprising AAV2 ITRs and nucleic acid encoding an RNAi of the present disclosure. In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

Also provided herein are pharmaceutical compositions comprising a rAAV particle comprising a transgene encoding an RNAi of the present disclosure and a pharmaceutically acceptable carrier. The pharmaceutical compositions may be suitable for any mode of administration described herein. A pharmaceutical composition of a rAAV particle comprising a nucleic acid encoding an RNAi of the present disclosure can be introduced systemically. For example, a recombinant viral particle comprising a nucleic acid encoding an RNAi of the present disclosure can be administered intravenously, intra-arterially, subcutaneously or interperitoneally.

In some embodiments, the pharmaceutical compositions comprising a recombinant viral particle comprising a transgene encoding an RNAi of the present disclosure described herein and a pharmaceutically acceptable carrier is suitable for administration to human. Such carriers are well known in the art (see, e.g., Remington's Pharmaceutical Sciences, 15th Edition, pp. 1035-1038 and 1570-1580). In some embodiments, the pharmaceutical compositions comprising a rAAV described herein and a pharmaceutically acceptable carrier is suitable for systemic injection into a mammal.

Such pharmaceutically acceptable carriers can be sterile liquids, such as water and oil, including those of petroleum, animal, vegetable or synthetic origin, such as peanut oil, soy bean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

### Articles of Manufacture and Kits

Also provided are kits or articles of manufacture for use in the methods described herein. In aspects, the kits comprise the compositions described herein (e.g., a rAAV particle of the present disclosure comprising nucleic acid encoding an RNAi of the present disclosure) in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present invention also provides kits comprising compositions described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein. For example, in some embodiments, the kit comprises a composition of recombinant viral particles comprising a transgene encoding an RNAi of the present disclosure for delivery of an effective amount of the rAAV particles to a mammal, a pharmaceutically acceptable carrier suitable for injection into the mammal, and one or more of: a buffer, a diluent, a filter, a needle, a syringe, and a package insert with instructions for performing injections into the mammal. In some embodiments, the kit comprising instructions for treating DM-1 with the rAAV particles described herein. In some embodiments, the kit comprising instructions for using the rAAV particles described herein according to any one of the methods described herein.

### Exemplary Embodiments

The invention includes the following enumerated exemplary embodiments.

1. A recombinant adeno-associated virus (rAAV) particle comprising:
a) an RNAi comprising a first strand and a second strand, wherein:
   i) the first strand and the second strand form a duplex;
   ii) the first strand comprises a guide region, wherein the guide region comprises nucleic acid with the sequence 5′-AGUCGAAGACAGUUCUAGGGU-3′ (SEQ ID NO: 1) or with a sequence with about 90% identity to the sequence of SEQ ID NO: 1; and
   iii) the second strand comprises a non-guide region; and
b) an AAV capsid comprising an amino acid sequence with about 90% identity to a wildtype AAVrh74 capsid.

2. The rAAV particle of embodiment 1, wherein the non-guide region comprises nucleic acid with the sequence 5′ ACCCUAGAUGUCUUCGAUU-3′ (SEQ ID NO:2) or a with a sequence with about 90% identity to the sequence of SEQ ID NO:2.

3. The rAAV particle of embodiment 1 or 2, wherein the first strand comprises nucleic acid with the sequence of SEQ ID NO: 1 and the non-guide region comprises nucleic acid with the sequence of SEQ ID NO:2.

4. The rAAV particle of any one of embodiments 1-3, wherein the first strand and the second strand are linked by means of an RNA linker capable of forming a loop structure.

5. The rAAV particle of embodiment 4, wherein the RNA linker comprises from about 4 to about 50 nucleotides.

6. The rAAV particle of embodiment 4 or 5, wherein the loop structure comprises from about 4 to about 20 nucleotides.

7. The rAAV particle of any one of embodiments 4-6, wherein the loop structure comprises nucleic sequence with of SEQ ID NO:3 or with a sequence with about 90% identity to the sequence of SEQ ID NO:3.

8. The rAAV particle of any one of embodiments 4-7, wherein the RNAi comprises 5′ to 3′ the second strand, the RNA linker, and the first strand.

9. The rAAV particle of any one of embodiments 4-7, wherein the RNAi comprises 5′ to 3′ the first strand, the RNA linker, and the second strand.

10. The rAAV particle of any one of embodiments 1-8, wherein the RNAi comprises nucleic acid with the sequence of SEQ ID NO:7 or with a sequence with about 90% identity to the sequence of SEQ ID NO:7.

11. The rAAV particle of any one of embodiments 1-10, wherein the RNAi is a small inhibitory RNA (siRNA), a microRNA (miRNA), or a small hairpin RNA (shRNA).

12. The rAAV particle of any one of embodiment 1-11, wherein the RNAi further comprises a scaffold.

13. The rAAV particle of embodiment 12, wherein the scaffold comprises all or a portion of the nucleic acid of SEQ ID No: 11.

14. The rAAV particle of embodiment 13, wherein the miRNA is embedded within the scaffold.

15. The rAAV particle of embodiments 14, wherein the scaffold has a 5′ arm, wherein the 5′ arm is located 5′ to the nucleic acid encoding the RNAi, and a 3′ arm, wherein the 3′ arm is located 3′ to the nucleic acid encoding the RNAi.

16. The rAAV particle of any one of embodiments 12-15, wherein the scaffold is a miR-155 scaffold.

17. The rAAV particle of any one of embodiments 12-16, wherein the miR-155 scaffold comprises the nucleic acid of SEQ ID NO:9 or a sequence with about 90% identity to the sequence of SEQ ID NO:9 located 5′ to the RNAi.

18. The rAAV particle of any one of embodiments 12-17, wherein the miR-155 scaffold comprises the nucleic acid of SEQ ID NO: 10 or a sequence with about 90% identity to the sequence of SEQ ID NO: 10 located 3′ to the RNAi.

19. The rAAV particle of any one of embodiments 1-18, wherein the RNAi targets RNA encoding a polypeptide associated with myotonic dystrophy-1 (DM1).

20. The rAAV particle of embodiment 19, wherein the polypeptide is dystrophia myotonica protein kinase (DMPK).

21. The rAAV particle of embodiment 20, wherein the DMPK comprises a mutation associated with DM 1.

22. The rAAV particle of embodiment 20 or 21, wherein the gene encoding DMPK comprises five or more CTG trinucleotide repeats.

23. An expression cassette comprising a nucleic acid sequence encoding the RNAi of any one of embodiments 1-22.

24. The expression cassette of embodiment 23, wherein the nucleic acid encoding the RNAi is operably linked to a promoter.

25. The expression cassette of embodiment 24, wherein the promoter is a muscle-specific promotor.

26. The expression cassette of embodiment 24 or 25, wherein the promoter is a desmin promoter or variant thereof.

27. The expression cassette of embodiment 26, wherein the desmin promoter comprises one or more enhancer elements and the promoter for the human desmin gene.

28. The expression cassette of embodiment 26 or 27, wherein the desmin promoter comprises two enhancer elements and the promoter for the human desmin gene.

29. The expression cassette of any one of embodiments 26-28, wherein the desmin promoter comprises one or more Byrne enhancer elements and/or one or more Paulin enhancer elements.

30. The expression cassette of any one of embodiments 26-29, wherein the desmin promoter comprises one or more enhancer elements comprising the nucleotide sequence of SEQ ID NO:21 or a nucleotide sequence with about 90% identity to the sequence of SEQ ID NO: 21 and/or one or more enhancer elements comprising the nucleotide sequence of SEQ ID NO: 22 or a nucleotide sequence with about 90% identity to the sequence of SEQ ID NO:22.

31. The expression cassette of any one of embodiments 26-30, wherein the desmin promoter comprises the nucleotide sequence of SEQ ID NO: 12 or a sequence with about 90% identity to the nucleotide sequence of SEQ ID NO: 12.

32. The expression cassette of any one of embodiments 23-31, wherein the expression cassette further comprises an intron.

33. The expression cassette of embodiment 32, wherein the intron is a rabbit β-globin intron.

34. The expression cassette of embodiment 32 or 33, wherein the intron comprises the nucleotide sequence of SEQ ID NO:13 or a sequence with about 90% identity to the sequence of SEQ ID NO:13.

35. The expression cassette of any one of embodiments 32-34, wherein the nucleic acid encoding the RNAi is embedded in the intron.

36. The expression cassette of embodiment 35, wherein the intron comprises a 5′ arm and a 3′ arm, wherein the 5′ arm is located 5′ to the nucleic acid encoding the RNAi and the 3′ arm is located 3′ to the nucleic acid encoding the RNAi.

37. The expression cassette of embodiment 36, wherein the 5′ arm of the intron comprises the nucleotide sequence of SEQ ID NO: 14 or a sequence with about 90% identity to the sequence of SEQ ID NO: 14.

38. The expression cassette of embodiment 36 or 37, wherein the 3′ arm of the intron comprises the nucleotide sequence of SEQ ID NO: 15 or a sequence with about 90% identity to the sequence of SEQ ID NO:15.

39. The expression cassette of any one of embodiments 23-38, wherein the expression cassette further comprises a polyadenylation signal.

40. The expression cassette of embodiment 39 wherein the polyadenylation signal is a bovine growth hormone polyadenylation signal, an SV40 polyadenylation signal, or a HSV TK pA.

41. The expression cassette of embodiment 40, wherein the polyadenylation signal is a minimal bovine growth hormone polyadenylation signal.

42. The expression cassette of any one of embodiments 39-41, wherein the bovine growth hormone polyadenylation signal comprises the nucleotide sequence of SEQ ID NO:16 or a sequence with about 90% identity to the sequence of SEQ ID NO:16.

43. The expression cassette of any one of embodiments 23-42, wherein the expression cassette comprises the nucleotide sequence of SEQ ID NO:17 or a sequence with about 90% identity to the sequence of SEQ ID NO: 17.

44. An expression cassette, wherein the expression cassette comprises a modified desmin promoter, wherein the modified desmin promoter comprises one or more enhancer elements and the promoter for the human desmin gene.

45. The expression cassette of embodiment 44, wherein the modified desmin promoter comprises two enhancer elements and the promoter for the human desmin gene.

46. The expression cassette of embodiment 44 or 45, wherein the modified desmin promoter comprises one or more Byrne enhancer elements and/or one or more Paulin enhancer elements.

47. The expression cassette of any one of embodiments 44-46, wherein the modified desmin promoter comprises one or more enhancer elements comprising the nucleotide sequence of SEQ ID NO:21 or a nucleotide sequence with about 90% identity to the sequence of SEQ ID NO:21 and/or one or more enhancer elements comprising the nucleotide sequence of SEQ ID NO:22 or a nucleotide sequence with about 90% identity to the sequence of SEQ ID NO:22.

48. The expression cassette of any one of embodiments 44-47, wherein the desmin promoter comprises the nucleotide sequence of SEQ ID NO: 12 or a sequence with about 90% identity to the nucleotide sequence of SEQ ID NO: 12.

49. The expression cassette of any one of embodiments 44-48, wherein the expression cassette further comprises an intron.

50. The expression cassette of embodiment 49, wherein the intron is a rabbit β-globin intron.

51. The expression cassette of embodiment 49 or 50, wherein the intron comprises the nucleotide sequence of SEQ ID NO: 13 or a sequence with about 90% identity to the sequence of SEQ ID NO: 13.

52. The expression cassette of any one of embodiments 44-51, wherein the nucleic acid encoding the transgene is embedded in the intron.

53. The expression cassette of embodiment 52, wherein the intron comprises a 5′ arm and a 3′ arm, wherein the 5′ arm is located 5′ to the nucleic acid encoding the transgene and the 3′ arm is located 3′ to the nucleic acid encoding the transgene.

54. The expression cassette of embodiment 53, wherein the 5′ arm of the intron comprises the nucleotide sequence of SEQ ID NO: 14 or a sequence with about 90% identity to the sequence of SEQ ID NO: 14.

55. The expression cassette of embodiment 53 or 54, wherein the 3′ arm of the intron comprises the nucleotide sequence of SEQ ID NO:15 or a sequence with about 90% identity to the sequence of SEQ ID NO:15.

56. The expression cassette of any one of embodiments 44-55, wherein the expression cassette further comprises a polyadenylation signal.

57. The expression cassette of embodiment 56, wherein the polyadenylation signal is a bovine growth hormone polyadenylation signal, an SV40 polyadenylation signal, or a HSV TK pA.

58. The expression cassette of embodiment 57, wherein the polyadenylation signal is a minimal bovine growth hormone polyadenylation signal.

59. The expression cassette of any one of embodiments 56-58, wherein the bovine growth hormone polyadenylation signal comprises the nucleotide sequence of SEQ ID NO:16 or a sequence with about 90% identity to the sequence of SEQ ID NO: 16.

60. The expression cassette of any one of embodiments 44-59, wherein the transgene encodes a polypeptide or a nucleic acid.

61. The expression cassette of any one of embodiments 44-60, wherein the transgene encodes an RNAi.

62. A vector comprising the expression cassette of any one of embodiments 23-61.

63. The vector of embodiment 62, wherein the expression cassette is flanked by one or more stuffer nucleic acid sequences.

64. The vector of embodiment 63, wherein the one or more stuffer nucleic acid sequences is derived from the human SerpinA1 gene.

65. The vector of embodiment 63 or 64, wherein a stuffer nucleic acid sequence located 5′ to the expression cassette is derived from the human SerpinA1 gene.

66. The vector of any one of embodiments 63-65, wherein a stuffer sequence located 5′ to the expression cassette comprises the nucleotide sequence of SEQ ID NO: 18 or a sequence with about 90% identity to the sequence of SEQ ID NO: 18.

67. The vector of any one of embodiments 63-66, wherein a stuffer nucleic acid sequence located 3′ to the expression cassette is derived from the human SerpinA1 gene.

68. The vector of any one of embodiments 63-67, wherein a stuffer sequence located 3′ to the expression cassette comprises the nucleotide sequence of SEQ ID NO: 19 or a sequence with about 90% identity to the sequence of SEQ ID NO: 19.

69. The vector of any one of embodiments 62-68, wherein the vector is a recombinant adeno-associated virus (rAAV) vector.

70. The rAAV vector of embodiment 69, wherein the expression cassette is flanked by one or more AAV inverted terminal repeat (ITR) sequences.

71. The rAAV vector of embodiment 70, wherein the expression cassette is flanked by two AAV ITRs.

72. The rAAV vector of embodiment 70 or 71, wherein the AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype ITRs.

73. The rAAV vector of any one of embodiments 70-72, wherein the AAV ITRs are AAV2 ITRs.

74. The rAAV vector of any one of embodiments 69-73, wherein the rAAV vector comprises the nucleotide sequence of SEQ ID NO:20 or a sequence with about 90% identity to the sequence of SEQ ID NO:20.

75. The rAAV vector of any one of embodiments 69-74, wherein the vector is a self-complementary rAAV vector.

76. A cell comprising the expression cassette of any one of embodiments 23-61, the vector of any one of embodiments 62-68, or the rAAV vector of any one of embodiments 69-75.

77. The rAAV particle of any one of embodiments 1-22, wherein the AAV capsid comprises an amino acid sequence comprising an amino acid substitution at position 502.

78. The rAAV particle of embodiment 77, wherein the amino acid substitution at position 502 is an isoleucine (I).

79. The rAAV particle of embodiment 77 or 78, wherein the rAAV particle comprises a AAVrh74 N502I serotype capsid.

80. The rAAV particle of embodiment 77 or 78, wherein the ITR is an AAV2 ITR and the capsid of the rAAV particle is an AAVrh74 N502I serotype capsid

81. The rAAV particle of any one of embodiments 1-22, wherein the AAV capsid comprises an amino acid sequence comprising an amino acid substitution at position 505.

82. The rAAV particle of embodiment 81, wherein the amino acid substitution at position 505 is an arginine (R).

83. The rAAV particle of any one of embodiments 1-22, wherein the rAAV particle comprises a AAVrh74 W505R serotype capsid.

84. The rAAV particle of embodiment 83, wherein the ITR is an AAV2 ITR and the capsid of the rAAV particle is an AAVrh74 W505R serotype capsid.

85. An rAAV particle comprising an rAAV vector and a capsid, wherein the rAAV vector comprises the following nucleic acids 5′ to 3′, an AAV2 ITR, nucleic acid encoding a stuffer nucleic acid sequence from the human serpinA1 gene, a Bymne desmin enhancer element, a Paulin desmin enhancer element, a desmin promoter, a 5′ arm of a rabbit β-globin intron, a 5′ miR155 scaffold sequence, a DMPK²⁰⁴ miRNA guide sequence, a miR155 terminal loop sequence, a DMPK²⁰⁴ miRNA passenger sequence, a 3′ miR155 scaffold sequence, a 3′ arm of a rabbit β-globin intron, a minimal bovine growth hormone polyadenylation sequence, nucleic acid encoding a stuffer nucleic acid sequence from the human serpinA1 gene, and an AAV2 ITR; and wherein the capsid is an AAVrh74 N502I capsid.

86. An rAAV particle comprising an rAAV vector, wherein the rAAV vector comprises the following nucleic acids 5′ to 3′, an AAV2 ITR comprising the polynucleotide sequence of SEQ ID NO:43, nucleic acid encoding a stuffer nucleic acid sequence from the human serpinA1 gene comprising the polynucleotide sequence of SEQ ID NO:18, a Byrne desmin enhancer element comprising the polynucleotide sequence of SEQ ID NO:21, a Paulin desmin enhancer element comprising the polynucleotide sequence of SEQ ID NO:22, a desmin promoter comprising the polynucleotide sequence of SEQ ID NO:23, a 5′ arm of a rabbit β-globin intron comprising the polynucleotide sequence of SEQ ID NO: 14, a 5′ miR155 scaffold sequence comprising the polynucleotide sequence of SEQ ID NO:40, a DMPK²⁰⁴ miRNA guide sequence comprising the polynucleotide sequence of SEQ ID NO:4, a miR 155 terminal loop sequence comprising the polynucleotide sequence of SEQ ID NO:6, a DMPK²⁰⁴ miRNA passenger sequence comprising the polynucleotide sequence of SEQ ID NO: 5, a 3′ miR 155 scaffold sequence comprising the polynucleotide sequence of SEQ ID NO: 41, a 3′ arm of a rabbit β-globin intron comprising the polynucleotide sequence of SEQ ID NO: 15, a minimal bovine growth hormone polyadenylation sequence comprising the polynucleotide sequence of SEQ ID NO: 16, nucleic acid encoding a stuffer nucleic acid sequence from the human serpinA1 gene comprising the polynucleotide sequence of SEQ ID NO: 19, and an AAV2 ITR comprising the polynucleotide sequence of SEQ ID NO:49; and wherein the capsid is an AAVrh74 N502I capsid.

87. The rAAV particle of embodiment 85 or 86, wherein the AAVrh74 N502I capsid comprises capsid proteins comprising the amino acid sequence of SEQ ID NO:50.

88. An rAAV particle comprising an rAAV vector and a capsid, wherein the rAAV vector comprises the following nucleic acids 5′ to 3′, an AAV2 ITR, nucleic acid encoding a stuffer nucleic acid sequence from the human serpinA1 gene, a Byme desmin enhancer element, a Paulin desmin enhancer element, a desmin promoter, a 5′ arm of a rabbit β-globin intron, a 5′ miR155 scaffold sequence, a DMPK²⁰⁴ miRNA guide sequence, a miR155 terminal loop sequence, a DMPK²⁰⁴ miRNA passenger sequence, a 3′ miR155 scaffold sequence, a 3′ arm of a rabbit β-globin intron, a minimal bovine growth hormone polyadenylation sequence, nucleic acid encoding a stuffer nucleic acid sequence from the human serpinA1 gene, and an AAV2 ITR; and wherein the capsid is an AAVrh74 W505R capsid.

89. An rAAV particle comprising an rAAV vector, wherein the rAAV vector comprises the following nucleic acids 5′ to 3′, an AAV2 ITR comprising the polynucleotide sequence of SEQ ID NO:43, nucleic acid encoding a stuffer nucleic acid sequence from the human serpinA1 gene comprising the polynucleotide sequence of SEQ ID NO: 18, a Byme desmin enhancer element comprising the polynucleotide sequence of SEQ ID NO:21, a Paulin desmin enhancer element comprising the polynucleotide sequence of SEQ ID NO:22, a desmin promoter comprising the polynucleotide sequence of SEQ ID NO:23, a 5′ arm of a rabbit β-globin intron comprising the polynucleotide sequence of SEQ ID NO: 14, a 5′ miR155 scaffold sequence comprising the polynucleotide sequence of SEQ ID NO:40, a DMPK²⁰⁴ miRNA guide sequence comprising the polynucleotide sequence of SEQ ID NO:4, a miR 155 terminal loop sequence comprising the polynucleotide sequence of SEQ ID NO:6, a DMPK²⁰⁴ miRNA passenger sequence comprising the polynucleotide sequence of SEQ ID NO: 5, a 3′ miR 155 scaffold sequence comprising the polynucleotide sequence of SEQ ID NO: 41, a 3′ arm of a rabbit β-globin intron comprising the polynucleotide sequence of SEQ ID NO: 15, a minimal bovine growth hormone polyadenylation sequence comprising the polynucleotide sequence of SEQ ID NO: 16, nucleic acid encoding a stuffer nucleic acid sequence from the human serpinA1 gene comprising the polynucleotide sequence of SEQ ID NO: 19, and an AAV2 ITR comprising the polynucleotide sequence of SEQ ID NO:49; and wherein the capsid is an AAVrh74 W505R capsid.

90. The rAAV particle of embodiment 88 or 89, wherein the AAVrh74 W505R capsid comprises capsid proteins comprising the amino acid sequence of SEQ ID NO: 52.

91. A composition comprising the rAAV particle of any one of claims 1-22 and 77-90.

92. A pharmaceutical composition comprising the rAAV particle of any one of claims 1-22 and 77-90.

93. The composition of embodiment 91 or 92, wherein the composition further comprises a pharmaceutically acceptable carrier.

94. A kit comprising the rAAV particle of any one of embodiments 1-22 and 77-90.

95. A kit comprising the rAAV particle of any one of claims 1-22 and 77-90.

96. A kit comprising the composition of any one of embodiments 91-93.

97. The kit of any one of embodiments 94-96, further comprising instructions for use.

98. A method for treating myotonic dystrophy-1 (DM1) in a mammal in need thereof comprising administering to the mammal an effective amount of the rAAV particle of any one of embodiments 1-22 and 77-90.

99. A method for inhibiting the expression of dystrophia myotonica protein kinase (DMPK) in a mammal with DM-1 in need thereof comprising administering to the mammal an effective amount of the rAAV particle of any one of embodiments 1-22 and 77-90.

100. A method for inhibiting the accumulation of DMPK RNA in a cell of a mammal with DM-1 in need thereof comprising administering to the mammal an effective amount of the RNAi of any one of embodiments 1-22 and 77-90.

101. A method for treating myotonic dystrophy-1 (DM1) in a mammal in need thereof comprising administering to the mammal an effective amount of the rAAV particle of any one of claims 1-22 and 77-90.

102. A method for inhibiting the expression of dystrophia myotonica protein kinase (DMPK) in a mammal with DM-1 in need thereof comprising administering to the mammal an effective amount of the rAAV particle of any one of claims 1-22 and 77-90.

103. A method for inhibiting the accumulation of DMPK RNA in a cell of a mammal with DM-1 in need thereof comprising administering to the mammal an effective amount of the rAAV particle of any one of claims 1-22 and 77-90.

104. The method of any one of embodiments 100-104, wherein the effective amount of the viral particle or rAAV particle is a dose of about 1×10⁸ to about 2×10¹³ genome copies/mL.

105. The method of embodiment 104, wherein the dose is about 5×10¹² genome copies/mL.

106 The method of embodiment 104, wherein the dose is about 1×10¹³ genome copies/mL.

107. The method of embodiment 104, wherein the dose is about 2×10¹³ genome copies/mL.

10⁸. The method of any one of embodiments 101-103, wherein the effective amount of the viral particle or rAAV particle is a dose of about 1×10⁸ to about 2×10¹⁴ genome copies/kg of body weight.

10⁹. The method of embodiment 10⁸, wherein the dose is about 5×10¹³ genome copies/kg of body weight.

110. The method of embodiment 10⁸, wherein the dose is about 1×10¹⁴ genome copies/kg of body weight.

111. The method of embodiment 10⁸, wherein the dose is about 2×10¹⁴ genome copies/kg of body weight.

112. A method for treating myotonic dystrophy-1 (DM1) in a mammal in need thereof comprising administering to the mammal an effective amount of the composition of any one of embodiments 91-93.

113. A method for inhibiting the expression of dystrophia myotonica protein kinase (DMPK) in a mammal with DM-1 in need thereof comprising administering to the mammal an effective amount of the composition of any one of embodiments 91-93.

114. A method for inhibiting the accumulation of DMPK RNA in a cell of a mammal with DM-1 in need thereof comprising administering to the mammal an effective amount of the composition of any one of embodiments 91-93.

115. The method of any one of embodiments 98-100, wherein the RNAi is administered in combination with an immunosuppressive agent, wherein the immunosuppressive agent is administered before, at the same time, and/or after administration of the RNAi.

116. The method of any one of embodiments 101-103, wherein the viral particle or the rAAV particle is administered in combination with an immunosuppressive agent, wherein the immunosuppressive agent is administered before, at the same time, and/or after administration of the viral particle or the rAAV particle.

117. The method of any one of embodiments 112-114, wherein the composition is administered in combination with an immunosuppressive agent, wherein the immunosuppressive agent is administered before, at the same time, and/or after administration of the composition.

### EXAMPLES

The presently disclosed subject matter will be better understood by reference to the following Examples, which are provided as exemplary of the invention, and not by way of limitation.

### Example 1: Generation of the amiR-DMPK²⁰⁴ Expression Cassettes

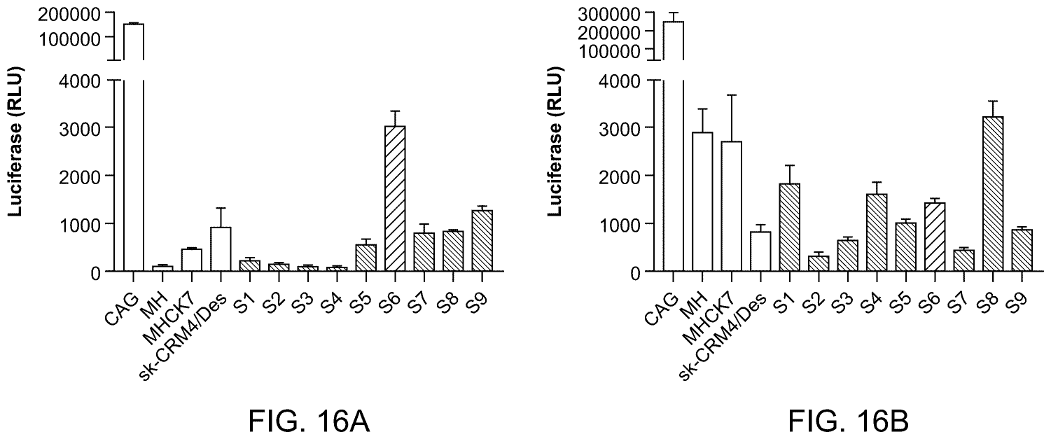

A series of nine transcriptional promoters (S1-S9) were rationally designed with the goal of identifying candidates with high activity in muscle and lower activity in liver. A panel of promoters were screened in primary human myoblasts and Huh7 hepatoma cells to represent muscle and liver, respectively. The newly designed promoters were compared to the ubiquitous CAG promoter and to a panel of previously published promoter sequences with high activity in muscle cells: the synthetic MH promoter which comprises four muscle-specific transcription factor binding sites (TFBSs) and a core promoter fragment of the chicken skeletal muscle α-actin gene, the chimeric MHCK7 promoter, which was designed to target cardiac muscle and combines: the α-MCK enhancer and MCK promoter with the addition of an enhancer from the mouse α-myosin heavy chain; and sk-CRM4/Des, a synthetic promoter combining a skeletal muscle specific cis-regulatory module, the desmin promoter and the MVM intron. For screening, cells were transfected with a plasmid containing a luciferase gene under the control of the promoter of interest. After 48 hours, relative luciferase activity was measured in myoblasts (FIG. 16A) and in Huh7 cells (FIG. 16B). Apart from the CAG promoter, which drives high expression throughout the body, the S6 promoter drove the highest expression in muscle cells, compared to all other newly designed promoters. Expression from the S6 promoter in liver was relatively low. Thus, the S6 promoter was selected for the AAV expression construct.

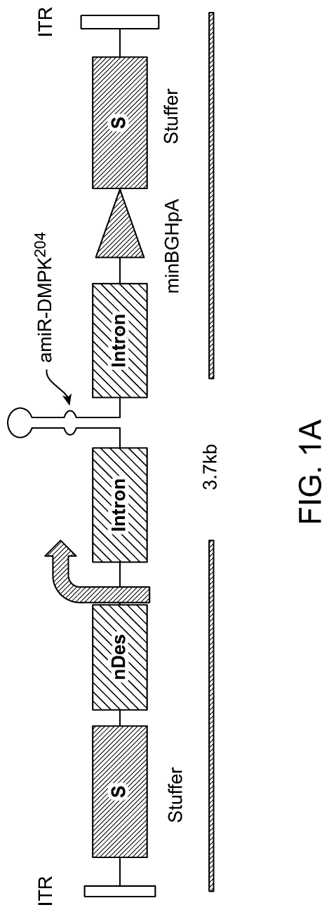

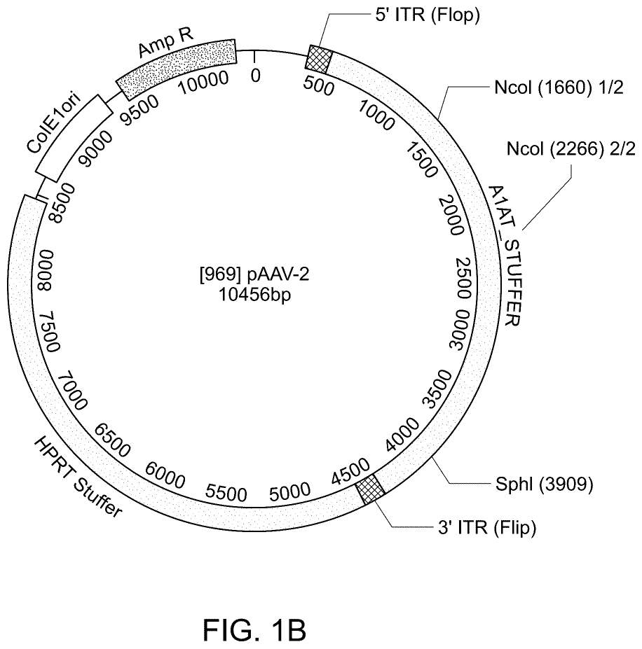

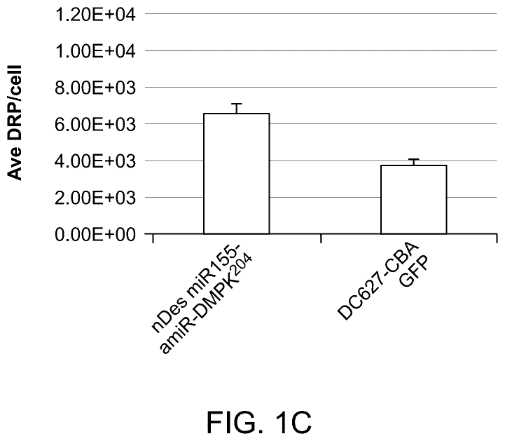

A single-stranded AAV viral vector encoding a microRNA (amiR-DMPK²⁰⁴) designed to target the DMPK gene was generated (FIG. 1A). The construct was designed such that the amiR-DMPK²⁰⁴ microRNA was embedded in an optimized microRNA backbone, miR155 (BLOCK-IT: Thermofisher Catalog nos. K4935-00, K4936-00, K4937-00, K4938-00), and is hereafter “miR155 amiR-DMPK²⁰⁴” or “amiR155-DMPK²⁰⁴”. miR155 amiR-DMPK²⁰⁴ is flanked by rabbit β-globin intron sequences and was placed under the regulation of a hybrid muscle promoter (nDes: the “S6” promoter).

The nDesmin promoter comprising the Byrne desmin enhancer, one copy of the Paulin desmin enhancer (−973 to −693) and the promoter of the human Desmin gene (−228 to +75) were synthesized by conventional oligonucleotide synthesis (Genscript, USA).

A bovine growth hormone polyadenylation sequence was placed 3′ of the intron-flanked amiR-DMPK²⁰⁴ microRNA (minBGHpA). A filler sequence (“stuffer”) was included. The whole gene cassette is flanked by wild-type AAV serotype 2 Inverted Terminal Repeats (ITRs) sequences, for DNA rescue and replication, as well as packaging into an AAV capsid. The sequences were engineered into the ITR plasmid which was used to generate the vector for in vivo efficacy studies.

### 5′ and 3′ ITR Sequences

The ITR sequences were the AAV2 wild-type sequence of 145 bp. The 3′ ITR (downstream of expression cassette) was in the Flip orientation (GenBank: LQ493091.1). The 5′ ITR (upstream of the expression cassette) was in the Flop orientation (145 bp) (Miller et al., 2004, Nature Genetics 36.7 (2004): 767-773). The accuracy of the sequence was confirmed by Sanger sequencing.

nDes Promoter

The nDes promoter was constructed using desmin promoter elements shown in the literature (Li and Paulin, et. al. 1991, J Biol Chem. 266.10:6562-6570). The nDes promoter comprises one copy of the Byrne desmin enhancer, one copy of the Paulin desmin enhancer (−973 to −693) and the promoter of the human desmin gene (−228 to +75).

### 5′ and 3′ Arms of the Rabbit β-Globin Intron

This intron was used to flank the amiR-DMPK²⁰⁴ cassettes because intronic expression of miRNAs is known to enhance target knockdown.

amiR-DMPK²⁰⁴ with the miR155 Scaffold (miR155-amiRDMPK²⁰⁴)

Endogenous miRNAs are hairpin-like secondary structures found in many primary RNA transcripts (pri-miRNAs). In the nucleus, the microprocessor, Drosha/DGCR8 complex binds and cleaves the basal stem of pri-miRNAs to liberate the stem-loop precursor miRNA (pre-miRNA). Pre-miRNAs are then exported from the nucleus where the loop is cleaved by Dicer/TRBP to form a mature RNA duplex. The guide strand, also known as the targeting strand, is separated from the passenger strand and loaded onto an argonaute protein in the RNA induced silencing complex (RISC), which then targets complementary mRNA transcripts for degradation or translational repression.

The amiR-DMPK²⁰⁴ sequence was identified as target for DM1 therapy. The target sequence of amiR-DMPK²⁰⁴ is located upstream of the “CUG” repeat sequences within the 3′ UTR of the DMPK nucleotide sequence. Therefore, amiR-DMPK²⁰⁴ can suppress both wild-type and mutant DMPK transcripts.

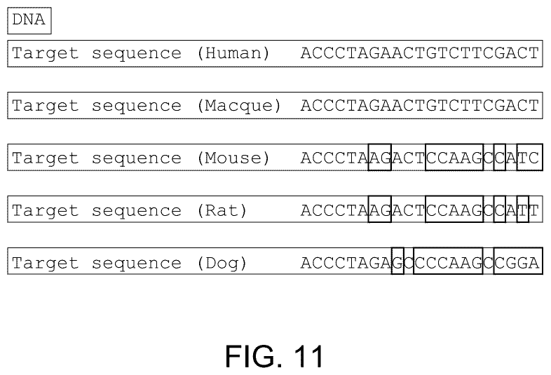

Additionally, the amiR-DMPK²⁰⁴ target region is conserved in non-human primates (NHPs-cynomolgus monkey), and humans, allowing pre-clinical assessment of DMPK knockdown in NHPs (FIG. 11). The amiR-DMPK²⁰⁴ microRNA was evaluated and it was shown that the miR155 scaffold has efficient guide processing with minimal passenger strand processing, reducing the likelihood of off-target effects (see Example 2 below).

The final construct that was selected for development was amiR-DMPK²⁰⁴ with the miR155 scaffold (amiR155-DMPK²⁰⁴), where the guide strand, when processed, targeted DMPK mRNA for degradation. The engineered pre-miRNA sequence structure is based on the murine miR-155 sequence (Lagos-Quintana et al., 2002, Current Biology, 12:9, 735-739). The 5′ and 3′ flanking regions derived from the miR-155 transcript were inserted in the vector to preserve as much as possible of the miR-155 structure. The stem-loop structure was optimized and a 2 nucleotide internal loop resulted in higher knockdown rate than the 5 nucleotide/3 nucleotide internal loop found in native miR-155 molecule (source: BLOCK-iT™ Pol II miR RNAi Expression Vector, (Invitrogen)). The vector, nDes-miR155-amiR-DMPK²⁰⁴, in the context of a AAV capsid, was shown to have potent in vivo activity in the DMSXL mouse model of DM1 (see Example 4 below).

### Minbgh Polya

A minimal BGH poly A site of 186 bps was inserted downstream of the amiR-DMPK²⁰⁴ sequence to allow transcription termination and polyadenylation of the mRNA.

Stuffer Sequence from A1AT Intron

A stuffer sequence from the alpha-1 antitrypsin gene intron sequence 4 was used to bring the gene cassette to the packaging limits for rAAV vectors.

Cloning of the ITR Plasmid with the nDes-miR 155-amiR-DMPK²⁰⁴ Cassette (nDes-miR155-Amir-Dmpk²⁰⁴)

A 1938 bp ITR-ITR sequence of the expression construct, nDes-miR155-amiR-DMPK²⁰⁴-minBGHpolyA cassette, was designed. Cloning of the nDes-miR155-amiR-DMPK²⁰⁴-minBGHpoly A cassette into the ITR plasmid was performed. The synthesized nDes-miR155-amiR-DMPK²⁰⁴-minBGHpoly A included a 5′ NcoI site and 3′ SphI site for cloning into ITR plasmid (FIG. 1B). Briefly, the plasmid containing the synthesized nDes-miR155-amiR-DMPK²⁰⁴-minBGHpolyA sequence was digested with NcoI and SphI and the 1.9 KB fragment was gel purified. The ITR plasmid was digested with NcoI and SphI, dephosphorylated using Calf Intestinal Alkaline Phosphatase (New England Biolabs; Cat No M0290) and the 8.2 KB vector backbone fragment was gel purified. The digested 1.9 KB fragment containing the expression cassette and the digested ITR plasmid were ligated to produce plasmid ITR-nDes-miR 155-amiR-DMPK²⁰⁴.

Small-Scale rAAV Vector Production

A small-scale packaging assay was performed in HEK 293 cells to confirm packaging of the ITR-nDes-miR155-amiR-DMPK²⁰⁴ plasmid. Small-scale production was performed using the AAV rep/cap plasmid. FIG. 1C shows the amount of vector produced per HEK 293 cell as compared to a standard EGFP plasmid gene cassette.

To determine the potential of amiR-DMPK²⁰⁴ to correct the DM1 phenotype, the ability to silence the human DMPK transcript and correct splicing defects in skeletal myoblast cultures from DM1 patients was tested. Due to the CTG expansion in the 3′ UTR of the DMPK gene, DMPK mRNA hairpin structures aggregate as insoluble ribonuclear foci and sequester several RNA-binding proteins. The resulting redistribution of essential splicing factors, such as muscleblind-like 1 (MBNL1), causes mis-splicing of downstream effectors responsible for the differentiation of muscle tissue. The treatment of DM1 patient cells with amiR-DMPK²⁰⁴ caused over 50% DMPK mRNA silencing and splicing correction, as measured by MBNL1 exon 7 inclusion.

### Example 2: Expression of miR155-amiRDMPK²⁰⁴ Corrects DM1 Phenotypes In Vitro

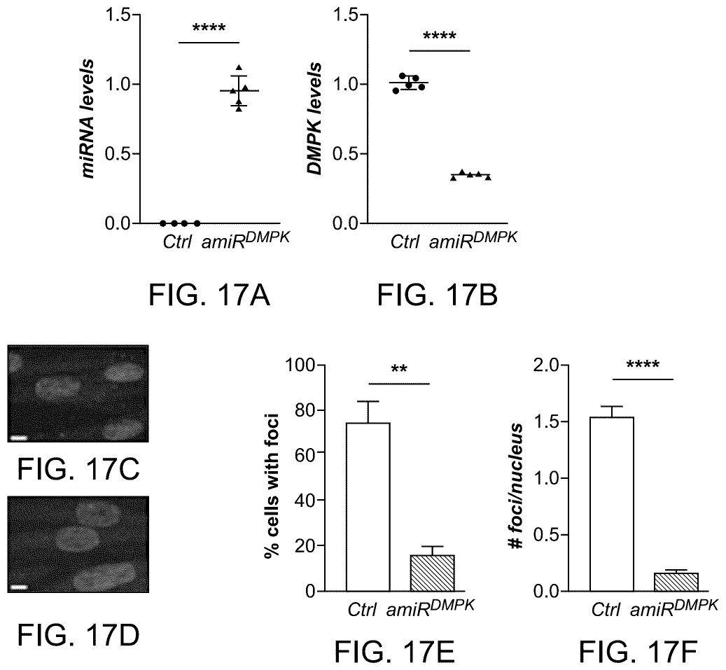

Expression of the miR155-amiRDMPK²⁰⁴ construct driven by the nDes promoter was tested in two in vitro models of DM1. DM1 immortalized human myotubes were transduced with an AAV2 vector containing an artificial miRNA targeting DMPK under the control of the nDes promoter. DMPK amiRNA (FIG. 17A) and DMPK mRNA (FIG. 17B) was measured by PCR. As expected, the DMPK amiRNA was only detectable in treated cells, where DMPK levels were reduced by 65% compared to untreated cells (untreated=1±0.04; treated=0.34±0.018; Mean±SD; p<0.0001, two-tailed unpaired t-test).

One of the hallmarks of DM1 is the presence of nuclear RNA foci. Expansion of the CUG repeat in the 3′-UTR of the DMPK mRNA leads to retention of the mRNA in the nucleus where it binds to and sequesters important RNA binding proteins, including muscleblind-like protein 1 (MBNL1), which regulates pre-mRNA splicing an mRNA localization. In addition, the presence of the mutant D) MPK transcript is thought to lead to the hyperphosphorylation and stabilization of CELF1 (CUG RNA-binding protein and embryonically lethal abnormal vision-type RNA-binding protein 3-like factor 1), which also functions in the regulation of alternative splicing and RNA stability. Using fluorescent in situ hybridization (FISH), the percentage of cells presenting nuclear foci, as well as the number of foci/nuclei in treated and untreated DM1 human myotubes was quantified (FIG. 17C-17D). Treatment with nDes-miR 155-amiR-DMPK²⁰⁴ reduced the percentage of cells containing foci by 60% (untreated=74.36%±9.3; treated=15.36%±4.2. MEAN±SEM; p=0.0044, two-tailed unpaired t-test) (FIG. 17E). In addition, when compared to untreated cells, treated cells had significantly fewer foci per cell (untreated=1.55±0.08; treated=0.15=0.03. MEAN±SEM; p<0.0001, two-tailed unpaired t-test) (FIG. 17F).

### Example 3: Processing of miR155 amiR-DMPK²⁰⁴

The amiR-DMPK²⁰⁴ was packaged into an AAV capsid and DMPK knockdown efficacy, passenger strand activity, and processing patterns were analyzed in vivo. The constructs harboring nDes-miR 155-amiR-DMPK²⁰⁴ were packaged into AAV. The vectors were intravenously injected into the DMSXL adult humanized DM1 mice model, which expresses human DMPK with >1.000 CTG repeats. After eight weeks, animals were euthanized, multiple tissues were collected to measure DMPK knockdown efficacy, and heart tissue was selected to measure the passenger strand activity and processing patterns.

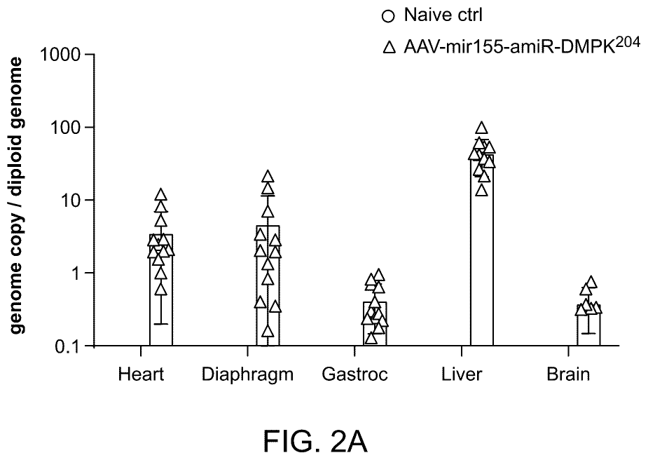

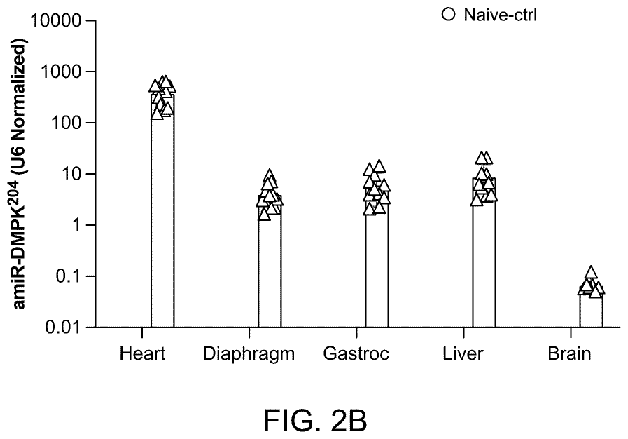

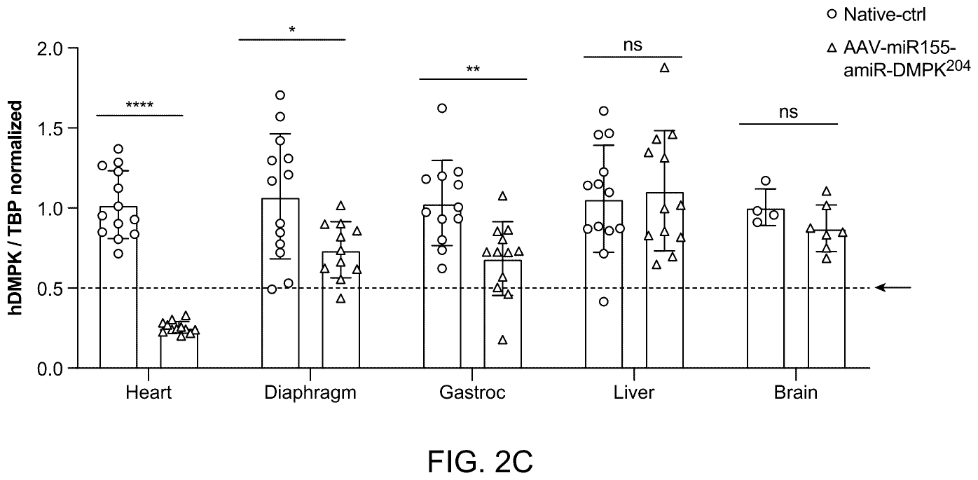

RT-PCR analysis showed robust expression of amiR-DMPK²⁰⁴ in multiple muscle tissues with higher expression in the heart (FIG. 2B). Concomitant with amiR-DMPK²⁰⁴ expression, RT PCR analysis confirmed robust DMPK suppression in the heart with an average of >70% and ˜30% DMPK suppression in different skeletal muscles (FIG. 2C). In heart tissue, DMPK expression is below 50% relative to TBP (TATA binding protein) expression. Notably, DSMXL mice expressing low levels of DMPK relative to TBP (˜50%, indicated by the dotted line in FIG. 2C) do not have an obvious DM1 phenotype. Interestingly, the nDesmin promoter showed strong activity in the cardiac tissue and similar levels in skeletal muscle (FIG. 2B) even though higher transduction was observed in the liver (FIG. 2A). This suggests expression was mainly restricted to the cardiac and skeletal muscle, affected by DM1 pathology.

To assess the processing of the amiR-DMPK²⁰⁴, heart tissues were analyzed for the mature amiR-DMPK²⁰⁴ lengths and sequence composition of the guide and passenger strands by NGS for small transcriptome analysis.

The processing of the amiR-DMPK²⁰⁴ did not produce passenger strands, amiR-DMPK²⁰⁴ was processed exclusively into guide strands (>99%) in mouse cardiomyocytes, but often produced longer stands than the predicted from the miRBase database (Tables 1), miR155 processing most often generated mature lengths between 22 and 26 nt long but processed accurately at 5′ end Table 1. The sequence distributions of the different guide strand lengths (nt) mapping to miR155 amiR-DMPK²⁰⁴ calculated as percentages (% reads). The expected amiR-DMPK²⁰⁴ guide strand is underlined and seed sequence is in Bold. The asterisk indicates reads corresponding to the predicted length of the amiR-DMPK²⁰⁴ guide strand.

TABLE 1
The sequence distributions of the different
guide strand lengths (nt) mapping to miR155
amiR-DMPK²⁰⁴ calculated as percentages (%
reads). The expected amiR-DMPK²⁰⁴ guide strand
is underlined. The asterisk indicates reads
corresponding to the predicted length of the
amiR-DMPK204 guide strand.

| | | % | SEQ ID |
|---|---|---|---|
| miR-155 read sequence | Length | Seqs | NO |
| AGTCGAAGACAGTTCTAGGGTGT | 23 | 52 | 33 |
| AGTCGAAGACAGTTCTAGGGTGTT | 24 | 24 | 34 |
| AGTCGAAGACAGTTCTAGGGTGTTT | 25 | 11 | 35 |
| AGTCGAAGACAGTTCTAGGGT* | 22 | 6 | 36 |
| AGTCGAAGACAGTTCTAGGGTGTTTT | 26 | 3 | 37 |
| AGTCGAAGACAGTTCTAGGGTG | 22 | 4 | 38 |

Overall, no passenger strands were detected by the amiR-DMPK²⁰⁴ with miR155 (The % guide is >99%). Therefore, miR155 was selected as the lead for the pre-clinical studies, as the miR 155 miRNA scaffold is well-validated for RNAi.

### Example 4: Dose Dependent Suppression of Human DMPK by Systemic Injection of AAV Encoding miR155-amiRDMPK²⁰⁴ in Transgenic Mice

To determine the most efficacious dose, the delivery of three separate doses of myotropic AAV (WO/2019/207132) capsid was investigated. AAV encoding the expression cassette for amiR-DMPK²⁰⁴ with the miR155 scaffold (amiR155-DMPK²⁰⁴) was evaluated in a dose-escalation study. Eight-week-old DMSXL mice, which carry a human DMPK transgene containing a CTG expansion of more than 1,000 repeats, were injected intravenously with 5.0×10¹¹ vector genomes (vg)/kg, 5×10¹² vg/kg and 1.0×10¹³ vg/kg, corresponding to low, intermediate, and high doses. Mice were analyzed for clinical symptoms such as body weight, survival, myotonia and cardiac function at 8 weeks following AAV infusion. Mice were euthanized 8 weeks post gene transfer, and DMPK suppression and splicing correction were measured. miR155-amiRDMPK²⁰⁴ expression levels were measured by small RNA TaqMan, and mRNA input levels was normalized to u6 small nuclear RNA.

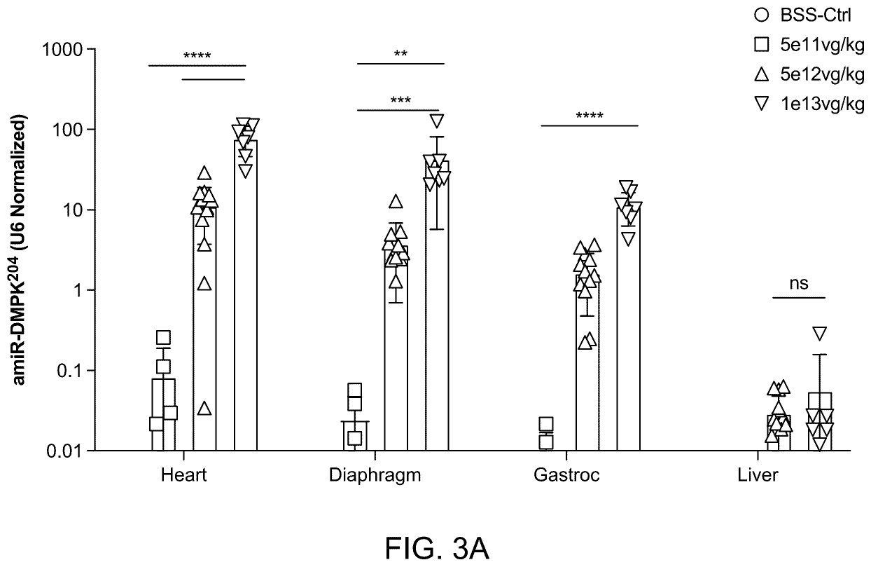

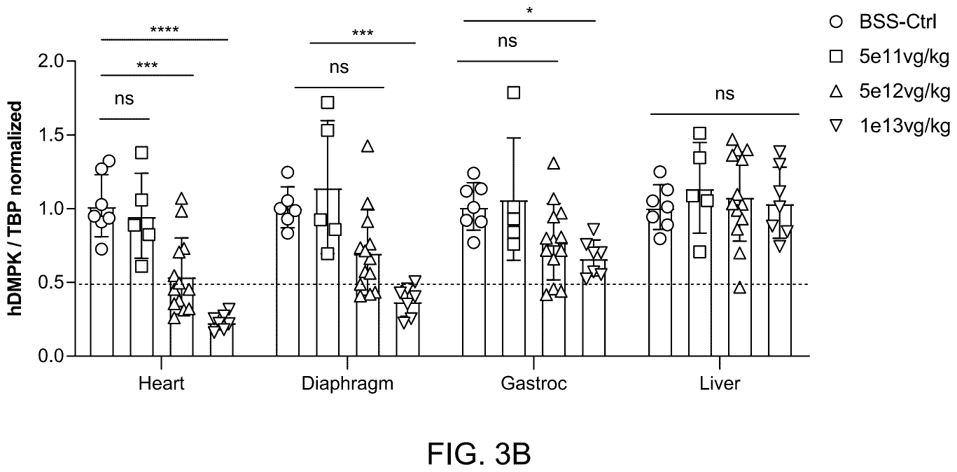

The expression of amiR155-DMPK²⁰⁴ was observed in a dose-dependent manner (FIG. 3A) and resulted in a dose-dependent reduction of total DMPK expression (FIG. 3B) in multiple tissues. Overall, it was observed that ˜10 amiR155-DMPK²⁰⁴ copies/U6 was sufficient to reduce DMPK by >50% in the heart and diaphragm, an amount which may be sufficient to treat DM1 patients.

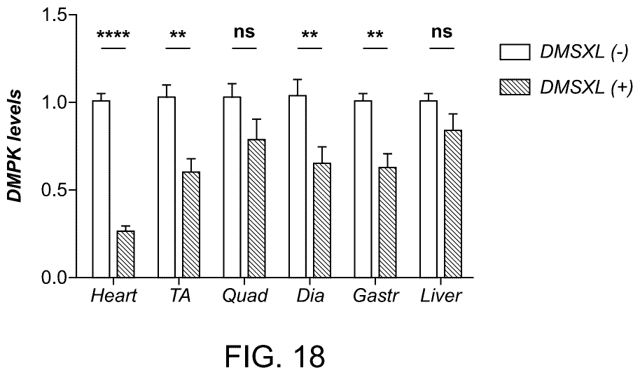

A cohort of DMSXL mice were treated with a single dose of miR155-DMPK²⁰⁴ at a dose of 9×10¹³ vg/kg, which resulted in expression of the artificial miRNA in skeletal and cardiac muscles and significant silencing of DMPK mRNA in the heart, TA, diaphragm and gastrocnemius, but not in liver, confirming the muscle-specificity of the platform at a higher dose (FIG. 18).

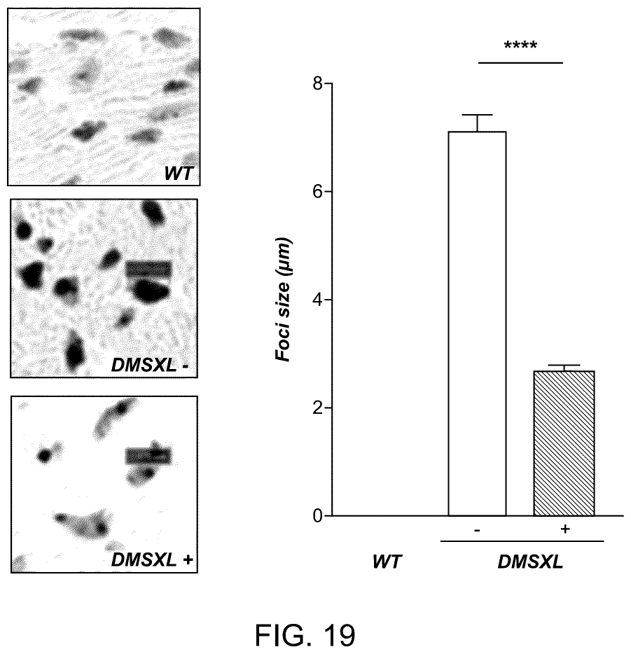

Nuclear RNA foci in the hearts of treated mice were measured by in situ hybridization. The foci were often present as large formations, and therefore the linear size of each formation in positive cells was measured. Treatment with miR155-amiRDMPK²⁰⁴ significantly reduced the size of the foci (untreated=7±0.32 μm: treated=2.7±0.08 μm: Mean±SEM: p<0.0001, One-Way ANOVA: FIG. 19).

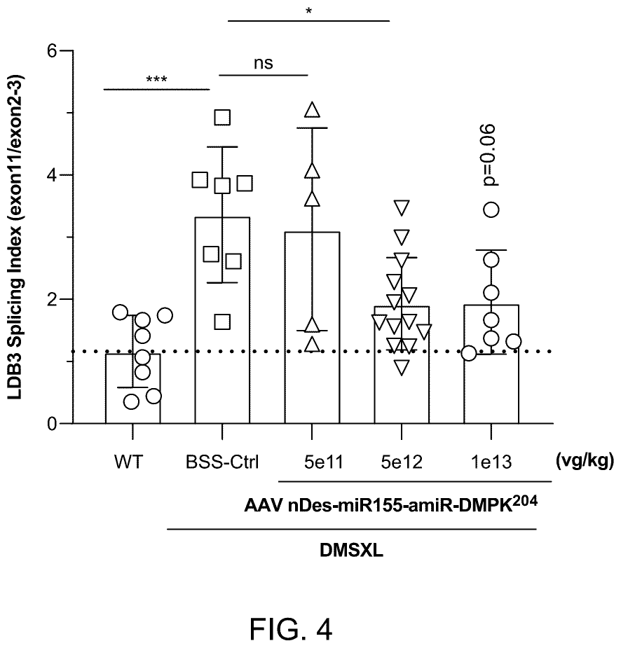

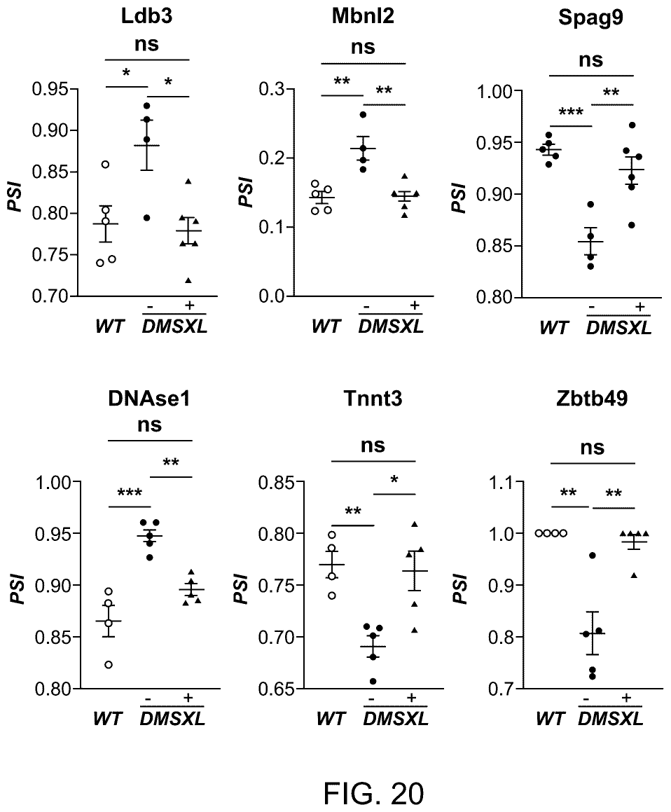

Next, the consequences of DMPK suppression on characteristic DM1 phenotypes, such as splicing abnormalities, was investigated. To determine whether treatment could improve the splicing phenotype in the mice, hearts and tibialis anterior (TA) muscles were collected and RNA-Seq analysis was performed in wild-type, buffer negative control, and treated mic (Shen et al. (2014) Proc Natl Acad Sci 111 (51): E5593-E5601; Park et al. (2013) Methods Mol Biol 1038:171-9; Shen et al. (2012) Nucleic Acids Res 40 (8):e61). Global alterations in alternative splicing events in the heart and TA were found indicating molecular changes in disease and with treatment. Among the genes that were altered in the heart of the DMSXL mice were Ldb3, Mbnl2, and Spag9 (FIG. 20). Aberrant splicing of LIM domain binding 3 (Ldb3), resulting in inclusion of exon 11 in Ldb3 transcripts, has been demonstrated as DM1-specific phenotype resulting from sequestration of RNA splicing machinery by CUG repeat RNA (Yamashita et al. 2014. Neurobiol Dis. 69:200-5). A significant reduction in the Ldb3 transcript with the inclusion of exon 11 in the gastrocnemius muscle was observed in mice that were treated with the medium-dose or the high dose of AAV nDes-miR155-amiR-DMPK²⁰⁴, confirming that splicing defects were efficiently corrected in muscle treated with amiR155-DMPK²⁰⁴ (FIG. 4). It has also been shown previously that the persistence of fetal isoforms of these genes contribute to DM1 pathology. In TA, a different, but overlapping set of alternative splicing events were altered when comparing wild-type and DMSXL mice. Among others, three genes demonstrated significant splicing rescue in the treated group-DNAseI, Tnnt3, and Zbtb49 (FIG. 20). For these transcripts, both in the heart as well as in the TA, there were no significant differences between wild-type and the treated DMSXL mice, suggesting normal splicing restoration following treatment.

In addition to these molecular corrections, the efficacy of AAV nDes-miR155-amiR-DMPK²⁰⁴ was then measured in terms of physiological and functional manifestations of the disease.

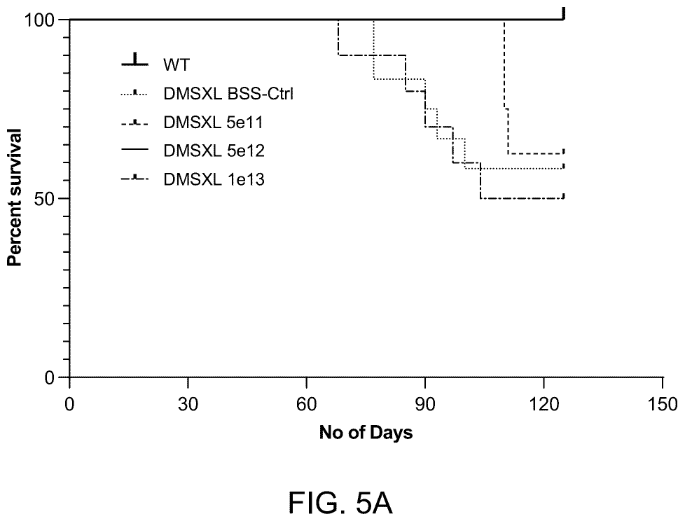

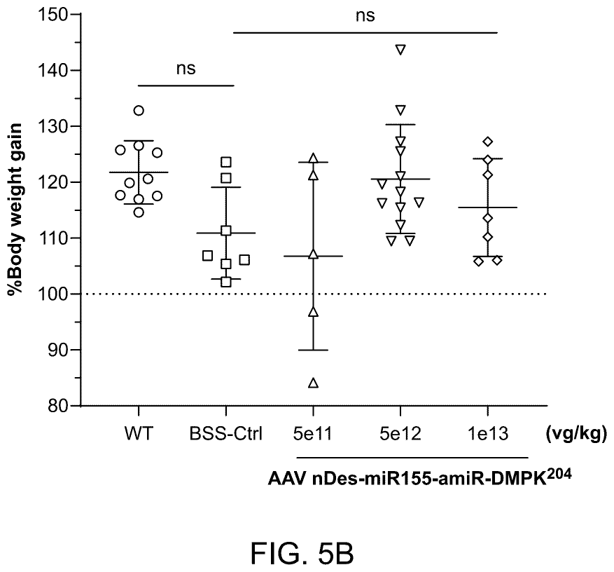

To determine whether the treatment can improve survival and attenuate loss in body weight, DMSXL mice were treated with AAV nDes-miR155-amiR-DMPK²⁰⁴ at three different doses and monitored the survival and body weight. Improved body weight and survival rate were observed after eight weeks of treatment with medium and high doses. On the other hand, no improvement was observed with low dose or Balanced Salt Solution (BSS) control (FIGS. 5A and 5B).

Next, the efficacy of AAV nDes-miR155-amiR-DMPK²⁰⁴ was measured in terms of functional manifestations of disease such as prevention of myotonia and cardiac abnormalities. Electromyography measurements revealed a significant decrease in myotonia in mice treated with AAV nDes-miR155-amiR-DMPK²⁰⁴ (Table 2). In particular, after treatment, only 7.6% of the animals which were treated with the medium dose had myotonia. In contrast, >50% of mice in control (treated with BSS) or low dose groups had persistent myotonia (scores 1).

TABLE 2
Number of DMSXL DM1 mice with myotonia after
treatment with AAV nDes-miR155- amiR-DMPK²⁰⁴.

| | Number of Animals with | | | |
|---|---|---|---|---|
| Group | Myotonia (gastrocnemius) | | | |
| WT | 0/10 | | | |
| | | {close oversize bracket} | | |
| DMSXL-Ctrl (untreated) | 4/7 | | | |
| DMSXL-5E11 VG/Kg | 3/5 | | | P < 0.05 |
| (low dose) | | | {close oversize bracket} | |
| DMSXL-5E12 VG/Kg | 1/13 | | | |
| (medium dose) | | | | |
| DMSXL-5E13 VG/Kg | 1/7 | | | |
| (high dose) | | | | |

Myotonic discharges were graded on a 4-point scale: 0, no myotonia: 1, occasional myotonic discharge in less than 50% of needle insertions: 2, myotonic discharge in greater than 50% of needle insertions: 3: myotonic discharge with nearly every insertion.

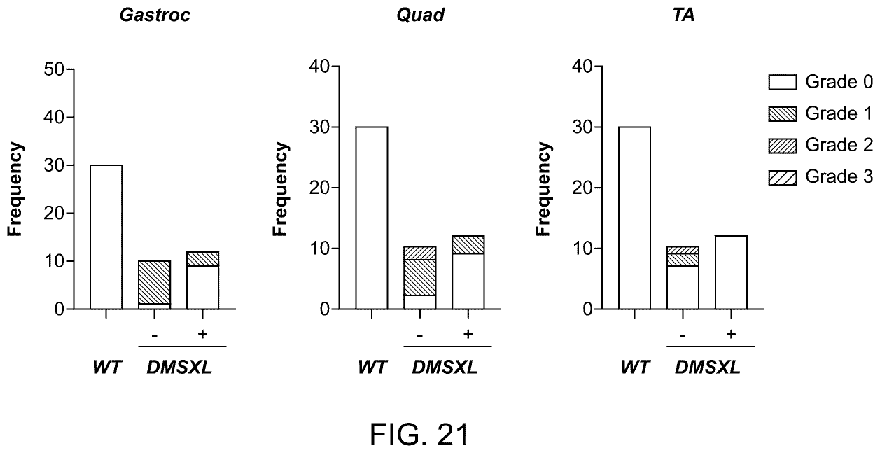

The number of grade 0) events vs grade 1-3 events were compared across treatment groups (wild-type, vehicle control in DMSXL mice, and a single intravenous dose of amiR155-DMPK²⁰⁴ in DMSXL mice) (FIG. 21). As expected, all wild-type mice scored 0 in all three muscles analyzed. In the DMSXL mice treated with vehicle, there was a significant reduction of grade 0 events recorded in all three muscles analyzed (Gastroc, Grade 0): WT=30; DMSXL Vehicle=1: Grade 1-3: WT=0), DMSXL Vehicle=9; Fisher's exact test, Bonferroni-Holm adjusted p-value<0.0001: Quad, Grade 0): WT=30: DMSXL Vehicle=2: Grade 1-3: WT=0), DMSXL Vehicle=8: Fisher's exact test. Bonferroni-Holm adjusted p-value<0.0001: TA, Grade 0 WT=30: DMSXL Vehicle=7: Grade 1-3: WT=0 DMSXL Vehicle=3: Fisher's exact test, Bonferroni-Holm adjusted p-value=0.036). However, treatment with both doses significantly improved the myotonia phenotype in the gastrocnemius and showed a positive tendency of improvement in the other two muscles, compared to the Vehicle group (Gastroc: DMSXL 9 X1013, Grade 0=9); Grade 1−3=3; Fisher's exact test, Bonferroni-Holm adjusted p-value=0.0041: DMSXL 1.8 X1014, Grade 0=15: Grade 1−3=6; Fisher's exact test, Bonferroni-Holm adjusted p-value=0.0041: Quad: DMSXL 9 X1013, Grade 0=9; Grade 1−3=3: Fisher's exact test, Bonferroni-Holm adjusted p-value=0.059; DMSXL 1.8 X1014, Grade 0=13: Grade 1−3=8; Fisher's exact test, Bonferroni-Holm adjusted p-value=0.059; TA: DMSXL 9 X1013 Grade 0=12: Grade 1-3=0 Fisher's exact test, Bonferroni-Holm adjusted p-value=0.155: DMSXL 1.8 X1014, Grade 0=19; Grade 1−3=2: Fisher's exact test, Bonferroni-Holm adjusted p-value=0.295). To determine if treatment with the AAV capsid containing the miR155-DMPK²⁰⁴ construct could correct this defect, the grade of myotonic events in the gastrocnemius, quadriceps, and TA, via EMG was measured. While wild-type mice exhibited no myotonic events in any of the muscles analyzed, the majority of the DMSXL untreated mice showed events with grades between 1 and 2, and most of the treated mice showed myotonia grades between 0) and 1 (FIG. 21)

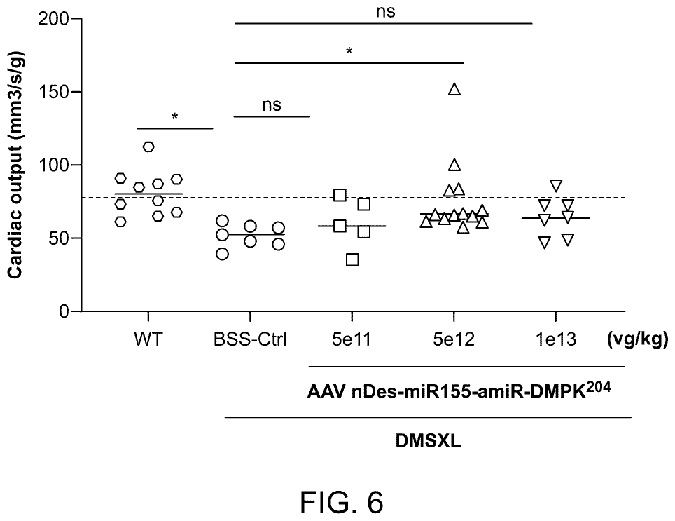

Another clinical hallmark of the disease is defects in cardiac function. Conduction defects are found in up to 75% of DM1 adult patients and cardiac arrhythmias are a leading cause of death. Cardiac function of the DMSXL mice was also monitored using surface echocardiogram 8 weeks post-treatment along with the skeletal muscle function. AAV nDes-miR155-amiR-DMPK²⁰⁴ improved cardiac output as compared to BSS treated controls. Significant improvement in cardiac output was noted in medium dose group (5e12 vg/kg) after 8 weeks of treatment (FIG. 6).

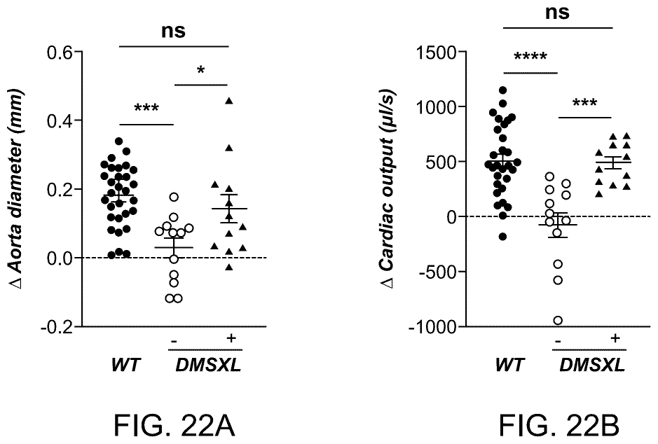

Echocardiographic analysis of the DSMXL hearts showed that the mice exhibited a narrowed aorta compared to their wild-type littermates (FIG. 22, p<0.001, One-way ANOVA). Treatment with 9×10¹³ vg/kg amiR-DMPK²⁰⁴, however, significantly increased aortic diameter (p<0.05). Treated DMSXL mice were not significantly different from wild type (FIG. 22A). Moreover, it was found that DMSXL mice had decreased ascending aortic blood velocity (cardiac output deficiency: FIG. 22B), compared to wild-type (p<0.0001), and this defect was also corrected with treatment (p<001).

### Example 5: The AAVrh74N502I Capsid has Improved Muscle Transduction and Reduced Liver Transduction

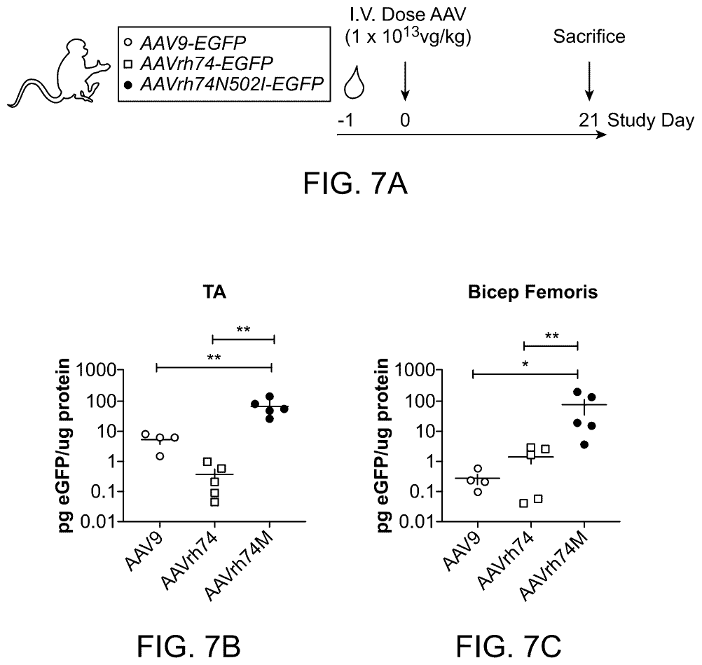

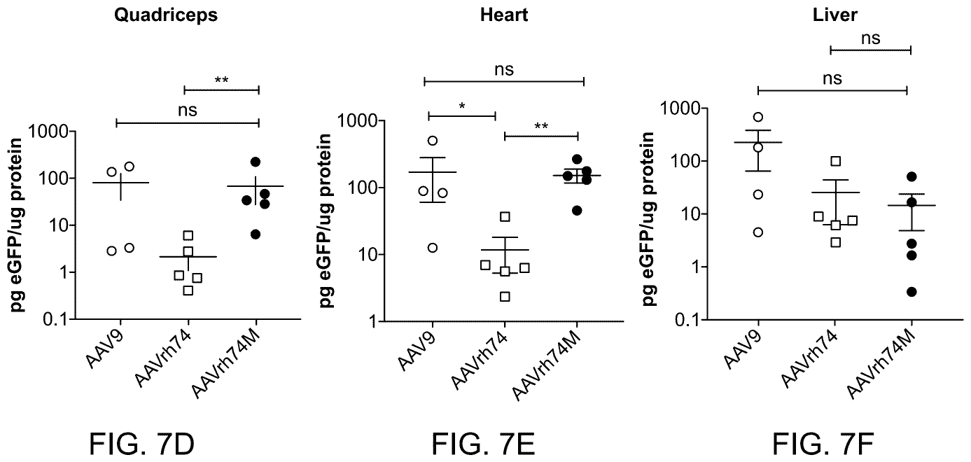

An experiment was performed to test the transduction efficiency of AAV capsids containing the AAVrh74N502I VP1 capsid protein (SEQ ID NO: 50) in various tissues in non-human primates. An outline of the experiment is shown in FIG. 7A. Non-human primates were treated intravenously with 1×10¹³ vg/kg of either AAV9, AAVrh74, or AAVrh74N502I capsids, each containing an eGFP expression cassette. Twenty-one days after treatment, the animals were sacrificed, and the levels of eGFP expression in the tibialis anterior (TA), bicep femoris, quadriceps, heart, and liver were measured.

The capsids containing the AAVrh74N502I capsid protein had improved muscle transduction (FIGS. 7B-7E) and reduced liver transduction (FIG. 7F) in the non-human primate compared to comparator capsids. (Table 3).

TABLE 3
Increase in eGFP levels in tissues of non-human primates
treated with AAVrh74N502I capsids relative to those
treated with AAV9 and AAVrh74 capsids.

| Tissue | AAV9 | AAVrh74 |
|---|---|---|
| Tibialis Anterior | +13x | +178x |
| Bicep Femoris | +303x | +56x |
| Quadriceps | NS* | +32x |
| Heart | NS | +13x |
| Liver | −15x | −2x |

*NS = not significant

### Example 6: Evaluation of AA Vrh74N502I nDes-miR155-amiR-DMPK²⁰⁴ Target Engagement in DMSXL Mice Model

To determine the most efficacious dose, the delivery of two separate doses of AAVrh74N502I capsid were investigated. AAV encoding the expression cassette for amiR-DMPK²⁰⁴ with the miR155 scaffold (miR 155-amiR-DMPK²⁰⁴) was evaluated in a dose-escalation study. Eight-week-old DMSXL mice were injected intravenously with 9×10¹³ vector genomes (vg)/kg, and 1.8×10¹⁴ vg/kg, corresponding to low, and high doses. Mice were euthanized 8 weeks post gene transfer, and DMPK suppression and amiR-DMPK²⁰⁴ expression levels were measured by small RNA TaqMan, and mRNA input levels was normalized to u6 small nuclear RNA.

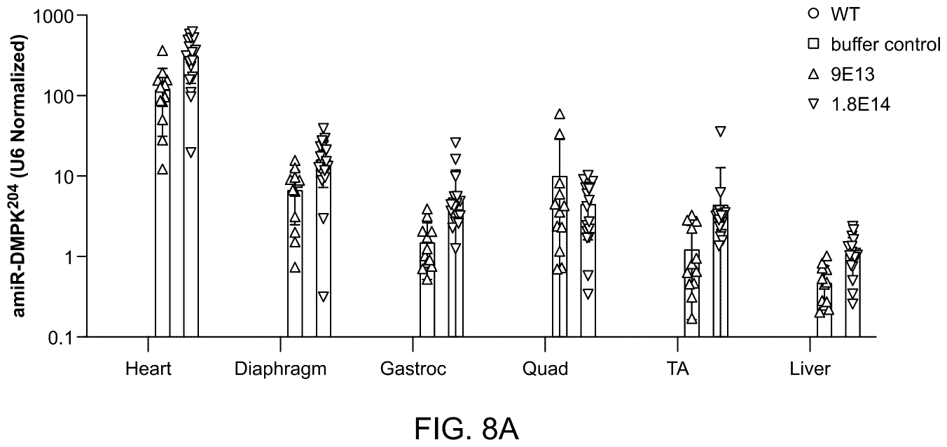

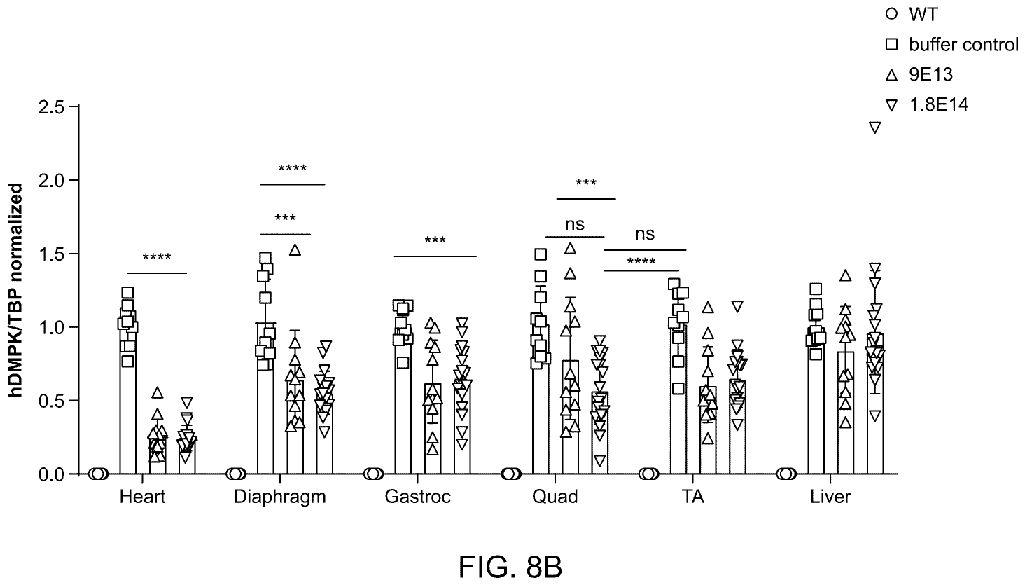

The expression of amiR-DMPK²⁰⁴ was observed in a dose-dependent manner (FIG. 8A) and resulted in a dose-dependent reduction of total DMPK expression (FIG. 8B) in multiple tissues. Overall, it was observed that ˜10 amiR-DMPK²⁰⁴ copies/U6 was sufficient to reduce DMPK by ≥50% in the heart and diaphragm, an amount which may be sufficient to treat DM1 patients.

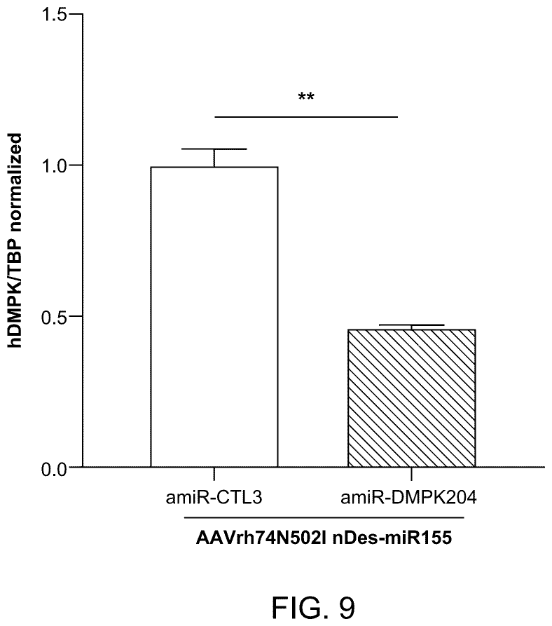

Finally, the lead vector, nDes-miR155-amiR-DMPK²⁰⁴, in the context of a myotropic capsid, AAVrh74N502I (SEQ ID NO: 50), was shown to have potent in vitro activity in the cardiomyocytes derived DM1 iPSCs (FIG. 9).

### Example 7: Evaluation of AA Vrh74N502I nDes-miR155-amiR-DMPK²⁰⁴ on Transcriptome

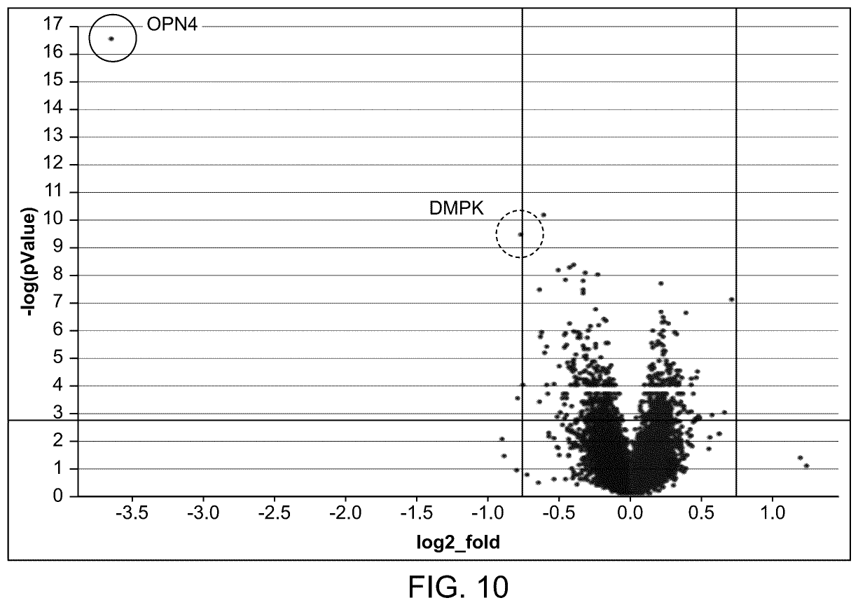

To determine whether amiR-DMPK²⁰⁴ treatment had any major effect on the transcriptome, genome-wide RNA sequencing (RNA-seq) was performed, comparing amiR-DMPK²⁰⁴-treated to CTL3 (scramble miRNA) by transfecting CBA miR155-amiR-DMPK²⁰⁴ plasmid into HEK293 cell lines. To evaluate whether the observed non-DMPK gene expression changes were due to the off-target effects of the amiR-DMPK²⁰⁴, enrichment for seed complementarity in significantly downregulated targets was evaluated. There were four differentially expressed genes OPN4 (12.5 fold), DMPK (1.7 fold), KRTAP21-2 (1.7 fold), C8ORF44-SGK3 (1.7 fold) whose 3′ UTRs contain the TTCGAC seed complement, using a 5% false discovery rate (FDR) significance threshold (Table 4). With a 1% FDR, only OPN4 (12.5 fold), DMPK (1.7 fold), showing differential expression (FIG. 10). As expected. DMPK was one of the most significantly impacted mRNA levels (44% silencing). Overall, minimal off target effects were observed following over expression of amiR-DMPK²⁰⁴ in HEK293 cells.

TABLE 4

| | log2 fold | Fold | | | |
|---|---|---|---|---|---|
| | 204 vs | 204 vs | | FDR_BH | FDR_BH |
| Gene Name | CTL3 | CTL3 | P value | <0.05 | <0.01 |
| OPN4 | −3.64 | 12.5 | 2.7746E−17 | 5E−13 | yes |
| DMPK | −0.77 | 1.7 | 3.4308E−10 | 2E−06 | yes |
| KRTAP21-2 | −0.76 | 1.7 | 0.0001 | 0.0103 | |
| C8orf44- | −0.79 | 1.7 | 0.0003 | 0.0191 | |
| SGK3 | | | | | |

### Example 8: Dose Range Finding Study to Explore Biodistribution and Activity of AAVrh74N502I nDes-miR155-amiR-DMPK²⁰⁴ in Non-Human Primates

To determine the biodistribution and activity of AAVrh74MN502I nDes-miR155-amiR-DMPK²⁰⁴, a single intravenous infusion (IV) dose was administered to cynomolgus monkeys. The study duration was for 12 weeks after the single injection.

Sixteen total cynomolgous monkeys (8 male, 8 female; 24 to 48 months old) were dosed via IV in the saphenous vein once on day 1 of the study. Doses are indicated in Table 5 below. The animals were grouped (2 males and 2 females per group) into four different categories based on the dose level (vg/kg): formulation buffer (Group 1); 5×10¹³ vg/kg (Group 2); 1×10¹⁴ vg/kg (Group 3); and 2×10¹⁴ vg/kg (Group 4). After 12 weeks, the animals were sacrificed and tissue was harvested for analysis.

TABLE 5

| | | Dose | Dose | | |
|---|---|---|---|---|---|
| | Test Article or | Level | Concentration | Number of Animals | |
| Group | Control Article | (vg/kg) | (vg/mL) | Males | Females |
| 1 | Formulation Buffer | 0 | 0 | 2 | 2 |
| 2 | AAVrh74N502I nDes- | 5 × 10¹³ | 5 × 10¹² | 2 | 2 |
| | miR155- | | vg/ml | | |
| | amiR-DMPK²⁰⁴ | | | | |
| 3 | AAVrh74N502I nDes- | 1 × 10¹⁴ | 1 × 10¹³ | 2 | 2 |
| | miR155- | | vg/ml | | |
| | amiR-DMPK²⁰⁴ | | | | |
| 4 | AAVrh74N502I nDes- | 2 × 10¹⁴ | 2 × 10¹³ | 2 | 2 |
| | miR155- | | vg/ml | | |
| | amiR-DMPK²⁰⁴ | | | | |

Harvested tissue was mechanically homogenized for RNA and DNA extraction, and samples were analyzed with digital PCR (dPCR).

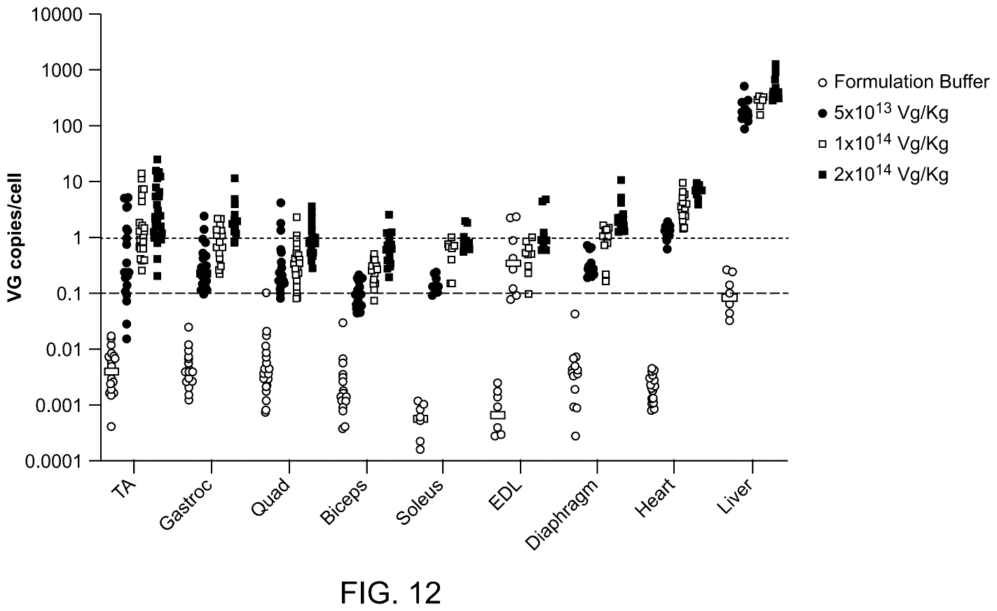

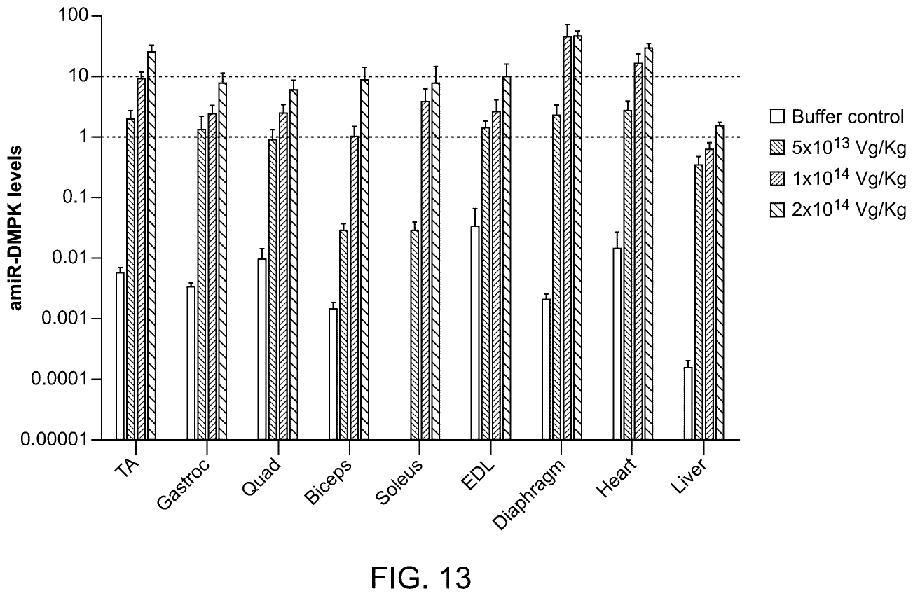

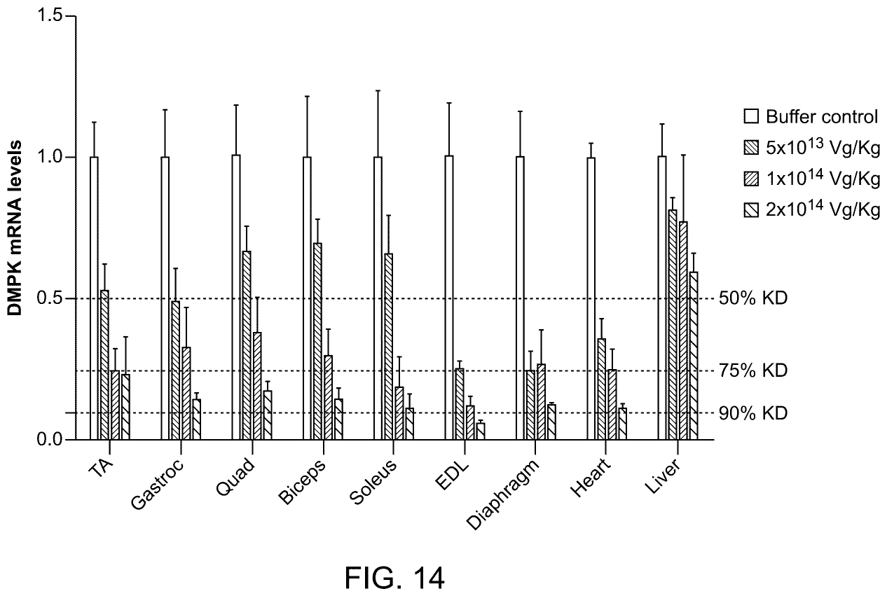

Dose-dependent biodistribution and activity of AAVrh74N502I nDes-miR155-amiR-DMPK²⁰⁴ was demonstrated in several muscle and non-muscle tissues. Several skeletal muscles were analyzed (tibialis anterior muscle (TA); gastrocnemius; quadricep; biceps; soleus; extensor digitorum longus (EDL); diaphragm) as well as heart muscle and liver tissue. Viral genome copies were found in all tissues tested and the number of copies/cell in each tissue were in a dose-dependent manner (FIG. 12). The expression of amiR-DMPK expression (FIG. 13) and the downregulation of DMPK (FIG. 14) was also in dose-dependent manner in various muscle tissues, heart tissue, and liver tissue. The dose-dependent reduction of DMPK expression was found to be up to 90% reduced as compared to a control group. All doses tested in the animals were found to be safe and well-tolerated by the animals.

### Example 9: Rescue of Splicing Defects Following Treatment with amiR-DMPK²⁰⁴b

DMPK-mediated splicing defects are a molecular hallmark in DM1 tissue. To determine whether amiR-DMPK²⁰⁴ treatment had an effect on these splicing defects, a targeted splicing analysis was performed using both RNA sequencing (RNA-seq) and the Nanostring platform, focusing on 36 genes (Tanner et al. 2021. Nucleic Acids Res. 49 (4): 2240-2254. doi: 10.1093/nar/gkab022).

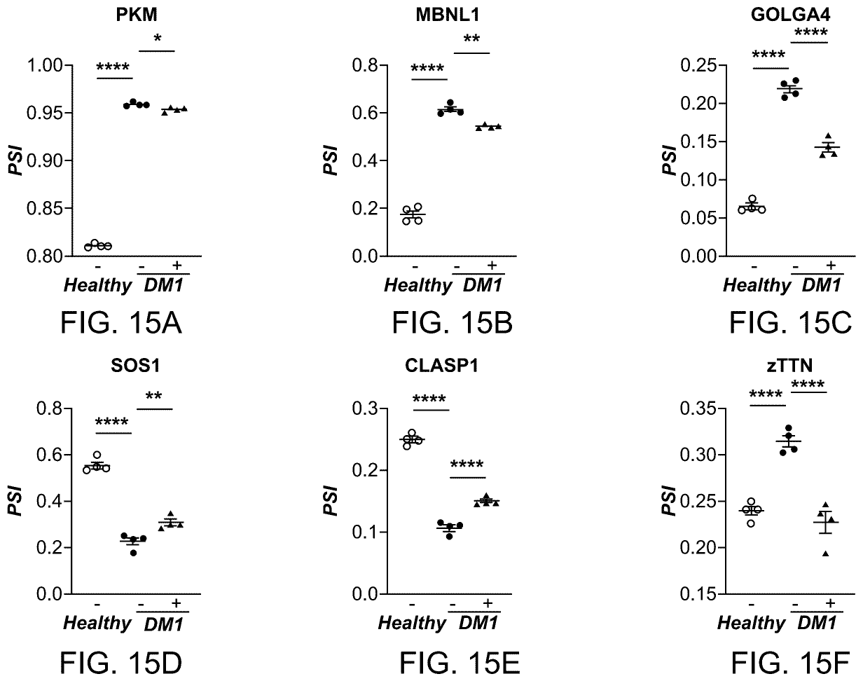

Data between the two platforms were comparable. Immortalized human DM1 myotubes were transduced with AAV2-nDesmin-nDES-miR155-DMPK²⁰⁴ and were compared to both untreated DM1 myotubes and healthy control myotubes. 25 out of the 36 genes showed a statistically significant modification of their splicing prior to treatment. Six of the 25 genes (MBNL1, SOS1, PKM, zTTN, GOLGA4, and CLASP1) demonstrated a significant recovery in the treated cells, compared to untreated (FIG. 15). Overall, the data demonstrates that treatment of human myotubes with amiR-DMPK²⁰⁴ was able to at least partially rescue the splicing defects caused by DMPK mutations.

### Example 10: AAVrh74N502I nDes-miR155-amiR-DMPK²⁰⁴ Up to 2×10¹⁴ vg/kg is Well Tolerated in Non-Human Primates

Tissue sections from animals treated with AAVrh74N502I nDes-miR155-amiR-DMPK²⁰⁴ were evaluated for tolerability to the AAV construct. All animals in the study were monitored during the in-life phase including detailed cage side observations, body weight, food consumption, and vital signs measurements.

No test article-related macroscopic observations were noted. Test article-related minimal individual hepatocyte necrosis was noted in the liver of one male administered 1×10¹⁴ vg/kg and one female administered 2×10¹⁴ vg/kg (Table 6). Minor test article-related clinical chemistry effects were identified in animals administered a single dose ≥1×10¹⁴, characterized by minimally to mildly increased aspartate aminotransferase (AST), alanine aminotransferase (ALT), and glutamate dehydrogenase activities (GDH) (Table 7). Enzyme increases were observed only at early timepoints, fully reversed to baseline values by Day 43, and lacked correlative microscopic liver findings in affected animals except for the finding of minimally individual hepatocyte necrosis in one female administered 2×10¹⁴ vg/kg. No evidence of a liver effect was identified in animals administered 5×10¹³ vg/kg. No test article-related effects were identified on hematology or coagulation parameters at any dose level. These changes were considered not adverse based on a general small magnitude of change, minimal severity and incidence, and/or lack of dose-relatedness.

TABLE 6
Incidence and severity of test article-related microscopic
findings in animals administered amiR-DMPK²⁰⁴.

| | rAAVmiR | | | | | | | |
|---|---|---|---|---|---|---|---|---|
| | Sex | | | | | | | |
| | Males | | | | Females | | | |
| Dose level | | 5 × | 1 × | 2 × | | 5 × | 1 × | 2 × |
| (vg/kg) | 0 | 10¹³ | 10¹⁴ | 10¹⁴ | 0 | 10¹³ | 10¹⁴ | 10¹⁴ |
| Liver | 2 | 2 | 2 | 2 | 2 | 2 | 2 | 2 |
| Number | | | | | | | | |
| examined | | | | | | | | |
| Necrosis, | 0 | 0 | 1 | 0 | 0 | 0 | 0 | 1 |
| individual | | | | | | | | |
| hepatocyte | | | | | | | | |
| Minimal | | | | | | | | |

TABLE 7
Test article-related liver enzyme changes in
selected animals administered amiR-DMPK²⁰⁴.

| | Enzyme Absolute Activity, U/L (Fold change compared with predose)^{a} | | | | | | | | |
|---|---|---|---|---|---|---|---|---|---|
| | | Dosing phase | | | | | | | |
| Day | Predose −41 | 4 | 8 | 15 | 29 | 30 | 31 | 43 | 85 |

Animal P0202, Male administered 1 × 10¹⁴ vg/kg

| | | | | | | | | | |
|---|---|---|---|---|---|---|---|---|---|
| AST | 33 | 133 | 38 | 35 | — | 35 | — | 37 | 40 |
| | | (4.0X) | | | | | | | |
| ALT | 32 | 74 | 126 | 52 | — | 34 | — | 31 | 35 |
| | | (2.3X) | (3.9X) | | | | | | |
| GDH | 22 | 89 | 92 | 35 | ? | 28 | — | 26 | 28 |
| | | (4.0X) | (4.2X) | | | | | | |

Animal P0601, Female administered 1 × 10¹⁴ vg/kg

| | | | | | | | | | |
|---|---|---|---|---|---|---|---|---|---|
| AST | 33 | 102 | 52 | 40 | — | 32 | — | 29 | 32 |
| | | (3.1X) | | | | | | | |
| ALT | 84 | 180 | 171 | 113 | — | 59 | — | 60 | 59 |
| | | (2.1X) | (2.0X) | | | | | | |
| GDH | 24 | 59 | 68 | 36 | — | 16 | — | 18 | 19 |
| | | (2.5X) | (2.8X) | | | | | | |

Animal P0401, Male administered 2 × 10¹⁴ vg/kg

| | | | | | | | | | |
|---|---|---|---|---|---|---|---|---|---|
| AST | 48 | 91 | 198 | 96 | — | — | 60 | 52 | 40 |
| | | | (4.1X) | (2.0X) | | | | | |
| ALT | 34 | 63 | 197 | 80 | — | — | 50 | 35 | 26 |
| | | | (5.8X) | (2.4X) | | | | | |
| GDH | 35 | 45 | 87 | 45 | ? | ? | 40 | 34 | 32 |
| | | | (2.5X) | | | | | | |

Animal P0302, Male administered 2 × 10¹⁴ vg/kg

| | | | | | | | | | |
|---|---|---|---|---|---|---|---|---|---|
| AST | 48 | 688 | 58 | 80 | — | — | 57 | 52 | 50 |
| | | (14.3X) | | | | | | | |
| ALT | 83 | 686 | 306 | 99 | — | — | 66 | 64 | 61 |
| | | (8.3X) | (3.7X) | | | | | | |
| GDH | 45 | 92 | 75 | 37 | | | 32 | 31 | 30 |
| | | (>2.0X) | | | | | | | |

Animal P0701, Female administered 2 × 10¹⁴ vg/kg

| | | | | | | | | | |
|---|---|---|---|---|---|---|---|---|---|
| AST | 43 | 136 | 82 | 110 | — | — | 51 | 54 | 61 |
| | | (3.2X) | | (2.6X) | | | | | |
| ALT | 40 | 119 | 109 | 109 | — | — | 41 | 59 | 62 |
| | | (3.0X) | (2.7X) | (2.7X) | | | | | |
| GDH | 27 | 44 | 54 | 61 | — | — | 23 | 36 | 46 |
| | | | (2.0X) | (2.3X) | | | | | |

Animal P0702, Female administered 2 × 10¹⁴ vg/kg

| | | | | | | | | | |
|---|---|---|---|---|---|---|---|---|---|
| AST | 35 | 58 | 65 | 51 | — | — | 34 | 43 | 35 |
| ALT | 45 | 69 | 99 | 60 | — | — | 34 | 61 | 42 |
| | | | (2.2X) | | | | | | |
| GDH | 23 | 32 | 53 | 26 | — | — | 16 | 28 | 22 |
| | | | (2.3X) | | | | | | |

### Sequences

All polypeptide sequences are presented as N-terminal to C-terminal unless indicated otherwise. All nucleic acid sequences are presented as 5′ to 3′ unless indicated otherwise.

### Byrne Desmin Enhancer Sequence

(SEQ ID NO: 21)
CACCCATGCCTCCTCAGGTACCCCCTGCCCCCCACAGCTCCTCTCCTGTG
CCTTGTTTCCCAGCCATGCGTTCTCCTCTATAAATACCCGCTCTGGTATT
TGGGGTTGGCAGCTGTTGCTGCCAGGGAGATGGTTGGGTTGACATGCGGC
TCCTGACAAAACACAAACCCCTGGTGTGTGTGGGCGTGGGTGGTGTGAGT
AGGGGGATGAATCAGGGAGGGGGCGGGGGACCCAGGGGGCAGGAGCCACA
CAAAGTCTGTGCGGGGGGGGAGCGCACATAGCAATTGGAAACTGAAAGCT
TATCAGACCCTTTCTGGAAATCAGCCCACTGTTTATAAACTTGAGGCCCC
ACCCTCGA

Source: Li and Paulin, et. al. 1991. “High level desmin expression depends on a muscle-specific enhancer.” Journal of Biol Chem. 266.10:6562-6570.

Homo sapiens desmin locus control region (DES-LCR) on chromosome 2 (NCBI Reference Sequence: NG_046330.1)

Byrne enhancer sequence corresponds to 17767-18125 in Ref Seq.

### Paulin Desmin Enhancer Sequence

(SEQ ID NO: 22)
CCCCCTGCCCCCCACAGCTCCTCTCCTGTGCCTTGTTTCCCAGCCATGCG
TTCTCCTCTATAAATACCCGCTCTGGTATTTGGGGTTGGCAGCTGTTGCT
GCCAGGGAGATGGTTGGGTTGACATGCGGCTCCTGACAAAACACAAACCC
CTGGTGTGTGTGGGCGTGGGTGGTGTGAGTAGGGGGATGAATCAGGGAGG
GGGCGGGGGACCCAGGGGGCAGGAGCCACACAAAGTCTGTGCGGGGGTGG
GAGCGCACATAGCAATTGGAAACTGAA

Source: Li and Paulin, et. al. 1991. “High level desmin expression depends on a muscle-specific enhancer.” Journal of Biol Chem. 266.10:6562-6570.

Homo sapiens desmin locus control region (DES-LCR) on chromosome 2 (NCBI Reference Sequence: NG_046330.1)

Paulin enhancer sequence corresponds to 17787-18063 in Ref Seq

### Paulin Desmin Promoter Sequence (−228 to +75)

(SEQ ID NO: 23)
CTGCAGACCTGCTTGCTGCCTGCCCTGGCGAAGGATTGGCAGGCTTGCCC
GTCACAGGACCCCCGCTGGCTGACTCAGGGGCGCAGGCCTCTTGCGGGGG
AGCTGGCCTCCCCGCCCCCACGGCCACGGGCCGCCCTTTCCTGGCAGGAC
AGCGGGATCTTGCAGCTGTCAGGGGAGGGGAGGCGGGGGCTGATGTCAGG
AGGGATACAAATAGTGCCGACGGCTGGGGGCCCTGTCTCCCCTCGCCGCA
TCCACTCTCCGGCCGGCCGCCTGCCCGCCGCCTCCTCCGTGCGCCCGCCA
GCCTCGCCCG

There is a one basepair difference from published sequence (C instead of an A, shown in bold and underlined).

Source: Li and Paulin, et. al. 1991. “High level desmin expression depends on a muscle-specific enhancer.” Journal of Biol Chem. 266.10:6562-6570.

Homo sapiens desmin locus control region (DES-LCR) on chromosome 2 (NCBI Reference Sequence: NG_046330.1)

Paulin promoter sequence corresponds to 18535-18844 in Ref Seq

Complete nDes Promoter Sequence

(SEQ ID NO: 12)
CACCCATGCCTCCTCAGGTACCCCCTGCCCCCCACAGCTCCTCTCCTGTG
CCTTGTTTCCCAGCCATGCGTTCTCCTCTATAAATACCCGCTCTGGTATT
TGGGGTTGGCAGCTGTTGCTGCCAGGGAGATGGTTGGGTTGACATGCGGC
TCCTGACAAAACACAAACCCCTGGTGTGTGTGGGCGTGGGTGGTGTGAGT
AGGGGGATGAATCAGGGAGGGGGCGGGGGACCCAGGGGGCAGGAGCCACA
CAAAGTCTGTGCGGGGGTGGGAGCGCACATAGCAATTGGAAACTGAAAGC
TTATCAGACCCTTTCTGGAAATCAGCCCACTGTTTATAAACTTGAGGCCC
CACCCTCGAGGTACCCCCTGCCCCCCACAGCTCCTCTCCTGTGCCTTGTT
TCCCAGCCATGCGTTCTCCTCTATAAATACCCGCTCTGGTATTTGGGGTT
GGCAGCTGTTGCTGCCAGGGAGATGGTTGGGTTGACATGCGGCTCCTGAC
AAAACACAAACCCCTGGTGTGTGTGGGCGTGGGTGGTGTGAGTAGGGGGA
TGAATCAGGGAGGGGGCGGGGGACCCAGGGGGCAGGAGCCACACAAAGTC
TGTGCGGGGGTGGGAGCGCACATAGCAATTGGAAACTGAAAGCTTCTGCA
GACCTGCTTGCTGCCTGCCCTGGCGAAGGATTGGCAGGCTTGCCCGTCAC
AGGACCCCCGCTGGCTGACTCAGGGGCGCAGGCCTCTTGCGGGGGAGCTG
GCCTCCCCGCCCCCACGGCCACGGGCCGCCCTTTCCTGGCAGGACAGCGG
GATCTTGCAGCTGTCAGGGGAGGGGAGGCGGGGGCTGATGTCAGGAGGGA
TACAAATAGTGCCGACGGCTGGGGGCCCTGTCTCCCCTCGCCGCATCCAC
TCTCCGGCCGGCCGCCTGCCCGCCGCCTCCTCCGTGCGCCCGCCAGCCTC
GCCCG

Source: Li and Paulin, et. al. 1991. “High level desmin expression depends on a muscle-specific enhancer.” Journal of Biol Chem. 266.10:6562-6570.

### Rabbit β-Globin Intron

(SEQ ID NO: 13)
GTGAGTTTGGGGACCCTTGATTGTTCTTTCTTTTTCGCTATTGTAAAATT
CATGTTATATGGAGGGGGCAAAGTTTTCAGGGTGTTGTTTAGAATGGGAA
GATGTCCCTTGTATCACCATGCATGGACCCTCATGATAATTTTGTTTCTT
TCACTTTCTACTCTGTTGACAACCATTGTCTCCTCTTATTTTCTTTTCAT
TTTCTGTAACTTTTTCGTTAAACTTTAGCTTGCATTTGTAACGAATTTTT
AAATTCACTTTTGTTTATTTGTCAGATTGTAAGATCCCATCGATTCCAAT
CAGGGTATATTATATTGTACTTCAGCACAGTTTTAGAGAACAATTGTTAT
AATTAAATGATAAGGTAGAATATTTCTGCATATAAATTCTGGCTGGCGTG
GAAATATTCTTATTGGTAGAAACAACTACATCCTGGTCATCATCCTGCCT
TTCTCTTTATGGTTACAATGATATACACTGTTTGAGATGAGGATAAAATA
CTCTGAGTCCAAACCGGGCCCCTCTGCTAACCATGTTCATGCCTTCTTCT
TTTTCCTACAG

5′ arm of rabbit β-globin intron

(SEQ ID NO: 14)
GTGAGTTTGGGGACCCTTGATTGTTCTTTCTTTTTCGCTATTGTAAAATT
CATGTTATATGGAGGGGGCAAAGTTTTCAGGGTGTTGTTTAGAATGGGAA
GATGTCCCTTGTATCACCATGCATGGACCCTCATGATAATTTTGTTTCTT
TCACTTTCTACTCTGTTGACAACCATTGTCTCCTCTTATTTTCTTTTCAT
TTTCTGTAACTTTTTCGTTAAACTTTAGCTTGCATTTGTAACGAATTTTT
AAATTCACTTTTGTTTATTTGTCAGATTGTAAGATCCCATCGATTC

Source: Oryctolagus cuninculus hemoglobin, beta (HBB2) Gene ID 100009084

The sequence of the present invention has an additional CATG (shown in bold and underlined) that is not present in Gene ID 100009084.

### 3′ Arm of Rabbit β-Elobin Intron

(SEQ ID NO: 15)
CAATCAGGGTATATTATATTGTACTTCAGCACAGTTTTAGAGAACAATT
GTTATAATTA
AATGATAAGGTAGAATATTTCTGCATATAAATTCTGGCTGGCGTGGAAA
TATTCTTATT
GGTAGAAACAACTACATCCTGGTCATCATCCTGCCTTTCTCTTTATGGT
TACAATGATA
TACACTGTTTGAGATGAGGATAAAATACTCTGAGTCCAAACCGGGCCCC
TCTGCTAACCATGTTCATGCCTTCTTCTTTTTCCTACAG

Source: Oryctolagus cuninculus hemoglobin, beta (HBB2) Gene ID 100009084

The sequence of the present invention has two T residues (shown in bold and underlined above) instead of two C residues in the Gene ID 100009084.

miR155-DMPK²⁰⁴ Sequences:

5′ miR155 flanking sequence
RNA sequence
(SEQ ID NO: 9)
CUGGAGGCUUGCUGAAGGCUGUAUGCU
DNA sequence
(SEQ ID NO: 40)
CTGGAGGCTTGCTGAAGGCTGTATGCT
Source: BLOCK-iT ™ Pol II miR RNAi Expression
Vector Kits catalog #K493500

The engineered pre-miRNA sequence structure is based on the murine miR-155 sequence (Lagos-Quintana et al., 2002, Current Biology, 12:9, 735-739).

amiR-DMPK²⁰⁴ Guide-DNA
(SEQ ID NO: 4)
AGTCGAAGACAGTTCTAGGGT
miR155 terminal loop-DNA
(SEQ ID NO: 6)
GTTTTGGCCACTGACTGAC

Source: BLOCK-iT™ Pol II miR RNAi Expression Vector Kits catalog #K493500

The engineered pre-miRNA sequence structure is based on the murine miR-155 sequence (Lagos-Quintana et al., 2002, Current Biology, 12:9, 735-739).

amiR-DMPK²⁰⁴ Passenger-DNA
(SEQ ID NO: 5)
ACCCTAGATGTCTTCGATT
amiR-DMPK²⁰⁴ Guide-RNA- miR155 terminal loop-
amiR-DMPK²⁰⁴ Passenger-RNA
(SEQ ID NO: 7)
AGUCGAAGACAGUUCUAGGGUUGUUUUGGCCACUGACUGACACCCUAGA
UGUCUUCGAUU
amiR-DMPK²⁰⁴ Guide-DNA-miR155 terminal loop-
amiR-DMPK²⁰⁴ Passenger-DNA
(SEQ ID NO: 8)
AGTCGAAGACAGTTCTAGGGTTGTTTTGGCCACTGACTGACACCCTAGA
TGTCTTCGATT
3′ miR155 flanking sequence
RNA sequence
(SEQ ID NO: 10)
GACACAAGGCCUGUUACUAGCACUCACAUGGAACAAAUGGCC
DNA sequence
(SEQ ID NO: 41)
GACACAAGGCCTGTTACTAGCACTCACATGGAACAAATGGCC

Source: BLOCK-iT™ Pol II miR RNAi Expression Vector Kits catalog #K493500

The engineered pre-miRNA sequence structure is based on the murine miR-155 sequence (Lagos-Quintana et al., 2002, Current Biology, 12:9, 735-739).

miR155
RNA
(SEQ ID NO: 11)
CUGGAGGCUUGCUGAAGGCUGUAUGCUGACACAAGGCCUGUUACUAGCA
CUCACAUGGAACAAAUGGCC
DNA
(SEQ ID NO: 42)
CTGGAGGCTTGCTGAAGGCTGTATGCTGACACAAGGCCTGTTACTAGCA
CTCACATGGAACAAATGGCC
Full miR155-DMPK²⁰⁴ duplex sequence-RNA
(SEQ ID NO: 24)
CUGGAGGCUUGCUGAAGGCUGUAUGCUGAGUCGAAGACAGUUCUAGGGU
GUUUUGGCCACUGACUGACACCCUAGAUGUCUUCGAUUCAGGACACAAG
GCCUGUUACUAGCACUCACAUGGAACAAAUGGCC
Full miR155-DMPK²⁰⁴ duplex sequence-DNA
(SEQ ID NO: 25)
CTGGAGGCTTGCTGAAGGCTGTATGCTGAGTCGAAGACAGTTCTAGGGT
GTTTTGGCCACTGACTGACACCCTAGATGTCTTCGATTCAGGACACAAG
GCCTGTTACTAGCACTCACATGGAACAAATGGCC

### Minimal BGHpA Sequence

(SEQ ID NO: 16)
TCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAAT
TGCATCGCATTGTCTGAGTAGGTGTCATTCTATTCTGGGGGGTGGGGTG
GGGCAGGACAGCAAGGGGGAGGATTGGGAAGACAATAGC

Source: Bos taurus growth hormone 1 (GH1) mRNA NCBI Ref Seq NM_180996.1

### 1138 bp A1AT Intron Stuffer Sequence (Upstream of Expression Cassette)

(SEQ ID NO: 18)
TACGTACAATTGGGATCCTTCGAACTTGAGAGAAAACATCCCAGGGATT
TACAGATCAC
ATGCAGGCAGGGACCAGCTCAACCCTTCTTTAATGTCATCCAGGGAGGG
GGCCAGGGAT
GGAGGGGAGGGGTTGAGGAGCGAGAGGCAGTTATTTTTGGGTGGGATTC
ACCACTTTTC
CCATGAAGAGGGGAGACTTGGTATTTTGTTCAATCATTAAGAAGACAAA
GGGTTTGTTG
AACTTGACCTCGGGGGGGATAGACATGGGTATGGCCTCTAAAAACATGG
CCCCAGCAGC
TTCAGTCCCTTTCTCGTCGATGGTCAGCACAGCCTTATGCACGGCCTGG
AGGGGAGAGA
AGCAGAGACACGTTGTAAGGCTGATCCCAGGCCTCGAGCAAGGCTCACG
TGGACACCTC
CCAGGAAGCGCTCACTCCCCCTGGACGGCCCTGGCCCTGCACATCCTCT
CCCTCCCTGT
CACATAGGCCTTGCTCCTCCTCAAGGCTTTGGCTGATGGGGCTGGCTCC
CCTCTGTCCA
TCTTCCTGACAAGCGCCTCTCCCCCTGCTCAGGTGCACCCACAACTCAG
AACAGGGAAG
AGCATCGTCACTCCACTAGTCTGCCTCCAGGGCTCTCTCCTTTCTAGTA
CACGGCTTGA
AGCTCCTTGAGGACACGGACCCTGGCAGTGACCTTCACAGTGCCCAGAC
CCCAAGATAA
TGCAGCCATTCATGGAACTGCAGGTTGTTCATTGGTCGCCTTTAGTTTT
CCAAAATAAG
TGTCACTTTAGCTGAAATCATTCATTAATTCAGACACCAAATCTCACAG
ATCGAAGGAG
TCAGAAATTCCTTTGAAACAACTTAGCCCAAACCTTTCTGTGTCAGTAT
GGATAAATCA
AGGCCCAATGTCTAGAAGGTCTTGGGCAAAGTTGAAATTCAGGGTCAGT
GACACAACCT
CAAGGGAGGCCCCGAAAGTGCCAGCTGCACAGCAGCCCCTGCCTGGCTT
TGCTGTTTGC
CCACCGTCCCGTGTCAGTGAATCACGGGCATCTTCAGGAGCTCAGCCTG
GGTCTTCATT
TGTTTCCCTCGGCCCCTTCCTCAGCCTCAGGACAGTGCTAGCAGCCCCC
ACACATTCTTCCCTACAGATACCATGG

Source: plasmid DC 969 (SerpinA1=A1AT) chromosome 14 NG_008290.1

### 398 bp A1AT Intron Stuffer Sequence (Downstream of Expression Cassette)

(SEQ ID NO: 19)
GCATGCAGAGTGGACAGGGGCCTCAGGGACCCCTGATCCCAGCTTTCTC
ATTGGACAGA
AGGAGGAGACTGGGGCTGGAGAGGGACCTGGGCCCCCACTAAGGCCACA
GCAGAGCCAG
GACTTTAGCTGTGCTGACTGCAGCCTGGCTTGCCTCCACTGCCCTCCTT
TGCCTCAAGA
GCAAGGGAGCCTCAGAGTGGAGGAAGCAGCCCCTGGCCTTGCCTCCCAC
CTCCCCTCCC
CTATGCTGTTTTCCTGGGACAGTGGGAGCTGGCTTAGAATGCCCTGGGG
CCCCCAGGAC
CCTGGCATTTTAACCCCTCAGGGGCAGGAAGGCAGCCTGAGATACAGAA
GAGTCCATCACCTGCTGTATGCCACACACCATCCCCACAGTCGACATTT
AAATT

Source: plasmid DC 969 (SerpinA1=A1AT) chromosome 14 NG_008290.1

### ITR-nDes-miR155-amiR-DMPK²⁰⁴-BGHpA-Stuffer-ITR (3739 bp)

(SEQ ID NO: 20)
TTGGCCACTCCCTCTCTGCGCGCTCGCTCGCTCACTGAGGCCGCCCGGGCAAAGCCCGGGCGTCG
GGCGAC
CTTTGGTCGCCCGGCCTCAGTGAGCGAGCGAGCGCGCAGAGAGGGAGTGGCCAACTCCATCACTA
GGGGTT
CCTTACGTACAATTGGGATCCTTCGAACTTGAGAGAAAACATCCCAGGGATTTACAGATCACATG
CAGGCA
GGGACCAGCTCAACCCTTCTTTAATGTCATCCAGGGAGGGGGCCAGGGATGGAGGGGAGGGGTTG
AGGAGC
GAGAGGCAGTTATTTTTGGGTGGGATTCACCACTTTTCCCATGAAGAGGGGAGACTTGGTATTTT
GTTCAA
TCATTAAGAAGACAAAGGGTTTGTTGAACTTGACCTCGGGGGGGATAGACATGGGTATGGCCTCT
AAAAAC
ATGGCCCCAGCAGCTTCAGTCCCTTTCTCGTCGATGGTCAGCACAGCCTTATGCACGGCCTGGAG
GGGAGA
GAAGCAGAGACACGTTGTAAGGCTGATCCCAGGCCTCGAGCAAGGCTCACGTGGACACCTCCCAG
GAAGCG
CTCACTCCCCCTGGACGGCCCTGGCCCTGCACATCCTCTCCCTCCCTGTCACATAGGCCTTGCTC
CTCCTC
AAGGCTTTGGCTGATGGGGCTGGCTCCCCTCTGTCCATCTTCCTGACAAGCGCCTCTCCCCCTGC
TCAGGT
GCACCCACAACTCAGAACAGGGAAGAGCATCGTCACTCCACTAGTCTGCCTCCAGGGCTCTCTCC
TTTCTA
GTACACGGCTTGAAGCTCCTTGAGGACACGGACCCTGGCAGTGACCTTCACAGTGCCCAGACCCC
AAGATA
ATGCAGCCATTCATGGAACTGCAGGTTGTTCATTGGTCGCCTTTAGTTTTCCAAAATAAGTGTCA
CTTTAG
CTGAAATCATTCATTAATTCAGACACCAAATCTCACAGATCGAAGGAGTCAGAAATTCCTTTGAA
ACAACT
TAGCCCAAACCTTTCTGTGTCAGTATGGATAAATCAAGGCCCAATGTCTAGAAGGTCTTGGGCAA
AGTTGA
AATTCAGGGTCAGTGACACAACCTCAAGGGAGGCCCCGAAAGTGCCAGCTGCACAGCAGCCCCTG
CCTGGC
TTTGCTGTTTGCCCACCGTCCCGTGTCAGTGAATCACGGGCATCTTCAGGAGCTCAGCCTGGGTC
TTCATT
TGTTTCCCTCGGCCCCTTCCTCAGCCTCAGGACAGTGCTAGCAGCCCCCACACATTCTTCCCTAC
AGATAC
CATGGCACCCATGCCTCCTCAGGTACCCCCTGCCCCCCACAGCTCCTCTCCTGTGCCTTGTTTCC
CAGCCA
TGCGTTCTCCTCTATAAATACCCGCTCTGGTATTTGGGGTTGGCAGCTGTTGCTGCCAGGGAGAT
GGTTGG
GTTGACATGCGGCTCCTGACAAAACACAAACCCCTGGTGTGTGTGGGCGTGGGTGGTGTGAGTAG
GGGGAT
GAATCAGGGAGGGGGCGGGGGACCCAGGGGGCAGGAGCCACACAAAGTCTGTGCGGGGGTGGGAG
CGCACA
TAGCAATTGGAAACTGAAAGCTTATCAGACCCTTTCTGGAAATCAGCCCACTGTTTATAAACTTG
AGGCCC
CACCCTCGAGGTACCCCCTGCCCCCCACAGCTCCTCTCCTGTGCCTTGTTTCCCAGCCATGCGTT
CTCCTC
TATAAATACCCGCTCTGGTATTTGGGGTTGGCAGCTGTTGCTGCCAGGGAGATGGTTGGGTTGAC
ATGCGG
CTCCTGACAAAACACAAACCCCTGGTGTGTGTGGGCGTGGGTGGTGTGAGTAGGGGGATGAATCA
GGGAGG
GGGCGGGGGACCCAGGGGGCAGGAGCCACACAAAGTCTGTGCGGGGGTGGGAGCGCACATAGCAA
TTGGAA
ACTGAAAGCTTCTGCAGACCTGCTTGCTGCCTGCCCTGGCGAAGGATTGGCAGGCTTGCCCGTCA
CAGGAC
CCCCGCTGGCTGACTCAGGGGCGCAGGCCTCTTGCGGGGGAGCTGGCCTCCCCGCCCCCACGGCC
ACGGGC
CGCCCTTTCCTGGCAGGACAGCGGGATCTTGCAGCTGTCAGGGGAGGGGAGGCGGGGGCTGATGT
CAGGAG
GGATACAAATAGTGCCGACGGCTGGGGGCCCTGTCTCCCCTCGCCGCATCCACTCTCCGGCCGGC
CGCCTG
CCCGCCGCCTCCTCCGTGCGCCCGCCAGCCTCGCCCGGAGCTCTGAGTAGACGAAGCTAAGGCGC
GCCTGA
GAACTTCAGGGTGAGTTTGGGGACCCTTGATTGTTCTTTCTTTTTCGCTATTGTAAAATTCATGT
TATATG
GAGGGGGCAAAGTTTTCAGGGTGTTGTTTAGAATGGGAAGATGTCCCTTGTATCACCATGCATGG
ACCCTC
ATGATAATTTTGTTTCTTTCACTTTCTACTCTGTTGACAACCATTGTCTCCTCTTATTTTCTTTT
CATTTT
CTGTAACTTTTTCGTTAAACTTTAGCTTGCATTTGTAACGAATTTTTAAATTCACTTTTGTTTAT
TTGTCA
GATTGTAAGATCCCATCGATTCGGATCCCTGGAGGCTTGCTGAAGGCTGTATGCTGAGTCGAAGA
CAGTTC
TAGGGTGTTTTGGCCACTGACTGACACCCTAGATGTCTTCGATTCAGGACACAAGGCCTGTTACT
AGCACT
CACATGGAACAAATGGCCCTCGAGCAATCAGGGTATATTATATTGTACTTCAGCACAGTTTTAGA
GAACAA
TTGTTATAATTAAATGATAAGGTAGAATATTTCTGCATATAAATTCTGGCTGGCGTGGAAATATT
CTTATT
GGTAGAAACAACTACATCCTGGTCATCATCCTGCCTTTCTCTTTATGGTTACAATGATATACACT
GTTTGA
GATGAGGATAAAATACTCTGAGTCCAAACCGGGCCCCTCTGCTAACCATGTTCATGCCTTCTTCT
TTTTCC
TACAGCTCCTGGGCAACGTGCTGACCGGTCTAGTTGCCAGCCATCTGTTGTTTGCCCCTCCCCCG
TGCCTTCCTTGACCCTGGAAGGTGCCACTCCCACTGTCCTTTCCTAATAAAATGAGGAAATTGCA
TCGCATTGTCTG
AGTAGGTGTCATTCTATTCTGGGGGGTGGGGTGGGGCAGGACAGCAAGGGGGAGGATTGGGAAGA
CAATAGCGCATGCAGAGTGGACAGGGGCCTCAGGGACCCCTGATCCCAGCTTTCTCATTGGACAG
AAGGAGGAGACTGGGGCTGGAGAGGGACCTGGGCCCCCACTAAGGCCACAGCAGAGCCAGGACTT
TAGCTGTGCTGACTGCAG
CCTGGCTTGCCTCCACTGCCCTCCTTTGCCTCAAGAGCAAGGGAGCCTCAGAGTGGAGGAAGCAG
CCCCTG
GCCTTGCCTCCCACCTCCCCTCCCCTATGCTGTTTTCCTGGGACAGTGGGAGCTGGCTTAGAATG
CCCTGG
GGCCCCCAGGACCCTGGCATTTTAACCCCTCAGGGGCAGGAAGGCAGCCTGAGATACAGAAGAGT
CCATCA
CCTGCTGTATGCCACACACCATCCCCACAGTCGACATTTAAATTAGGAACCCCTAGTGATGGAGT
TGGCCA
CTCCCTCTCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAA

TABLE 5
ITR-nDes-miR 155- amiR-DMPK²⁰⁴ Sequence Annotation
from 5′ITR to 3′ITR (3739 bp)

| Sequence | SEQ ID | |
|---|---|---|
| Numbering | NO | Annotation |
| 1-145 | 43 | 5′ ITR |
| 146-1283 | 44 | A1AT stuffer |
| 1284-2238 | 12 | nDes promoter (details below) |
| 1284-1642 | 21 | Byrne enhancer |
| 1643-1648 | n/a | Kpn1 restriction site |
| 1647-1923 | 22 | Paulin enhancer |
| 1923-1928 | n/a | HindIII restriction site |
| 1929-1934 | n/a | PstI restriction site |
| 1929-2238 | 23 | Paulin promoter |
| 2268-2578 | 45 | 5′ arm rabbit Bglobin intron |
| 2579-2584 | n/a | BamH1 restriction site |
| 2585-2611 | 46 | 5′miR155 flanking sequence |
| 2612 | n/a | additional base from Invitrogen Block-IT kit |
| 2613-2633 | 4 | DMPK²⁰⁴ miRNA guide |
| 2634-2652 | 6 | miR155 terminal loop |
| 2653-2671 | 5 | DMPK²⁰⁴ miRNA passenger |
| 2672-2674 | | additional base from Invitrogen Block-IT kit |
| 2675-2716 | 41 | 3′miR155 flanking sequence |
| 2717-2722 | n/a | Xho1 restriction site |
| 2723-3005 | 47 | 3′ arm rabbit Bglobin intron |
| 3011-3196 | 16 | minBGH poly A |
| 3197-3594 | 48 | A1AT stuffer |
| 3595-3739 | 49 | 3′ITR |

Sequence of nDes-miR 155-204 Fragment Synthesized:

(SEQ ID NO: 26)
AGATCTCCATGGCACCCATGCCTCCTCAGGTACCCCCTGCCCCCCACAGCTCCTCTCCTGTGCCT
TGTTTC
CCAGCCATGCGTTCTCCTCTATAAATACCCGCTCTGGTATTTGGGGTTGGCAGCTGTTGCTGCCA
GGGAGA
TGGTTGGGTTGACATGCGGCTCCTGACAAAACACAAACCCCTGGTGTGTGTGGGCGTGGGTGGTG
TGAGTA
GGGGGATGAATCAGGGAGGGGGCGGGGGACCCAGGGGGCAGGAGCCACACAAAGTCTGTGCGGGG
GTGGGA
GCGCACATAGCAATTGGAAACTGAAAGCTTATCAGACCCTTTCTGGAAATCAGCCCACTGTTTAT
AAACTT
GAGGCCCCACCCTCGAGGTACCCCCTGCCCCCCACAGCTCCTCTCCTGTGCCTTGTTTCCCAGCC
ATGCGT
TCTCCTCTATAAATACCCGCTCTGGTATTTGGGGTTGGCAGCTGTTGCTGCCAGGGAGATGGTTG
GGTTGA
CATGCGGCTCCTGACAAAACACAAACCCCTGGTGTGTGTGGGCGTGGGTGGTGTGAGTAGGGGGA
TGAATC
AGGGAGGGGGCGGGGGACCCAGGGGGCAGGAGCCACACAAAGTCTGTGCGGGGGTGGGAGCGCAC
ATAGCA
ATTGGAAACTGAAAGCTTCTGCAGACCTGCTTGCTGCCTGCCCTGGCGAAGGATTGGCAGGCTTG
CCCGTC
ACAGGACCCCCGCTGGCTGACTCAGGGGCGCAGGCCTCTTGCGGGGGAGCTGGCCTCCCCGCCCC
CACGGC
CACGGGCCGCCCTTTCCTGGCAGGACAGCGGGATCTTGCAGCTGTCAGGGGAGGGGAGGCGGGGG
CTGATG
TCAGGAGGGATACAAATAGTGCCGACGGCTGGGGGCCCTGTCTCCCCTCGCCGCATCCACTCTCC
GGCCGG
CCGCCTGCCCGCCGCCTCCTCCGTGCGCCCGCCAGCCTCGCCCGGAGCTCTGAGTAGACGAAGCT
AAGGCG
CGCCTGAGAACTTCAGGGTGAGTTTGGGGACCCTTGATTGTTCTTTCTTTTTCGCTATTGTAAAA
TTCATG
TTATATGGAGGGGGCAAAGTTTTCAGGGTGTTGTTTAGAATGGGAAGATGTCCCTTGTATCACCA
TGCATG
GACCCTCATGATAATTTTGTTTCTTTCACTTTCTACTCTGTTGACAACCATTGTCTCCTCTTATT
TTCTTT
TCATTTTCTGTAACTTTTTCGTTAAACTTTAGCTTGCATTTGTAACGAATTTTTAAATTCACTTT
TGTTTA
TTTGTCAGATTGTAAGATCCCATCGATTCGGATCCCTGGAGGCTTGCTGAAGGCTGTATGCTGAG
TCGAAG
ACAGTTCTAGGGTGTTTTGGCCACTGACTGACACCCTAGATGTCTTCGATTCAGGACACAAGGCC
TGTTAC
TAGCACTCACATGGAACAAATGGCCCTCGAGCAATCAGGGTATATTATATTGTACTTCAGCACAG
TTTTAG
AGAACAATTGTTATAATTAAATGATAAGGTAGAATATTTCTGCATATAAATTCTGGCTGGCGTGG
AAATAT
TCTTATTGGTAGAAACAACTACATCCTGGTCATCATCCTGCCTTTCTCTTTATGGTTACAATGAT
ATACAC
TGTTTGAGATGAGGATAAAATACTCTGAGTCCAAACCGGGCCCCTCTGCTAACCATGTTCATGCC
TTCTTC
TTTTTCCTACAGCTCCTGGGCAACGTGCTGACCGGTCTAGTTGCCAGCCATCTGTTGTTTGCCCC
TCCCCC
GTGCCTTCCTTGACCCTGGAAGGTGCCACTCCCACTGTCCTTTCCTAATAAAATGAGGAAATTGC
ATCGCA
TTGTCTGAGTAGGTGTCATTCTATTCTGGGGGGTGGGGTGGGGCAGGACAGCAAGGGGGAGGATT
GGGAAGACAATAGCGCATGCGTCGACT

nDes-miR 155-204 Promoter to polyA

(SEQ ID NO: 17)
CACCCATGCCTCCTCAGGTACCCCCTGCCCCCCACAGCTCCTCTCCTGTGCCTTGTTTC
CCAGCCATGCGTTCTCCTCTATAAATACCCGCTCTGGTATTTGGGGTTGGCAGCTGTTGCTGCCAGGGAGA
TGGTTGGGTTGACATGCGGCTCCTGACAAAACACAAACCCCTGGTGTGTGTGGGCGTGGGTGGTGTGAGTA
GGGGGATGAATCAGGGAGGGGGCGGGGGACCCAGGGGGCAGGAGCCACACAAAGTCTGTGCGGGGGTGGGA
GCGCACATAGCAATTGGAAACTGAAAGCTTATCAGACCCTTTCTGGAAATCAGCCCACTGTTTATAAACTT
GAGGCCCCACCCTCGAGGTACCCCCTGCCCCCCACAGCTCCTCTCCTGTGCCTTGTTTCCCAGCCATGCGT
TCTCCTCTATAAATACCCGCTCTGGTATTTGGGGTTGGCAGCTGTTGCTGCCAGGGAGATGGTTGGGTTGA
CATGCGGCTCCTGACAAAACACAAACCCCTGGTGTGTGTGGGCGTGGGTGGTGTGAGTAGGGGGATGAATC
AGGGAGGGGGCGGGGGACCCAGGGGGCAGGAGCCACACAAAGTCTGTGCGGGGGTGGGAGCGCACATAGCA
ATTGGAAACTGAAAGCTTCTGCAGACCTGCTTGCTGCCTGCCCTGGCGAAGGATTGGCAGGCTTGCCCGTC
ACAGGACCCCCGCTGGCTGACTCAGGGGCGCAGGCCTCTTGCGGGGGAGCTGGCCTCCCCGCCCCCACGGC
CACGGGCCGCCCTTTCCTGGCAGGACAGCGGGATCTTGCAGCTGTCAGGGGAGGGGAGGCGGGGGCTGATG
TCAGGAGGGATACAAATAGTGCCGACGGCTGGGGGCCCTGTCTCCCCTCGCCGCATCCACTCTCCGGCCGG
CCGCCTGCCCGCCGCCTCCTCCGTGCGCCCGCCAGCCTCGCCCGGAGCTCTGAGTAGACGAAGCTAAGGCG
CGCCTGAGAACTTCAGGGTGAGTTTGGGGACCCTTGATTGTTCTTTCTTTTTCGCTATTGTAAAATTCATG
TTATATGGAGGGGGCAAAGTTTTCAGGGTGTTGTTTAGAATGGGAAGATGTCCCTTGTATCACCATGCATG
GACCCTCATGATAATTTTGTTTCTTTCACTTTCTACTCTGTTGACAACCATTGTCTCCTCTTATTTTCTTT
TCATTTTCTGTAACTTTTTCGTTAAACTTTAGCTTGCATTTGTAACGAATTTTTAAATTCACTTTTGTTTA
TTTGTCAGATTGTAAGATCCCATCGATTCGGATCCCTGGAGGCTTGCTGAAGGCTGTATGCTGAGTCGAAG
ACAGTTCTAGGGTGTTTTGGCCACTGACTGACACCCTAGATGTCTTCGATTCAGGACACAAGGCCTGTTAC
TAGCACTCACATGGAACAAATGGCCCTCGAGCAATCAGGGTATATTATATTGTACTTCAGCACAGTTTTAG
AGAACAATTGTTATAATTAAATGATAAGGTAGAATATTTCTGCATATAAATTCTGGCTGGCGTGGAAATAT
TCTTATTGGTAGAAACAACTACATCCTGGTCATCATCCTGCCTTTCTCTTTATGGTTACAATGATATACAC
TGTTTGAGATGAGGATAAAATACTCTGAGTCCAAACCGGGCCCCTCTGCTAACCATGTTCATGCCTTCTTC
TTTTTCCTACAGCTCCTGGGCAACGTGCTGACCGGTCTAGTTGCCAGCCATCTGTTGTTTGCCCCTCCCCC
GTGCCTTCCTTGACCCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGGGGGAGGATTGGGAAG
ACAATAGC

### Amino Acid Sequence of AAVrh74N502I

(SEQ ID NO: 50)
MAADGYLPDWLEDNLSEGIREWWDLKPGAPKPKANQQKQDNGRGLVLPG
YKYLGPFNGLDKGEPVNAADAAALEHDKAYDQQLQAGDNPYLRYNHADA
EFQERLQEDTSFGGNLGRAVFQAKKRVLEPLGLVESPVKTAPGKKRPVE
PSPQRSPDSSTGIGKKGQQPAKKRLNFGQTGDSESVPDPQPIGEPPAGP
SGLGSGTMAAGGGAPMADNNEGADGVGSSSGNWHCDSTWLGDRVITTST
RTWALPTYNNHLYKQISNGTSGGSTNDNTYFGYSTPWGYFDFNRFHCHF
SPRDWQRLINNNWGFRPKRLNFKLFNIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQR
VSTTLSQNNNSIFAWTGATKYHLNGRDSLVNPGVAMATHKDDEERFFPS
SGVLMFGKQGAGKDNVDYSSVMLTSEEEIKTTNPVATEQYGVVADNLQQ
QNAAPIVGAVNSQGALPGMVWQNRDVYLQGPIWAKIPHTDGNFHPSPLM
GGFGLKHPPPQILIKNTPVPADPPTTFNQAKLASFITQYSTGQVSVEIE
WELQKENSKRWNPEIQYTSNYYKSTNVDFAVNTEGTYSEPRPIGTRYLT
RNL

### Nucleotide Sequence Encoding AAVrh74 N502I Capsid

(SEQ ID NO: 51)
ATGGCTGCCGATGGTTATCTTCCAGATTGGCTCGAGGACAACCTCTCTG
AGGGCATTCGCGAGTGGTGGGACCTGAAACCTGGAGCCCCGAAACCCAA
AGCCAACCAGCAAAAGCAGGACAACGGCCGGGGTCTGGTGCTTCCTGGC
TACAAGTACCTCGGACCCTTCAACGGACTCGACAAGGGGGAGCCCGTCA
ACGCGGCGGACGCAGCGGCCCTCGAGCACGACAAGGCCTACGACCAGCA
GCTCCAAGCGGGTGACAATCCGTACCTGCGGTATAATCACGCCGACGCC
GAGTTTCAGGAGCGTCTGCAAGAAGATACGTCTTTTGGGGGCAACCTCG
GGCGCGCAGTCTTCCAGGCCAAAAAGCGGGTTCTCGAACCTCTGGGCCT
GGTTGAATCGCCGGTTAAGACGGCTCCTGGAAAGAAGAGACCGGTAGAG
CCATCACCCCAGCGCTCTCCAGACTCCTCTACGGGCATCGGCAAGAAAG
GCCAGCAGCCCGCAAAAAAGAGACTCAATTTTGGGCAGACTGGCGACTC
AGAGTCAGTCCCCGACCCTCAACCAATCGGAGAACCACCAGCAGGCCCC
TCTGGTCTGGGATCTGGTACAATGGCTGCAGGCGGTGGCGCTCCAATGG
CAGACAATAACGAAGGCGCCGACGGAGTGGGTAGTTCCTCAGGAAATTG
GCATTGCGATTCCACATGGCTGGGCGACAGAGTCATCACCACCAGCACC
CGCACCTGGGCCCTGCCCACCTACAACAACCACCTCTACAAGCAAATCT
CCAACGGGACCTCGGGAGGAAGCACCAACGACAACACCTACTTCGGCTA
CAGCACCCCCTGGGGGTATTTTGACTTCAACAGATTCCACTGCCACTTT
TCACCACGTGACTGGCAGCGACTCATCAACAACAACTGGGGATTCCGGC
CCAAGAGGCTCAACTTCAAGCTCTTCAACATCCAAGTCAAGGAGGTCAC
GCAGAATGAAGGCACCAAGACCATCGCCAATAACCTTACCAGCACGATT
CAGGTCTTTACGGACTCGGAATACCAGCTCCCGTACGTGCTCGGCTCGG
CGCACCAGGGCTGCCTGCCTCCGTTCCCGGCGGACGTCTTCATGATTCC
TCAGTACGGGTACCTGACTCTGAACAATGGCAGTCAGGCTGTGGGCCGG
TCGTCCTTCTACTGCCTGGAGTACTTTCCTTCTCAAATGCTGAGAACGG
GCAACAACTTTGAATTCAGCTACAACTTCGAGGACGTGCCCTTCCACAG
CAGCTACGCGCACAGCCAGAGCCTGGACCGGCTGATGAACCCTCTCATC
GACCAGTACTTGTACTACCTGTCCCGGACTCAAAGCACGGGCGGTACTG
CAGGAACTCAGCAGTTGCTATTTTCTCAGGCCGGGCCTAACAACATGTC
GGCTCAGGCCAAGAACTGGCTACCCGGTCCCTGCTACCGGCAGCAACGC
GTCTCCACGACACTGTCGCAGAACAACAACAGCATCTTTGCCTGGACGG
GTGCCACCAAGTATCATCTGAATGGCAGAGACTCTCTGGTGAATCCTGG
CGTTGCCATGGCTACCCACAAGGACGACGAAGAGCGATTTTTTCCATCC
AGCGGAGTCTTAATGTTTGGGAAACAGGGAGCTGGAAAAGACAACGTGG
ACTATAGCAGCGTGATGCTAACCAGCGAGGAAGAAATAAAGACCACCAA
CCCAGTGGCCACAGAACAGTACGGCGTGGTGGCCGATAACCTGCAACAG
CAAAACGCCGCTCCTATTGTAGGGGCCGTCAATAGTCAAGGAGCCTTAC
CTGGCATGGTGTGGCAGAACCGGGACGTGTACCTGCAGGGTCCCATCTG
GGCCAAGATTCCTCATACGGACGGCAACTTTCATCCCTCGCCGCTGATG
GGAGGCTTTGGACTGAAGCATCCGCCTCCTCAGATCCTGATTAAAAACA
CACCTGTTCCCGCGGATCCTCCGACCACCTTCAATCAGGCCAAGCTGGC
TTCTTTCATCACGCAGTACAGTACCGGCCAGGTCAGCGTGGAGATCGAG
TGGGAGCTGCAGAAGGAGAACAGCAAACGCTGGAACCCAGAGATTCAGT
ACACTTCCAACTACTACAAATCTACAAATGTGGACTTTGCTGTCAATAC
TGAGGGTACTTATTCCGAGCCTCGCCCCATTGGCACCCGTTACCTCACC
CGTAATCTGTAA

### Amino Acid Sequence of AAVrh74W505R

(SEQ ID NO: 52)
MAADGYLPDWLEDNLSEGIREWWDLKPGAPKPKANQQKQDNGRGLVLPG
YKYLGPFNGLDKGEPVNAADAAALEHDKAYDQQLQAGDNPYLRYNHADA
EFQERLQEDTSFGGNLGRAVFQAKKRVLEPLGLVESPVKTAPGKKRPVE
PSPQRSPDSSTGIGKKGQQPAKKRLNFGQTGDSESVPDPQPIGEPPAGP
SGLGSGTMAAGGGAPMADNNEGADGVGSSSGNWHCDSTWLGDRVITTST
RTWALPTYNNHLYKQISNGTSGGSTNDNTYFGYSTPWGYFDFNRFHCHF
SPRDWQRLINNNWGFRPKRLNFKLFNIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQR
VSTTLSQNNNSNFARTGATKYHLNGRDSLVNPGVAMATHKDDEERFFPS
SGVLMFGKQGAGKDNVDYSSVMLTSEEEIKTTNPVATEQYGVVADNLQQ
QNAAPIVGAVNSQGALPGMVWQNRDVYLQGPIWAKIPHTDGNFHPSPLM
GGFGLKHPPPQILIKNTPVPADPPTTFNQAKLASFITQYSTGQVSVEIE
WELQKENSKRWNPEIQYTSNYYKSTNVDFAVNTEGTYSEPRPIGTRYLT
RNL

### Nucleotide Sequence Encoding AAVrh74W505R

(SEQ ID NO: 53)
ATGGCTGCCGATGGTTATCTTCCAGATTGGCTCGAGGACAACCTCTCTG
AGGGCATTCGCGAGTGGTGGGACCTGAAACCTGGAGCCCCGAAACCCAA
AGCCAACCAGCAAAAGCAGGACAACGGCCGGGGTCTGGTGCTTCCTGGC
TACAAGTACCTCGGACCCTTCAACGGACTCGACAAGGGGGAGCCCGTCA
ACGCGGCGGACGCAGCGGCCCTCGAGCACGACAAGGCCTACGACCAGCA
GCTCCAAGCGGGTGACAATCCGTACCTGCGGTATAATCACGCCGACGCC
GAGTTTCAGGAGCGTCTGCAAGAAGATACGTCTTTTGGGGGCAACCTCG
GGCGCGCAGTCTTCCAGGCCAAAAAGCGGGTTCTCGAACCTCTGGGCCT
GGTTGAATCGCCGGTTAAGACGGCTCCTGGAAAGAAGAGACCGGTAGAG
CCATCACCCCAGCGCTCTCCAGACTCCTCTACGGGCATCGGCAAGAAAG
GCCAGCAGCCCGCAAAAAAGAGACTCAATTTTGGGCAGACTGGCGACTC
AGAGTCAGTCCCCGACCCTCAACCAATCGGAGAACCACCAGCAGGCCCC
TCTGGTCTGGGATCTGGTACAATGGCTGCAGGCGGTGGCGCTCCAATGG
CAGACAATAACGAAGGCGCCGACGGAGTGGGTAGTTCCTCAGGAAATTG
GCATTGCGATTCCACATGGCTGGGCGACAGAGTCATCACCACCAGCACC
CGCACCTGGGCCCTGCCCACCTACAACAACCACCTCTACAAGCAAATCT
CCAACGGGACCTCGGGAGGAAGCACCAACGACAACACCTACTTCGGCTA
CAGCACCCCCTGGGGGTATTTTGACTTCAACAGATTCCACTGCCACTTT
TCACCACGTGACTGGCAGCGACTCATCAACAACAACTGGGGATTCCGGC
CCAAGAGGCTCAACTTCAAGCTCTTCAACATCCAAGTCAAGGAGGTCAC
GCAGAATGAAGGCACCAAGACCATCGCCAATAACCTTACCAGCACGATT
CAGGTCTTTACGGACTCGGAATACCAGCTCCCGTACGTGCTCGGCTCGG
CGCACCAGGGCTGCCTGCCTCCGTTCCCGGCGGACGTCTTCATGATTCC
TCAGTACGGGTACCTGACTCTGAACAATGGCAGTCAGGCTGTGGGCCGG
TCGTCCTTCTACTGCCTGGAGTACTTTCCTTCTCAAATGCTGAGAACGG
GCAACAACTTTGAATTCAGCTACAACTTCGAGGACGTGCCCTTCCACAG
CAGCTACGCGCACAGCCAGAGCCTGGACCGGCTGATGAACCCTCTCATC
GACCAGTACTTGTACTACCTGTCCCGGACTCAAAGCACGGGCGGTACTG
CAGGAACTCAGCAGTTGCTATTTTCTCAGGCCGGGCCTAACAACATGTC
GGCTCAGGCCAAGAACTGGCTACCCGGTCCCTGCTACCGGCAGCAACGC
GTCTCCACGACACTGTCGCAGAACAACAACAGCAACTTTGCCAGGACGG
GTGCCACCAAGTATCATCTGAATGGCAGAGACTCTCTGGTGAATCCTGG
CGTTGCCATGGCTACCCACAAGGACGACGAAGAGCGATTTTTTCCATCC
AGCGGAGTCTTAATGTTTGGGAAACAGGGAGCTGGAAAAGACAACGTGG
ACTATAGCAGCGTGATGCTAACCAGCGAGGAAGAAATAAAGACCACCAA
CCCAGTGGCCACAGAACAGTACGGCGTGGTGGCCGATAACCTGCAACAG
CAAAACGCCGCTCCTATTGTAGGGGCCGTCAATAGTCAAGGAGCCTTAC
CTGGCATGGTGTGGCAGAACCGGGACGTGTACCTGCAGGGTCCCATCTG
GGCCAAGATTCCTCATACGGACGGCAACTTTCATCCCTCGCCGCTGATG
GGAGGCTTTGGACTGAAGCATCCGCCTCCTCAGATCCTGATTAAAAACA
CACCTGTTCCCGCGGATCCTCCGACCACCTTCAATCAGGCCAAGCTGGC
TTCTTTCATCACGCAGTACAGTACCGGCCAGGTCAGCGTGGAGATCGAG
TGGGAGCTGCAGAAGGAGAACAGCAAACGCTGGAACCCAGAGATTCAGT
ACACTTCCAACTACTACAAATCTACAAATGTGGACTTTGCTGTCAATAC
TGAGGGTACTTATTCCGAGCCTCGCCCCATTGGCACCCGTTACCTCACC
CGTAATCTGTAA

1. A recombinant adeno-associated virus (rAAV) particle comprising:
a) an RNAi comprising a first strand and a second strand, wherein:
i) the first strand and the second strand form a duplex;
ii) the first strand comprises a guide region, wherein the guide region comprises nucleic acid with the sequence 5′-AGUCGAAGACAGUUCUAGGGU-3′ (SEQ ID NO:1) or with a sequence with about 90% identity to the sequence of SEQ ID NO:1; and
iii) the second strand comprises a non-guide region; and
b) an AAV capsid comprising an amino acid sequence with about 90% identity to a wildtype AAVrh74 capsid.

2. The rAAV particle of claim 1, wherein:
the non-guide region comprises nucleic acid with the sequence 5′-ACCCUAGAUGUCUUCGAUU-3′ (SEQ ID NO:2) or a with a sequence with about 90% identity to the sequence of SEQ ID NO:2;
the RNAi is a small inhibitory RNA (siRNA), a microRNA (miRNA), or a small hairpin RNA (shRNA); and/or
the first strand and the second strand are linked by means of an RNA linker capable of forming a loop structure, optionally wherein:
the RNA linker comprises from about 4 to about 50 nucleotides;
the loop structure comprises nucleic sequence with of SEQ ID NO:3 or with a sequence with about 90% identity to the sequence of SEQ ID NO:3; and/or
the RNAi comprises 5′ to 3′ the second strand, the RNA linker, and the first strand or 5′ to 3′ the first strand, the RNA linker, and the second strand.

3-9. (canceled)

10. The rAAV particle of claim 1, wherein the RNAi comprises nucleic acid with the sequence of SEQ ID NO:7 or with a sequence with about 90% identity to the sequence of SEQ ID NO:7.

11. (canceled)

12. The rAAV particle of claim 1, wherein the RNAi further comprises a scaffold, optionally wherein:
the scaffold comprises all or a portion of the nucleic acid of SEQ ID No: 11;
the scaffold has a 5′ arm, wherein the 5′ arm is located 5′ to the nucleic acid encoding the RNAi, and a 3′ arm, wherein the 3′ arm is located 3′ to the nucleic acid encoding the RNAi; and/or
the scaffold is a miR-155 scaffold, optionally wherein the miR-155 scaffold comprises the nucleic acid of SEQ ID NO:9 or a sequence with about 90% identity to the sequence of SEQ ID NO: 9 located 5′ to the RNAi and/or the miR-155 scaffold comprises the nucleic acid of SEQ ID NO: 10 or a sequence with about 90% identity to the sequence of SEQ ID NO: 10 located 3′ to the RNAi.

13-18. (canceled)

19. The rAAV particle of claim 1, wherein the RNAi targets RNA encoding a polypeptide associated with myotonic dystrophy-1 (DM1), optionally wherein the polypeptide is dystrophia myotonica protein kinase (DMPK).

20-43. (canceled)

44. An expression cassette, comprising a nucleic acid encoding a transgene and a modified desmin promoter, wherein the modified desmin promoter comprises one or more enhancer elements and the promoter for the human desmin gene.

45. The expression cassette of claim 44, wherein:
the modified desmin promoter comprises two enhancer elements and the promoter for the human desmin gene;
the modified desmin promoter comprises one or more Byrne enhancer elements and/or one or more Paulin enhancer elements;
the modified desmin promoter comprises one or more enhancer elements comprising the nucleotide sequence of SEQ ID NO:21 or a nucleotide sequence with about 90% identity to the sequence of SEQ ID NO:21 and/or one or more enhancer elements comprising the nucleotide sequence of SEQ ID NO:22 or a nucleotide sequence with about 90% identity to the sequence of SEQ ID NO:22; and/or
the desmin promoter comprises the nucleotide sequence of SEQ ID NO: 12 or a sequence with about 90% identity to the nucleotide sequence of SEQ ID NO:12.

46-48. (canceled)

49. The expression cassette of claim 44, wherein the expression cassette further comprises an intron, optionally wherein:
the intron is a rabbit β-globin intron;
the intron comprises the nucleotide sequence of SEQ ID NO:13 or a sequence with about 90% identity to the sequence of SEQ ID NO: 13; and/or
the nucleic acid encoding the transgene is embedded in the intron.

50-52. (canceled)

53. The expression cassette of claim 49, wherein the intron comprises a 5′ arm and a 3′ arm, wherein the 5′ arm is located 5′ to the nucleic acid encoding the transgene and the 3′ arm is located 3′ to the nucleic acid encoding the transgene, optionally wherein the 5′ arm of the intron comprises the nucleotide sequence of SEQ ID NO:14 or a sequence with about 90% identity to the sequence of SEQ ID NO: 14 and/or the 3′ arm of the intron comprises the nucleotide sequence of SEQ ID NO: 15 or a sequence with about 90% identity to the sequence of SEQ ID NO:15.

54-61. (canceled)

62. A vector comprising the expression cassette of claim 44.

63. The vector of claim 62, wherein the expression cassette is flanked by one or more stuffer nucleic acid sequences, optionally wherein:
the one or more stuffer nucleic acid sequences is derived from the human SerpinA1 gene;
a stuffer nucleic acid sequence located 5′ to the expression cassette is derived from the human SerpinA1 gene;
a stuffer sequence located 5′ to the expression cassette comprises the nucleotide sequence of SEQ ID NO: 18 or a sequence with about 90% identity to the sequence of SEQ ID NO:18;
a stuffer nucleic acid sequence located 3′ to the expression cassette is derived from the human SerpinA1 gene; and/or
a stuffer sequence located 3′ to the expression cassette comprises the nucleotide sequence of SEQ ID NO: 19 or a sequence with about 90% identity to the sequence of SEQ ID NO:19.

64-68. (canceled)

69. The vector of claim 62, wherein the vector is a recombinant adeno-associated virus (rAAV) vector, optionally wherein:
the expression cassette is flanked by one or more AAV inverted terminal repeat (ITR) sequences;
the expression cassette is flanked by two AAV ITRs; and/or
the AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype ITRs.

70-73. (canceled)

74. The rAAV vector of claim 69, wherein the rAAV vector comprises the nucleotide sequence of SEQ ID NO:20 or a sequence with about 90% identity to the sequence of SEQ ID NO:20.

75-76. (canceled)

77. The rAAV particle of claim 1, wherein the AAV capsid comprises an amino acid sequence comprising an amino acid substitution at position 502, optionally wherein:
the amino acid substitution at position 502 is an isoleucine (I);
the rAAV particle comprises a AAVrh74 N502I serotype capsid; and/or
the ITR is an AAV2 ITR and the capsid of the rAAV particle is an AAVrh74 N502I serotype capsid.

78-80. (canceled)

81. The rAAV particle of claim 1, wherein the AAV capsid comprises an amino acid sequence comprising an amino acid substitution at position 505, optionally wherein:
the amino acid substitution at position 505 is an arginine (R);
the rAAV particle comprises a AAVrh74 W505R serotype capsid; and/or
the ITR is an AAV2 ITR and the capsid of the rAAV particle is an AAVrh74 W505R serotype capsid.

82-84. (canceled)

85. An rAAV particle comprising an rAAV vector and a capsid, wherein the rAAV vector comprises the following nucleic acids 5′ to 3′,
an AAV2 ITR,
nucleic acid encoding a stuffer nucleic acid sequence from the human serpinA1 gene,
a Byrne desmin enhancer element,
a Paulin desmin enhancer element,
a desmin promoter,
a 5′ arm of a rabbit β-globin intron,
a 5′ miR155 scaffold sequence,
a DMPK²⁰⁴ miRNA guide sequence,
a miR155 terminal loop sequence,
a DMPK²⁰⁴ miRNA passenger sequence,
a 3′ miR155 scaffold sequence,
a 3′ arm of a rabbit β-globin intron,
a minimal bovine growth hormone polyadenylation sequence,
nucleic acid encoding a stuffer nucleic acid sequence from the human serpinA1 gene, and
an AAV2 ITR;
and wherein the capsid is an AAVrh74 N502I capsid.

86. The rAAV particle of claim 85, wherein the rAAV vector comprises the following nucleic acids 5′ to 3′,
an AAV2 ITR comprising the polynucleotide sequence of SEQ ID NO:43,
nucleic acid encoding a stuffer nucleic acid sequence from the human serpinA1 gene comprising the polynucleotide sequence of SEQ ID NO:18,
a Byrne desmin enhancer element comprising the polynucleotide sequence of SEQ ID NO: 21,
a Paulin desmin enhancer element comprising the polynucleotide sequence of SEQ ID NO: 22,
a desmin promoter comprising the polynucleotide sequence of SEQ ID NO:23,
a 5′ arm of a rabbit β-globin intron comprising the polynucleotide sequence of SEQ ID NO: 14,
a 5′ miR155 scaffold sequence comprising the polynucleotide sequence of SEQ ID NO: 40,
a DMPK²⁰⁴ miRNA guide sequence comprising the polynucleotide sequence of SEQ ID NO: 4,
a miR155 terminal loop sequence comprising the polynucleotide sequence of SEQ ID NO: 6,
a DMPK²⁰⁴ miRNA passenger sequence comprising the polynucleotide sequence of SEQ ID NO: 5,
a 3′ miR155 scaffold sequence comprising the polynucleotide sequence of SEQ ID NO: 41,
a 3′ arm of a rabbit β-globin intron comprising the polynucleotide sequence of SEQ ID NO: 15,
a minimal bovine growth hormone polyadenylation sequence comprising the polynucleotide sequence of SEQ ID NO:16,
nucleic acid encoding a stuffer nucleic acid sequence from the human serpinA1 gene comprising the polynucleotide sequence of SEQ ID NO:19, and
an AAV2 ITR comprising the polynucleotide sequence of SEQ ID NO:49;
and wherein the capsid is an AAVrh74 N502I capsid.

87. The rAAV particle of claim 85, wherein the AAVrh74 N502I capsid comprises capsid proteins comprising the amino acid sequence of SEQ ID NO: 50.

88. An rAAV particle comprising an rAAV vector and a capsid, wherein the rAAV vector comprises the following nucleic acids 5′ to 3′, an AAV2 ITR, nucleic acid encoding a stuffer nucleic acid sequence from the human serpinA1 gene, a Byrne desmin enhancer element, a Paulin desmin enhancer element, a desmin promoter, a 5′ arm of a rabbit β-globin intron, a 5′ miR155 scaffold sequence, a DMPK²⁰⁴ miRNA guide sequence, a miR155 terminal loop sequence, a DMPK²⁰⁴ miRNA passenger sequence, a 3′ miR155 scaffold sequence, a 3′ arm of a rabbit β-globin intron, a minimal bovine growth hormone polyadenylation sequence, nucleic acid encoding a stuffer nucleic acid sequence from the human serpinA1 gene, and an AAV2 ITR; and wherein the capsid is an AAVrh74 W505R capsid.

89. The rAAV particle of claim 88, wherein the rAAV vector comprises the following nucleic acids 5′ to 3′, an AAV2 ITR comprising the polynucleotide sequence of SEQ ID NO:43, nucleic acid encoding a stuffer nucleic acid sequence from the human serpinA1 gene comprising the polynucleotide sequence of SEQ ID NO:18, a Byrne desmin enhancer element comprising the polynucleotide sequence of SEQ ID NO:21, a Paulin desmin enhancer element comprising the polynucleotide sequence of SEQ ID NO:22, a desmin promoter comprising the polynucleotide sequence of SEQ ID NO:23, a 5′ arm of a rabbit β-globin intron comprising the polynucleotide sequence of SEQ ID NO: 14, a 5′ miR155 scaffold sequence comprising the polynucleotide sequence of SEQ ID NO:40, a DMPK²⁰⁴ miRNA guide sequence comprising the polynucleotide sequence of SEQ ID NO:4, a miR155 terminal loop sequence comprising the polynucleotide sequence of SEQ ID NO:6, a DMPK²⁰⁴ miRNA passenger sequence comprising the polynucleotide sequence of SEQ ID NO:5, a 3′ miR155 scaffold sequence comprising the polynucleotide sequence of SEQ ID NO:41, a 3′ arm of a rabbit β-globin intron comprising the polynucleotide sequence of SEQ ID NO:15, a minimal bovine growth hormone polyadenylation sequence comprising the polynucleotide sequence of SEQ ID NO: 16, nucleic acid encoding a stuffer nucleic acid sequence from the human serpinA1 gene comprising the polynucleotide sequence of SEQ ID NO: 19, and an AAV2 ITR comprising the polynucleotide sequence of SEQ ID NO:49; and wherein the capsid is an AAVrh74 W505R capsid.

90. The rAAV particle of claim 88, wherein the AAVrh74 W505R capsid comprises capsid proteins comprising the amino acid sequence of SEQ ID NO: 52.

91. (canceled)

92. A pharmaceutical composition comprising the rAAV particle of claim 1.

93. (canceled)

94. A kit comprising the rAAV particle of claim 1.

95-97. (canceled)

98. A method for treating myotonic dystrophy-1 (DM1) in a mammal in need thereof comprising administering to the mammal an effective amount of the rAAV particle of claim 1.

99-100. (canceled)

101. A method for treating myotonic dystrophy-1 (DM1) in a mammal in need thereof comprising administering to the mammal an effective amount of the rAAV particle of claim 88.

102-103. (canceled)

104. The method of claim 98, wherein the effective amount of the rAAV particle is a dose of about 1×10⁸ to about 2×10¹³ genome copies/mL and/or the effective amount of the rAAV particle is a dose of about 1×10⁸ to about 2×10¹⁴ genome copies/kg of body weight.

105-114. (canceled)

115. The method of claim 98, wherein the RNAi is administered in combination with an immunosuppressive agent, wherein the immunosuppressive agent is administered before, at the same time, and/or after administration of the RNAi.

116-117. (canceled)

118. The method of claim 98, wherein the splicing of gene transcripts is measured after administration of the rAAV particle.

119-120. (canceled)

121. The method of claim 118, wherein the gene transcripts measured comprise one or more of genes selected from MBNL1, SOS1, PKM, zTTN, GOGLA4, CLASP1, LDB3, MBNL2, SPAG9, DNAseI, TNNT3 and ZBTB49.

122-125. (canceled)

126. The expression cassette of claim 44, wherein the nucleic acid encoding the transgene comprise any one or more of:
a 5′ miR155 scaffold sequence comprising the polynucleotide sequence of SEQ ID NO: 40,
a DMPK²⁰⁴ miRNA guide sequence comprising the polynucleotide sequence of SEQ ID NO: 4,
a miR155 terminal loop sequence comprising the polynucleotide sequence of SEQ ID NO: 6,
a DMPK²⁰⁴ miRNA passenger sequence comprising the polynucleotide sequence of SEQ ID NO: 5, and
a 3′ miR155 scaffold sequence comprising the polynucleotide sequence of SEQ ID NO: 41.


SEQUENCE LISTING (53 sequences)

SEQ ID NO: 1 - RNA, 21 nt, synthetic construct
  misc_feature 1..21: note = Synthetic Construct
  source 1..21: mol_type = other RNA
agtcgaagacagttctagggt

SEQ ID NO: 2 - RNA, 19 nt, synthetic construct
  misc_feature 1..19: note = Synthetic Construct
  source 1..19: mol_type = other RNA
accctagatgtcttcgatt

SEQ ID NO: 3 - RNA, 19 nt, synthetic construct
  misc_feature 1..19: note = Synthetic Construct
  source 1..19: mol_type = other RNA
gttttggccactgactgac

SEQ ID NO: 4 - DNA, 21 bp, synthetic construct
  misc_feature 1..21: note = Synthetic Construct
  source 1..21: mol_type = other DNA
agtcgaagacagttctagggt

SEQ ID NO: 5 - DNA, 19 bp, synthetic construct
  misc_feature 1..19: note = Synthetic Construct
  source 1..19: mol_type = other DNA
accctagatgtcttcgatt

SEQ ID NO: 6 - DNA, 19 bp, synthetic construct
  misc_feature 1..19: note = Synthetic Construct
  source 1..19: mol_type = other DNA
gttttggccactgactgac

SEQ ID NO: 7 - RNA, 60 nt, synthetic construct
  misc_feature 1..60: note = Synthetic Construct
  source 1..60: mol_type = other RNA
agtcgaagacagttctagggttgttttggccactgactgacaccctagatgtcttcgatt

SEQ ID NO: 8 - DNA, 60 bp, synthetic construct
  misc_feature 1..60: note = Synthetic Construct
  source 1..60: mol_type = other DNA
agtcgaagacagttctagggttgttttggccactgactgacaccctagatgtcttcgatt

SEQ ID NO: 9 - RNA, 27 nt, synthetic construct
  misc_feature 1..27: note = Synthetic Construct
  source 1..27: mol_type = other RNA
ctggaggcttgctgaaggctgtatgct

SEQ ID NO: 10 - RNA, 42 nt, synthetic construct
  misc_feature 1..42: note = Synthetic Construct
  source 1..42: mol_type = other RNA
gacacaaggcctgttactagcactcacatggaacaaatggcc

SEQ ID NO: 11 - RNA, 69 nt, synthetic construct
  misc_feature 1..69: note = Synthetic Construct
  source 1..69: mol_type = other RNA
ctggaggcttgctgaaggctgtatgctgacacaaggcctgttactagcactcacatggaacaaatggcc

SEQ ID NO: 12 - DNA, 955 bp, synthetic construct
  misc_feature 1..955: note = Synthetic Construct
  source 1..955: mol_type = other DNA
cacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttctgcagacctgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccg

SEQ ID NO: 13 - DNA, 561 bp, Oryctolagus cuniculus
  source 1..561: mol_type = other DNA
gtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattccaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacag

SEQ ID NO: 14 - DNA, 296 bp, synthetic construct
  misc_feature 1..296: note = Synthetic Construct
  source 1..296: mol_type = other DNA
gtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattc

SEQ ID NO: 15 - DNA, 265 bp, synthetic construct
  misc_feature 1..265: note = Synthetic Construct
  source 1..265: mol_type = other DNA
caatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacag

SEQ ID NO: 16 - DNA, 186 bp, synthetic construct
  misc_feature 1..186: note = Synthetic Construct
  source 1..186: mol_type = other DNA
tctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagc

SEQ ID NO: 17 - DNA, 1913 bp, synthetic construct
  misc_feature 1..1913: note = Synthetic Construct
  source 1..1913: mol_type = other DNA
cacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttctgcagacctgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccggagctctgagtagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcggatccctggaggcttgctgaaggctgtatgctgagtcgaagacagttctagggtgttttggccactgactgacaccctagatgtcttcgattcaggacacaaggcctgttactagcactcacatggaacaaatggccctcgagcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctgaccggtctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagc

SEQ ID NO: 18 - DNA, 1138 bp, synthetic construct
  misc_feature 1..1138: note = Synthetic Construct
  source 1..1138: mol_type = other DNA
tacgtacaattgggatccttcgaacttgagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccactagtctgcctccagggctctctcctttctagtacacggcttgaagctccttgaggacacggaccctggcagtgaccttcacagtgcccagaccccaagataatgcagccattcatggaactgcaggttgttcattggtcgcctttagttttccaaaataagtgtcactttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagcccctgcctggctttgctgtttgcccaccgtcccgtgtcagtgaatcacgggcatcttcaggagctcagcctgggtcttcatttgtttccctcggccccttcctcagcctcaggacagtgctagcagcccccacacattcttccctacagataccatgg

SEQ ID NO: 19 - DNA, 398 bp, synthetic construct
  misc_feature 1..398: note = Synthetic Construct
  source 1..398: mol_type = other DNA
gcatgcagagtggacaggggcctcagggacccctgatcccagctttctcattggacagaaggaggagactggggctggagagggacctgggcccccactaaggccacagcagagccaggactttagctgtgctgactgcagcctggcttgcctccactgccctcctttgcctcaagagcaagggagcctcagagtggaggaagcagcccctggccttgcctcccacctcccctcccctatgctgttttcctgggacagtgggagctggcttagaatgccctggggcccccaggaccctggcattttaacccctcaggggcaggaaggcagcctgagatacagaagagtccatcacctgctgtatgccacacaccatccccacagtcgacatttaaatt

SEQ ID NO: 20 - DNA, 3739 bp, synthetic construct
  misc_feature 1..3739: note = Synthetic Construct
  source 1..3739: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatccttcgaacttgagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccactagtctgcctccagggctctctcctttctagtacacggcttgaagctccttgaggacacggaccctggcagtgaccttcacagtgcccagaccccaagataatgcagccattcatggaactgcaggttgttcattggtcgcctttagttttccaaaataagtgtcactttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagcccctgcctggctttgctgtttgcccaccgtcccgtgtcagtgaatcacgggcatcttcaggagctcagcctgggtcttcatttgtttccctcggccccttcctcagcctcaggacagtgctagcagcccccacacattcttccctacagataccatggcacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttctgcagacctgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccggagctctgagtagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcggatccctggaggcttgctgaaggctgtatgctgagtcgaagacagttctagggtgttttggccactgactgacaccctagatgtcttcgattcaggacacaaggcctgttactagcactcacatggaacaaatggccctcgagcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctgaccggtctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcgcatgcagagtggacaggggcctcagggacccctgatcccagctttctcattggacagaaggaggagactggggctggagagggacctgggcccccactaaggccacagcagagccaggactttagctgtgctgactgcagcctggcttgcctccactgccctcctttgcctcaagagcaagggagcctcagagtggaggaagcagcccctggccttgcctcccacctcccctcccctatgctgttttcctgggacagtgggagctggcttagaatgccctggggcccccaggaccctggcattttaacccctcaggggcaggaaggcagcctgagatacagaagagtccatcacctgctgtatgccacacaccatccccacagtcgacatttaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 21 - DNA, 359 bp, synthetic construct
  misc_feature 1..359: note = Synthetic Construct
  source 1..359: mol_type = other DNA
cacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcga

SEQ ID NO: 22 - DNA, 277 bp, synthetic construct
  misc_feature 1..277: note = Synthetic Construct
  source 1..277: mol_type = other DNA
ccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaa

SEQ ID NO: 23 - DNA, 310 bp, synthetic construct
  misc_feature 1..310: note = Synthetic Construct
  source 1..310: mol_type = other DNA
ctgcagacctgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccg

SEQ ID NO: 24 - RNA, 132 nt, synthetic construct
  misc_feature 1..132: note = Synthetic Construct
  source 1..132: mol_type = other RNA
ctggaggcttgctgaaggctgtatgctgagtcgaagacagttctagggtgttttggccactgactgacaccctagatgtcttcgattcaggacacaaggcctgttactagcactcacatggaacaaatggcc

SEQ ID NO: 25 - DNA, 132 bp, synthetic construct
  misc_feature 1..132: note = Synthetic Construct
  source 1..132: mol_type = other DNA
ctggaggcttgctgaaggctgtatgctgagtcgaagacagttctagggtgttttggccactgactgacaccctagatgtcttcgattcaggacacaaggcctgttactagcactcacatggaacaaatggcc

SEQ ID NO: 26 - DNA, 1938 bp, synthetic construct
  misc_feature 1..1938: note = Synthetic Construct
  source 1..1938: mol_type = other DNA
agatctccatggcacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttctgcagacctgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccggagctctgagtagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcggatccctggaggcttgctgaaggctgtatgctgagtcgaagacagttctagggtgttttggccactgactgacaccctagatgtcttcgattcaggacacaaggcctgttactagcactcacatggaacaaatggccctcgagcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctgaccggtctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcgcatgcgtcgact

SEQ ID NO: 27 - DNA, 113 bp, synthetic construct
  misc_feature 1..113: note = Synthetic Construct
  source 1..113: mol_type = other DNA
ccactccctctctgcgcgctcgctcgctcactgaggccgggcgaccaaaggtcgcccgacgcccgggctttgcccgggcggcctcagtgagcgagcgagcgcgcagagaggga

SEQ ID NO: 28 - DNA, 21 bp, Homo sapiens
  source 1..21: mol_type = other DNA
accctagaactgtcttcgact

SEQ ID NO: 29 - DNA, 21 bp, Macaca mulatta
  source 1..21: mol_type = other DNA
accctagaactgtcttcgact

SEQ ID NO: 30 - DNA, 21 bp, Mus musculus
  source 1..21: mol_type = other DNA
accctaagactccaagccatc

SEQ ID NO: 31 - DNA, 21 bp, Rattus rattus
  source 1..21: mol_type = other DNA
accctaagactccaagccatt

SEQ ID NO: 32 - DNA, 21 bp, Canis lupus
  source 1..21: mol_type = other DNA
accctagagccccaagccgga

SEQ ID NO: 33 - DNA, 23 bp, synthetic construct
  misc_feature 1..23: note = Synthetic Construct
  source 1..23: mol_type = other DNA
agtcgaagacagttctagggtgt

SEQ ID NO: 34 - DNA, 24 bp, synthetic construct
  misc_feature 1..24: note = Synthetic Construct
  source 1..24: mol_type = other DNA
agtcgaagacagttctagggtgtt

SEQ ID NO: 35 - DNA, 25 bp, synthetic construct
  misc_feature 1..25: note = Synthetic Construct
  source 1..25: mol_type = other DNA
agtcgaagacagttctagggtgttt

SEQ ID NO: 36 - DNA, 21 bp, synthetic construct
  misc_feature 1..21: note = Synthetic Construct
  source 1..21: mol_type = other DNA
agtcgaagacagttctagggt

SEQ ID NO: 37 - DNA, 26 bp, synthetic construct
  misc_feature 1..26: note = Synthetic Construct
  source 1..26: mol_type = other DNA
agtcgaagacagttctagggtgtttt

SEQ ID NO: 38 - DNA, 22 bp, synthetic construct
  misc_feature 1..22: note = Synthetic Construct
  source 1..22: mol_type = other DNA
agtcgaagacagttctagggtg

SEQ ID NO: 39 - protein, 738 aa, synthetic construct
  REGION 1..738: note = Synthetic Construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNYIGSRGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 40 - DNA, 27 bp, synthetic construct
  misc_feature 1..27: note = Synthetic Construct
  source 1..27: mol_type = other DNA
ctggaggcttgctgaaggctgtatgct

SEQ ID NO: 41 - DNA, 42 bp, synthetic construct
  misc_feature 1..42: note = Synthetic Construct
  source 1..42: mol_type = other DNA
gacacaaggcctgttactagcactcacatggaacaaatggcc

SEQ ID NO: 42 - DNA, 69 bp, synthetic construct
  misc_feature 1..69: note = Synthetic Construct
  source 1..69: mol_type = other DNA
ctggaggcttgctgaaggctgtatgctgacacaaggcctgttactagcactcacatggaacaaatggcc

SEQ ID NO: 43 - DNA, 145 bp, synthetic construct
  misc_feature 1..145: note = Synthetic Construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 44 - DNA, 1138 bp, synthetic construct; identical to SEQ ID NO: 18
  misc_feature 1..1138: note = Synthetic Construct
  source 1..1138: mol_type = other DNA

SEQ ID NO: 45 - DNA, 311 bp, Oryctolagus cuniculus
  source 1..311: mol_type = other DNA
cctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattc

SEQ ID NO: 46 - DNA, 34 bp, synthetic construct
  misc_feature 1..34: note = Synthetic Construct
  source 1..34: mol_type = other DNA
cggatccctggaggcttgctgaaggctgtatgct

SEQ ID NO: 47 - DNA, 283 bp, Oryctolagus cuniculus
  source 1..283: mol_type = other DNA
caatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctg

SEQ ID NO: 48 - DNA, 398 bp, synthetic construct; identical to SEQ ID NO: 19
  misc_feature 1..398: note = Synthetic Construct
  source 1..398: mol_type = other DNA

SEQ ID NO: 49 - DNA, 145 bp, synthetic construct
  misc_feature 1..145: note = Synthetic Construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 50 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSIFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 51 - DNA, 2217 bp, synthetic construct
  source 1..2217: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcatctttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 52 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFARTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 53 - DNA, 2217 bp, synthetic construct
  source 1..2217: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgccaggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa